SEQUENCE LISTING (8 sequences)

SEQ ID NO: 1 - protein, 902 aa, synthetic construct
  REGION 1..902: note = L274GMmMetRS
MRLFVSEGSPGSLPVLAAAARARGRAELLISTVGPEECVVPFLTRPKVPVLQLDSGNYLFSASAICRYFFLLCGWEQDDLTNQWLEWEATELQPVLSAALHCLVVQGKKGEDILGPLRRVLTHIDHSLSRQNCPFLAGDTESLADIVLWGALYPLLQDPAYLPEELGALQSWFQTLSTQEPCQRAAETVLKQQGVLALRLYLQKQPQPQPPPPEGRTVSNELEEEELATLSEEDIVTAVAAWEKGLESLPPLKLQQHPVLPVPGERNVLITSAGPYVNNVPHLGNIIGCVLSADVFARYCRLRQWNTLYLCGTDEYGTATETKAMEEGLTPREICDKYHAIHADIYRWFGISFDTFGRTTTPQQTKITQDIFQRLLTRGFVLRDTVEQLRCERCARFLADRFVEGVCPFCGYEEARGDQCDRCGKLINAIELKKPQCKICRSCPVVRSSQHLFLDLPKLEKRLEDWLGKTVPGSDWTPNARFIIRSWLRDGLKPRCITRDLKWGTPVPLEGFEDKVFYVWFDATIGYVSITANYTDQWEKWWKNPEQVDLYQFMAKDNVPFHGLVFPCSVLGAEDNYTLVKHIIATEYLNYEDGKFSKSRGIGVFGDMAKDTGIPADIWRFYLLYIRPEGQDSAFSWTDLLIKNNSELLNNLGNFINRAGMFVSKFFGGCVPEMALTPDDRRLVAHVSWELQHYHQLLEKVRIRDALRSILTISRHGNQYIQVNEPWKRIKGGEMDRQRAGTVTGMAVNMAALLSVMLQPYMPTVSSTIQTQLQLPPAACRILATSFICTLPAGHRIGTVSPLFQKLENDQIENLRQRFGGGQAKGSPKPAAVEAVTAAGSQHIQTLTDEVTKQGNVVRELKAQKADKNQVAAEVAKLLDLKKQLALAEGKPIETPKGKKKK

SEQ ID NO: 2 - protein, 508 aa, synthetic construct
  REGION 1..508: note = T413GMmPheRS
MADNPVLELLLRRLEVADGGLDSAELATQLGVEHQAVVGAVKSLQALGEVIEAELRSTKCWELTTEGEEIAREGSHEARVFRSIPLEGLVQSELMHLPSGKVGFSKAMSNKWIRVDKSAADGPRVFRVVDSIEDEVQKRLQLVQAGQAEKLAEKERNELRKRKLLTEVILKTYWVSKGKAFSTSVSKQEAELSPEMISSGSWRDRPFKPYNFSARGVLPDSGHLHPLLKVRSQFRQIFLEMGFTEMPTDNFIESSFWNFDALFQPQQHPARDQHDTFFLRDPAEALQLPMGYVQRVKRTHSQGGYGSQGYKYTWKLEEARKNLLRTHTTAASARALYQLAQKKPFTPAKYFSIDRVFRNETLDATHLAEFHQIEGVIADHGLTLGHLMGVLREFFTKLGITQLRFKPAYNPYGEPSMEVFSYHQGLKKWVEVGNSGVFRPEMLLPMGLPENVSVIAWGLSLERPTMIKYGINNIRELVGHKVNLQMVYDSPVCRLDIEPRSSKTQEAA

SEQ ID NO: 3 - protein, 394 aa, synthetic construct
  REGION 1..394: note = Y43GScTyrRS
MSSAATVDPNEAFGLITKNLQEVLNPQIIKDVLEVQKRHLKLGWGTAPTGRPHCGYFVPMTKLADFLKAGCEVTVLLADLHAFLDNMKAPLEVVNYRAKYYELTIKAILRSINVPIEKLKFVVGSSYQLTPDYTMDIFRLSNIVSQNDAKRAGADVVKQVANPLLSGLIYPLMQALDEQFLDVDCQFGGVDQRKIFVLAEENLPSLGYKKRAHLMNPMVPGLAQGGKMSASDPNSKIDLLEEPKQVKKKINSAFCSPGNVEENGLLSFVQYVIAPIQELKFGTNHFEFFIDRPEKFGGPITYKSFEEMKLAFKEEKLSPPDLKIGVADAINELLEPIRQEFADNKEFQEASEKGYPVATPQKSKKAKKPKNKGTKYPGATKTNEIATKLEETKL

SEQ ID NO: 4 - DNA, 97 bp, synthetic construct
  misc_feature 1..97: note = AREG KD1 shRNA coding sequence
  source 1..97: mol_type = other DNA
tgctgttgacagtgagcgaacagctgttgctgttattacatagtgaagccacagatgtatgtaataacagcaacagctgtgtgcctactgcctcgga

SEQ ID NO: 5 - DNA, 97 bp, synthetic construct
  misc_feature 1..97: note = AREG KD2 shRNA coding sequence
  source 1..97: mol_type = other DNA
tgctgttgacagtgagcgaatgcaaatgtcagcaagaatatagtgaagccacagatgtatattcttgctgacatttgcatgtgcctactgcctcgga

SEQ ID NO: 6 - DNA, 759 bp, Homo sapiens
  source 1..759: mol_type = other DNA
atgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataa

SEQ ID NO: 7 - DNA, 249 bp, synthetic construct
  misc_feature 1..249: note = ORF stuffer control
  source 1..249: mol_type = other DNA
gtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgagtggcgctttgcctggtttccggcaccagaagcggtgccggaaagctggctggagtgcgatcttcctgaggccgatactgtcgtcgtcccctcaaactggcag

SEQ ID NO: 8 - DNA, 2712 bp, synthetic construct
  misc_feature 1..2712: note = L274GMmMetRS coding sequence
  source 1..2712: mol_type = other DNA
atgagactgttcgtgagcgagggttccccggggagcctgcccgtgctggctgcggccgcgagggcccggggtcgggcggagctgctcatcagcaccgtaggccccgaagagtgtgtggtaccattccttacccggcctaaggtccctgtcttgcagctggatagtggcaactacctcttctctgctagtgcaatctgccgatatttttttctgttatgtggctgggaacaagatgatctcaccaaccagtggctggaatgggaggcaacagaactgcagccagttctgtctgctgccctacactgtctagtggttcaaggcaagaaaggggaagatatacttggcccacttcggagagtcctgactcacattgatcacagcttgagtcgtcaaaactgtcctttcctggctggggacacagaatctctagctgacattgttttgtggggagcactgtatcctttactgcaagacccagcttacctccctgaggagttgggtgccctgcaaagttggttccagacactgagtacccaggaaccgtgtcagcgagctgcagagacggtgctaaaacagcagggtgtcctggcacttcgtctgtacctccagaaacagccacagcctcagcccccgcctcctgaggggagaactgtcagcaacgagctggaggaagaggaactggctaccttgtctgaggaggacatcgttacagctgttgccgcgtgggagaagggtctggaaagcctgcctccgctaaagctccagcagcatccagtgttgcctgtgcctggagagaggaatgttctcatcaccagtgccggaccctatgtcaacaatgtcccccaccttggaaacatcattggctgtgtgctcagtgctgatgtctttgcaaggtattgtcgccttcgccagtggaataccctctatctgtgtggtacagatgagtatggtactgcgacagagaccaaggccatggaggagggcctaaccccacgggaaatctgtgacaagtaccatgccatccatgctgacatctaccgctggttcggcatatcgttcgatactttcgggcgcactaccactcctcagcagaccaaaatcacccaggacatcttccagaggttgctgacccgggggtttgtgctgcgagatactgtggagcagcttcggtgtgagcggtgtgcacgtttcctggctgaccgctttgtggagggtgtgtgtcccttctgtggctatgaagaggcccgaggtgaccagtgtgacaggtgtggcaagctcatcaatgccattgagctcaagaaaccacagtgcaaaatctgccgctcctgccctgtggtgaggtcctcacagcacctgtttctagacttgcctaagttggaaaagcgtctggaggactggttggggaagacagtgcctggcagtgactggacacccaatgccaggttcattatacgttcctggcttcgagatggcctcaagccacgatgcatcaccagagacctcaaatggggaacgcctgtgcccttggaaggttttgaggacaaggtattttacgtctggtttgatgctactattggctacgtgtccatcacagccaactacacagaccaatgggagaaatggtggaagaacccagaacaagtggacctttaccagttcatggccaaagacaatgttcccttccatggcttggtctttccgtgttcagtcctaggagctgaggacaactacaccctggtcaagcacatcattgctacagagtacctgaactatgaggatgggaaattctctaagagccggggcataggagtgtttggagacatggccaaggatacaggaatccctgctgacatctggcgattctatctgctatacattcggcctgagggccaggacagtgccttctcctggacagacttgttgattaaaaacaattctgagctgctcaacaacctgggcaacttcatcaacagagctggcatgtttgtttctaagttttttggcggttgtgtgcctgagatggcgctaacccctgatgacagacgcctggtggcccatgtctcttgggaactccagcactatcaccagctgttggaaaaggttcggatccgggatgccttgcgcagtatcctcaccatatctcgccatggcaaccaatacattcaagtgaatgagccctggaaacggattaaaggtggtgagatggacaggcagcgggcaggcacagtgacaggcatggcagtgaacatggctgccttgctgtctgtcatgctgcagccatacatgcccacagtcagctctaccatccagacccagctgcagctcccacctgcagcctgccgcatccttgccacaagcttcatttgtaccttgccagcaggccaccgaattggcacagtcagtcctttgttccaaaaactggaaaatgaccagattgaaaatttgaggcagcgctttggagggggtcaggctaaaggctcccccaagccagcagctgtggaggcagttacagcagcaggctcgcagcacatacaaacgctgacggatgaggtgaccaagcagggcaacgtcgtccgggaactgaaagcacagaaggcagacaagaaccaggttgctgcagaggtggctaaactcttggatctaaagaaacagttggctttggctgaggggaaacccattgaaactcctaaaggcaagaagaaaaagtgataa

(19) United States
(12) Patent Application Publication
(10) Pub. No.: US 2026/0200971 A1
(43) Pub. Date: Jul. 16, 2026

(54) METHODS FOR ISOLATING, DETECTING, AND QUANTIFYING INTERCELLULAR PROTEINS

(71) Applicant: Fred Hutchinson Cancer Center, Seattle, WA (US)

(72) Inventors: Kevin CHEUNG, Seattle, WA (US); Brad KRAJINA, Seattle, WA (US)

(73) Assignee: Fred Hutchinson Cancer Center, Seattle, WA (US)

(21) Appl. No.: 19/115,217

(22) PCT Filed: Sep. 26, 2023
(86) PCT No.: PCT/US2023/075159

§ 371 Date: Mar. 25, 2025

Related U.S. Application Data
(60) Provisional application No. 63/410,491, filed on Sep. 27, 2022.

(51) Int. Cl. C07K 1/13; C07K 1/14; C12P 21/00

(52) U.S. Cl. CPC C07K 1/13; C07K 1/14; C12P 21/00

(57) ABSTRACT

Disclosed are methods for isolating full-length intercellular proteins from a complex, multicellular environment. The disclosed methods include growing cells in the complex environment in the presence of an amino acid or alkanoic acid analog comprising a first chemical group. The first chemical group (e.g., an azide) is reactive in a bioorthogonal chemical reaction with a second chemical group (e.g., a strained alkyne) present in a cell-membrane-impermeable probe further containing a hydrophilic linker and a biotin analog moiety (e.g., desthiobiotin). The amino acid or alkanoic acid analog is incorporated into newly synthesized pericellular proteins, which are then labeled with the probe using the bioorthogonal reaction and isolated using an immobilized biotin-binding protein capture system. Captured proteins can then be retrieved in their full-length form using a mild elution process for further downstream analysis, including, e.g., protein detection and quantitation. The disclosed methods can be performed on cells grown in vitro, ex vivo, or in vivo.

### CROSS-REFERENCE TO RELATED APPLICATION

This application claims the benefit of U.S. Provisional Patent Application No. 63/410,491, filed Sep. 27, 2022, the disclosure of which is hereby incorporated by reference in its entirety.

### STATEMENT OF GOVERNMENT LICENSE RIGHTS

This invention was made with government support under W81XWH-18-1-0098 awarded by the Medical Research and Development Command, and CA234488 awarded by the National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P80WO_Seq_Listing_ST26.xml. The XML file is 16,207 bytes, was created on Sep. 18, 2023, and is being submitted electronically via Patent Center with the filing of the specification.

### BACKGROUND

Multicellular ecosystems communicate through the exchange of extracellular signaling molecules. For example, in tumors such as, e.g., breast cancer, such multicellular communication can enable tumor cells to cooperate in a range of contexts, including tumor invasion and outgrowth of distal metastases. Recently, a new mechanism of multicellular communication was discovered in which intercellular spaces sealed by cell-cell junctions (also referred to herein as “nanolumena”) can serve as reservoirs for growth factors to accumulate and amplify intercellular signals, thereby supporting metastasis. See Wrenn et al., Cell 183:395-410, 2020. Profiling the proteome of these intercellular spaces may reveal new therapeutic targets.

Three-dimensional organoid models are versatile platforms for modeling multicellular behavior in vitro and ex vivo. However, organoid culture typically requires the use of poorly defined, animal-derived extracellular matrices, such as Matrigel®. These exogenous matrices contain thousands of proteins that conceal cell-secreted factors in conventional proteomics approaches. Consequently, it is difficult to distinguish cell-secreted factors, which exist in low abundance, from the matrices' complex exogenous background.

Existing methods that have been conventionally used for pericellular proteomics suffer key disadvantages. For example, NHS-ester based cell-surface labeling methods react with exogenous proteins and, in 3D organotypic culture, NHS-ester-based technology is unable to penetrate intercellular spaces due to excessive reactivity with extracellular matrix (ECM)-based culture components on the exterior. In addition, subcellular fractionation techniques using detergents or ultracentrifugation do not allow for capture and detection of both membrane-bound and soluble proteins that are present in intercellular spaces. Further, proximity ligation-based biotinylation technologies (e.g., BioID, TurboID) require on-bead digestion of proteins for downstream analysis and do not allow for efficient elution of full-length proteins, thereby rendering such methods incompatible with certain downstream analytic techniques including, for example, a wide range of immunoaffinity based assays. Still further, protein labeling methods that rely on tagging of sugars present in glycoproteins (which are typically secreted) do not provide for unbiased labeling of all proteins present in intercellular spaces.

There is a need for proteome isolation and profiling methods that provide for capture of proteins in intercellular spaces (including, e.g., unbiased capture of such intercellular proteins) without capture of exogenous culture components or intracellular proteins, as well as for efficient isolation of such intercellular proteins in their intact, full-length form for downstream analysis. There is also a need for methods that provide for such capture of full-length intercellular proteins from tissues in vivo.

### SUMMARY

In some aspects, the present disclosure provides a method for isolating proteins that are pericellularly localized in a multicellular system. The method generally includes the following steps: (a) culturing a plurality of cells in a complex cell culture system, wherein the plurality of cells are cultured in the presence of an amino acid analog functionalized with a first chemical group that is reactive in a bioorthogonal chemical reaction, and wherein the amino acid analog is incorporated into newly synthesized proteins; (b) contacting the culture system with a cell-membrane-impermeable probe having the formula A-L-D, wherein A is a molecule comprising a second chemical group that is reactive in a bioorthogonal chemical reaction, wherein the first chemical group is reactive with second chemical group, L is a hydrophilic linker, and D is a molecule comprising a biotin analog moiety capable of specifically binding to streptavidin with a dissociation constant greater than about 1 pM and an off-rate kinetic constant greater than about 1×10⁻³ l/s, and wherein newly synthesized proteins that are pericellularly localized are labeled with the probe by a bioorthogonal chemical reaction between the first and second chemical groups; (c) lysing the cultured cells to generate a lysate containing the labeled proteins and other cellular and extracellular components; (d) contacting the lysate with a capture reagent comprising a biotin-binding protein immobilized on a solid support, wherein the biotin-binding protein binds to the biotin analog moiety on the labeled proteins to yield a [labeled protein]:probe complex; and (e) separating the solid support from the lysate to separate the labeled proteins from the other cellular and extracellular components, thereby isolating the proteins that are pericellularly localized in the multicellular system. In some embodiments, the proteins that are pericellularly localized are nanolumenal proteins.

In other aspects, the present disclosure provides a method for isolating proteins that are pericellularly localized in an animal in vivo. The method generally includes the following steps: (a) implanting into an animal a plurality of cells, wherein the plurality of cells are genetically engineered to express a mutant t-RNA synthetase that selectively incorporates a mutant-specific amino acid analog; (b) administering the mutant-specific amino acid analog to the animal, wherein the mutant-specific amino acid analog is functionalized with a first chemical group that is reactive in a bioorthogonal chemical reaction, and whereby the mutant-specific amino acid analog is selectively incorporated into proteins synthesized by the implanted, genetically engineered cells but not into proteins synthesized by the other cells within the animal; (c) administering a cell-membrane-impermeable probe to the animal, wherein the probe has the formula A-L-D, wherein A is a molecule comprising a second chemical group that is reactive in a bioorthogonal chemical reaction, wherein the first chemical group is reactive with second chemical group, L is a hydrophilic linker, and D is a molecule comprising a biotin analog moiety capable of specifically binding to streptavidin with a dissociation constant greater than about 1 pM and an off-rate kinetic constant greater than about 1×10⁻³ l/s, and wherein proteins that are synthesized by the implanted cells and pericellularly localized are labeled with the probe by a bioorthogonal chemical reaction between the first and second chemical groups; (c) harvesting a tissue comprising the implanted cells from the animal; (d) lysing the harvested tissue to generate a lysate containing the labeled proteins and other cellular and extracellular components; (e) contacting the lysate with a capture reagent comprising a biotin-binding protein immobilized on a solid support, wherein the biotin-binding protein binds to the biotin analog moiety on the labeled proteins to yield a [labeled protein]:probe complex; and (f) separating the solid support from the lysate to separate the labeled proteins from the other cellular and extracellular components, thereby isolating the proteins that are pericellularly localized in the animal. In some embodiments, the proteins that are pericellularly localized are nanolumenal proteins.

In still other aspects, the present disclosure provides a method for isolating lipidated proteins that are pericellularly localized in a multicellular system. The method generally includes the following steps: (a) culturing a plurality of cells in a complex cell culture system, wherein the plurality of cells are cultured in the presence of an alkanoic acid analog functionalized with a first chemical group that is reactive in a bioorthogonal chemical reaction, and wherein the alkanoic acid analog is incorporated into newly synthesized lipidated proteins; (b) contacting the culture system with a cell-membrane-impermeable probe having the formula A-L-D, wherein A is a molecule comprising a second chemical group that is reactive in a bioorthogonal chemical reaction, wherein the first chemical group is reactive with second chemical group, L is a hydrophilic linker, and D is a molecule comprising a biotin analog moiety capable of specifically binding to streptavidin with a dissociation constant greater than about 1 pM and an off-rate kinetic constant greater than about 1×10⁻³ l/s, and wherein newly synthesized lipidated proteins that are pericellularly localized are labeled with the probe by a bioorthogonal chemical reaction between the first and second chemical groups; (c) lysing the cultured cells to generate a lysate containing the labeled proteins and other cellular and extracellular components; (d) contacting the lysate with a capture reagent comprising a biotin-binding protein immobilized on a solid support, wherein the biotin-binding protein binds to the biotin analog moiety on the labeled proteins to yield a [labeled protein]:probe complex; and (e) separating the solid support from the lysate to separate the labeled proteins from the other cellular and extracellular components, thereby isolating the lipidated proteins that are pericellularly localized in the multicellular system. In some embodiments, the lipidated proteins that are pericellularly localized are nanolumenal proteins.

In some variations of a method as above, the biotin analog moiety capable of specifically binding to streptavidin with a dissociation constant greater than about 1 pM and an off-rate kinetic constant greater than about 1×10⁻³ l/s is selected from a desthiobiotin moiety, an N3′-ethyl biotin moiety, and a 2-iminobiotin moiety. In some such variations, the biotin analog moiety has the formula D1, D2, or D3

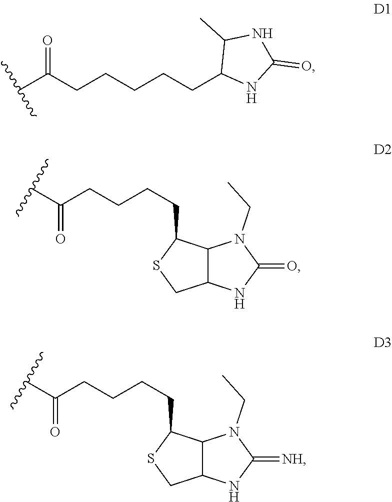 [SMILES: *C(=O)CCCCCC1NC(=O)NC1C ; *C(=O)CCCC[C@@H]1SCC2NC(=O)N(CC)C21 ; *C(=O)CCCC[C@@H]1SCC2NC(=N)N(CC)C21]

wherein [symbol] designates a point of attachment.

Suitable biotin-binding proteins for capture of the labeled proteins in a method as above include streptavidin and avidin. In certain non-mutually exclusive embodiments, the solid support is a magnetic bead solid support.

In some embodiments of a method as above, the method further includes eluting the labeled proteins from the immobilized biotin-binding protein, wherein the labeled proteins remain intact as full-length proteins following elution. In some such embodiments, eluting the labeled proteins from the immobilized biotin-binding protein comprises competitive elution with free biotin.

In certain embodiments, a method as above further includes analyzing one or more of the isolated proteins. Particularly suitable analytical techniques include LC/MS, MALDI-TOF, HPLC, ELISA, Western blot analysis, antibody array analysis, or Proximity Extension Assay (PEA). In some variations, analyzing the one or more isolated proteins comprises identifying the one or more isolated proteins. In some such variations, identifying the one or more isolated proteins comprises an immunoaffinity detection assay. Suitable immunoaffinity detection assays include ELISA, Western blot analysis, antibody array analysis, and Proximity Extension Assay (PEA). In other, non-mutually exclusive embodiments, analyzing the one or more isolated proteins includes quantifying the one or more isolated proteins.

In some embodiments of a method as above, the first chemical group that is reactive in a bioorthogonal reaction is an azide. In some such embodiments, the second chemical group that is reactive in a bioorthogonal chemical reaction is a strained alkyne. Particularly suitable molecules comprising a strained alkyne include cyclooctynes such as, e.g., dibenzocyclooctyne (DIBO/DBCO), OCT cyclooctyne, monofluorinated cyclooctyne (MOFO), difluorocyclooctyne (DIFO), dimethoxyazacyclooctyne (DIMAC), dibenzoazacyclooctyne (DIBAC), biarylazacyclooctynone (BARAC), bicyclononyne (BCN), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), and aryl-less octyne (ALO).

In certain variations of a method as above, the hydrophilic linker is non-charged. In alternative variations, the hydrophilic linker is negatively charged.

In some embodiments, the hydrophilic linker is a hydrophilic polymer comprising at least four monomeric residues. In some such embodiments, the hydrophilic linker comprises a plurality of non-charged monomeric residues such as, for example, polyethylene glycol (PEG) monomeric residues, polyvinyl alcohol (PVA) monomeric residues, or polyvinylpyrrolidone (PVP) monomeric residues. In certain variations wherein the non-charged residues are PEG monomeric residues, the hydrophilic linker contains from four to 16 PEG (e.g., four) monomeric residues. In certain embodiments of a hydrophilic linker comprising form four toe 16 PEG monomeric residues, the hydrophilic linker has the formula L1

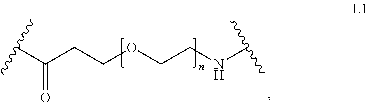

wherein n is an integer from four to 16, and [symbol] designates a point of attachment.

In certain variations of a hydrophilic linker comprising a plurality of non-charged monomeric residues, the hydrophilic linker further comprises at least one negatively charged residue. Suitable negatively charged residues include residues comprising an anionic group selected from a sulfonate, a phosphate, a phosphonate, and a carboxylic acid. In some variations wherein the at least one negatively charged residue comprises a sulfonate, the hydrophilic linker has the formula L4

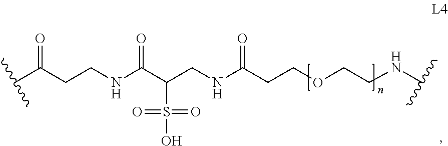

wherein n is an integer from 4 to 16, and [symbol] designates a point of attachment.

In certain embodiments wherein the hydrophilic linker is a hydrophilic polymer comprising at least four monomeric residues, the linker comprises a plurality of negatively charged monomeric residues. In some such embodiments, each of the plurality of negatively charged residues comprises an anionic group selected from the group consisting of a sulfonate, a phosphate, a phosphonate, and a carboxylic acid.

In certain embodiments wherein the hydrophilic linker is a negatively charged linker, the linker comprises at least one anionic group selected from the group consisting of a sulfonate, a phosphate, a phosphonate, and a carboxylic acid. In some such embodiments wherein the at least one anionic group is a sulfonate, the hydrophilic linker has the formula L2 or L3

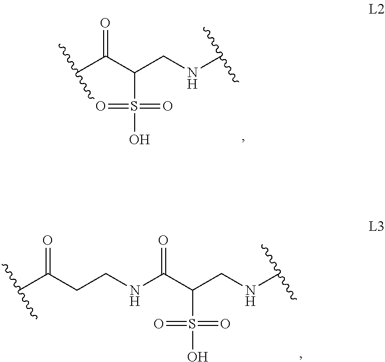 [SMILES: *NCC(C(*)=O)S(=O)(=O)O ; *NCC(C(=O)NCCC(*)=O)S(=O)(=O)O]

wherein [symbol] designates a point of attachment.

In some specific variations of a method as above, the cell-membrane-impermeable probe has the formula P1, P2, or P3

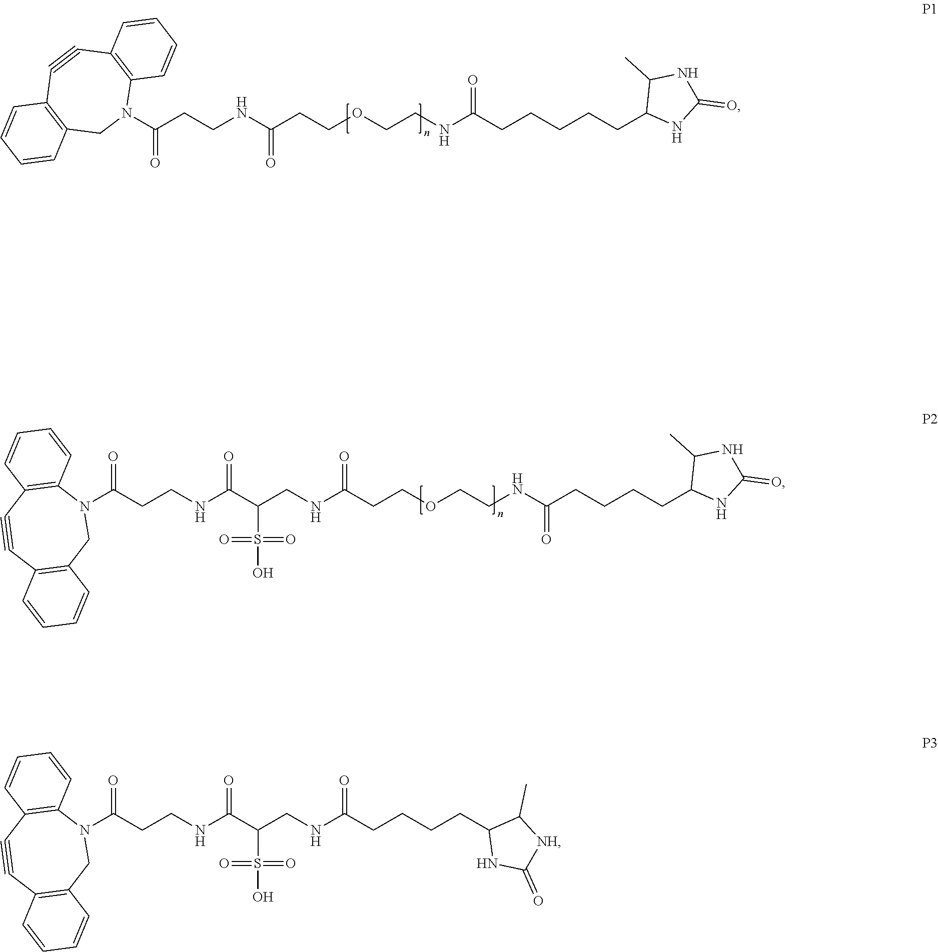

wherein n is an integer from 4 to 16.

In certain embodiments of a method above wherein the method is for isolating proteins that are pericellularly localized in a multicellular system or lipidated proteins that are pericellularly localized in a multicellular system, the complex cell culture system is a three-dimensional (3D) cell culture system. Particularly suitable 3D cell culture systems include 3D hydrogel systems such as, for example, a hydrogel system comprising extracellular matrix (ECM)-derived hydrogel (e.g., an ECM-derived hydrogel derived from Engelbreth-Holm-Swarm (EHS) mouse sarcoma).

In some embodiments of a method above wherein the method is for isolating proteins that are pericellularly localized in a multicellular system or lipidated proteins that are pericellularly localized in a multicellular system, the plurality of cells are from a cell line. In alternative variations, the plurality of cells are from a primary tissue. Suitable cells (either from a cell line or from a primary tissue) include cells selected from fibroblasts, epithelial cells, endothelial cells, neuronal cells, glial cells, lymphocytes, macrophages, dendritic cells, hepatocytes, and stem cells. Particularly suitable cells (either from a cell line or from a primary tissue) also include tumor cells. In some variations comprising tumor cells, the tumor cells are solid tumor cells, which may be cells from a cancer selected from breast cancer (e.g., estrogen receptor positive (ER+) breast cancer or triple negative breast cancer), small-cell lung cancer, non-small cell lung cancer, hepatocellular carcinoma, melanoma, renal cell carcinoma, basal cell carcinoma, cutaneous squamous cell carcinoma, esophageal cancer, pancreatic cancer, glioblastoma, cervical cancer, ovarian cancer, urothelial carcinoma, gastric carcinoma, colon cancer, colorectal cancer, testicular cancer, and Merkel cell carcinoma. In other variations, the tumor cells are non-solid tumor cells. In particular embodiments wherein the cells are ER+ breast cancer cells, the cells are cultured in the presence of estradiol. In some non-mutually exclusive embodiments, the tumor cells are cultured in the presence of an anti-cancer drug. For example, in some embodiments wherein the tumor cells are breast cancer cells, the cells are cultured in the presence of a hormonal therapeutic drug such as, e.g., tamoxifen or fulvestrant.

In some embodiments of a method as above wherein the method is for isolating proteins that are pericellularly localized in a multicellular system or lipidated proteins that are pericellularly localized in a multicellular system, the plurality of cells comprises at least two different cell types. In some such embodiments, the at least two cell types comprise a tumor cell and an immune cell.

In some embodiments of a method as above wherein the method is for isolating proteins that are pericellularly localized in a multicellular system, the first chemical group that is reactive in a bioorthogonal chemical reaction is an azide and the amino acid analog is azidohomoalanine. In other variations wherein the plurality of cells comprises at least two different cell types, the amino acid analog is a mutant-specific amino acid analog that is selectively incorporated by a mutant t-RNA synthetase one of the at least two different cell types is a genetically engineered cell expressing the mutant t-RNA synthetase, whereby the mutant-specific amino acid analog is selectively incorporated into proteins synthesized by the genetically engineered cell but not into proteins synthesized by the other cell type(s) within the culture system. In some such embodiments, the mutant t-RNA synthetase is L274GMmMetRS and the amino acid analog is azidonorleucine, the mutant t-RNA synthetase is T413GMmPheRS and the amino acid analog is p-azido-L-phenylalanine, or the mutant t-RNA synthetase is Y43GScTyrRS and the amino acid analog is 3-azido-L-tyrosine.

In certain embodiments of a method as above wherein the method is for isolating proteins that are pericellularly localized in an animal in vivo, the implanted cells are tumor cells. In some such variations, the tumor cells are solid tumor cells, which may be cells from a cancer selected from breast cancer (e.g., estrogen receptor positive (ER+) breast cancer or triple negative breast cancer), small-cell lung cancer, non-small cell lung cancer, hepatocellular carcinoma, melanoma, renal cell carcinoma, basal cell carcinoma, cutaneous squamous cell carcinoma, esophageal cancer, pancreatic cancer, glioblastoma, cervical cancer, ovarian cancer, urothelial carcinoma, gastric carcinoma, colon cancer, colorectal cancer, testicular cancer, and Merkel cell carcinoma. In other variations, the tumor cells are non-solid tumor cells. In particular embodiments wherein the cells are ER+ breast cancer cells, animal is treated with estradiol before step (c). In some non-mutually exclusive embodiments, the animal is treated with an anti-cancer drug before step (c). For example, in some embodiments wherein the tumor cells are breast cancer cells, the animal is treated with a hormonal therapeutic drug such as, e.g., tamoxifen or fulvestrant.

In some variations of a method as above wherein the method is for isolating proteins that are pericellularly localized in an animal in vivo, the mutant t-RNA synthetase is L274GMmMetRS and the amino acid analog is azidonorleucine, the mutant t-RNA synthetase is T413GMmPheRS and the amino acid analog is p-azido-L-phenylalanine, or the mutant t-RNA synthetase is Y43GScTyrRS and the amino acid analog is 3-azido-L-tyrosine.

In some embodiments of a method as above wherein the method is for isolating lipidated proteins that are pericellularly localized in a multicellular system, the alkanoic acid analog is a myristic acid analog or a palmitic acid analog. In some such embodiments wherein the first chemical group is an azide, the alkanoic acid analog is 12-azidododecanoic acid or 15-azidopentadecanoic acid.

In some related aspects of the aforedescribed method for isolating proteins that are pericellularly localized in a multicellular system, the present disclosure provides a method that generally includes the following steps: (a) culturing a plurality of cells in a complex cell culture system, wherein the plurality of cells are cultured in the presence of an amino acid analog functionalized with a first chemical group that is reactive in a bioorthogonal chemical reaction, and wherein the amino acid analog is incorporated into newly synthesized proteins; (b) contacting the culture system with a cell-membrane-impermeable probe having the formula A-L-D, wherein A is a molecule comprising a second chemical group that is reactive in a bioorthogonal chemical reaction, wherein the first chemical group is reactive with second chemical group, L is a hydrophilic linker, and D is a means for specifically binding to streptavidin with a dissociation constant greater than about 1 pM and an off-rate kinetic constant greater than about 1×10⁻³ l/s, and wherein newly synthesized proteins that are pericellularly localized are labeled with the probe by a bioorthogonal chemical reaction between the first and second chemical groups; (c) lysing the cultured cells to generate a lysate containing the labeled proteins and other cellular and extracellular components; (d) contacting the lysate with a capture reagent comprising streptavidin immobilized on a solid support, wherein the streptavidin binds to the means for specifically binding to streptavidin on the labeled proteins to yield a [labeled protein]:probe complex; and (e) separating the solid support from the lysate to separate the labeled proteins from the other cellular and extracellular components, thereby isolating the proteins that are pericellularly localized in the multicellular system. In some embodiments, the method further includes eluting the labeled proteins from the immobilized streptavidin, wherein the labeled proteins remain intact as full-length proteins following elution. In some such embodiments, eluting the labeled proteins from the immobilized streptavidin comprises competitive elution with free biotin.

In some related aspects of the aforedescribed method for isolating proteins that are pericellularly localized in an animal in vivo, the present disclosure provides a method that generally includes the following steps: (a) implanting into an animal a plurality of cells, wherein the plurality of cells are genetically engineered to express a mutant t-RNA synthetase that selectively incorporates a mutant-specific amino acid analog; (b) administering the mutant-specific amino acid analog to the animal, wherein the mutant-specific amino acid analog is functionalized with a first chemical group that is reactive in a bioorthogonal chemical reaction, and whereby the mutant-specific amino acid analog is selectively incorporated into proteins synthesized by the implanted, genetically engineered cells but not into proteins synthesized by the other cells within the animal; (c) administering a cell-membrane-impermeable probe to the animal, wherein the probe has the formula A-L-D, wherein A is a molecule comprising a second chemical group that is reactive in a bioorthogonal chemical reaction, wherein the first chemical group is reactive with second chemical group, L is a hydrophilic linker, and D is a means for specifically binding to streptavidin with a dissociation constant greater than about 1 pM and an off-rate kinetic constant greater than about 1×10⁻³ l/s, and wherein proteins that are synthesized by the implanted cells and pericellularly localized are labeled with the probe by a bioorthogonal chemical reaction between the first and second chemical groups; (c) harvesting a tissue comprising the implanted cells from the animal; (d) lysing the harvested tissue to generate a lysate containing the labeled proteins and other cellular and extracellular components; (e) contacting the lysate with a capture reagent comprising streptavidin immobilized on a solid support, wherein the streptavidin binds to the means for specifically binding to streptavidin on the labeled proteins to yield a [labeled protein]:probe complex; and (f) separating the solid support from the lysate to separate the labeled proteins from the other cellular and extracellular components, thereby isolating the proteins that are pericellularly localized in the animal. In some embodiments, the method further includes eluting the labeled proteins from the immobilized streptavidin, wherein the labeled proteins remain intact as full-length proteins following elution. In some such embodiments, eluting the labeled proteins from the immobilized streptavidin comprises competitive elution with free biotin.

In some related aspects of the aforedescribed method for isolating lipidated proteins that are pericellularly localized in a multicellular system, the present disclosure provides a method that generally includes the following steps: (a) culturing a plurality of cells in a complex cell culture system, wherein the plurality of cells are cultured in the presence of an alkanoic acid analog functionalized with a first chemical group that is reactive in a bioorthogonal chemical reaction, and wherein the alkanoic acid analog is incorporated into newly synthesized lipidated proteins; (b) contacting the culture system with a cell-membrane-impermeable probe having the formula A-L-D, wherein A is a molecule comprising a second chemical group that is reactive in a bioorthogonal chemical reaction, wherein the first chemical group is reactive with second chemical group, L is a hydrophilic linker, and D is a means for specifically binding to streptavidin with a dissociation constant greater than about 1 pM and an off-rate kinetic constant greater than about 1×10⁻³ l/s, and wherein newly synthesized lipidated proteins that are pericellularly localized are labeled with the probe by a bioorthogonal chemical reaction between the first and second chemical groups; (c) lysing the cultured cells to generate a lysate containing the labeled proteins and other cellular and extracellular components; (d) contacting the lysate with a capture reagent comprising streptavidin immobilized on a solid support, wherein the streptavidin binds to the means for specifically binding to streptavidin on the labeled proteins to yield a [labeled protein]:probe complex; and (e) separating the solid support from the lysate to separate the labeled proteins from the other cellular and extracellular components, thereby isolating the lipidated proteins that are pericellularly localized in the multicellular system. In some embodiments, the method further includes eluting the labeled proteins from the immobilized streptavidin, wherein the labeled proteins remain intact as full-length proteins following elution. In some such embodiments, eluting the labeled proteins from the immobilized streptavidin comprises competitive elution with free biotin.

These and other aspects and embodiments of the invention will become evident upon reference to the following detailed description of the invention.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

As used herein, the term “complex cell culture system” refers to either (i) a three-dimensional (3D) culture system or (ii) a two-dimensional (2D) culture system that involves the use of complex exogenous protein supplements (e.g., fetal bovine serum).

As used herein, the term “three-dimensional (3D) culture system” refers to a culture system that allows a plurality of cultured cells to form three-dimensional multicellular structures such as, e.g., spheroids. Examples of 3D culture systems include cultures in a concentrated medium or in a gel-like substance (e.g., a hydrogel), cultures on a scaffold, (e.g., an extracellular matrix (ECM) scaffold or a fibrous scaffold derived from a natural or synthetic polymer), and 3D suspension culture systems. Examples of hydrogel systems for 3D cell culture include, for example, soft agar and ECM-based hydrogels such as, e.g., Matrigel®, Cultrex™, and Geltrex™.

A “protein” is a macromolecule comprising one or more polypeptide chains. A “polypeptide” is a polymer of amino acid residues joined by peptide bonds. In the context of the present disclosure, proteins are produced naturally within cells in vitro, ex vivo, or in vivo. A protein may also comprise non-peptidic components, such as carbohydrate or lipid groups, which may be added by the cell in which the protein is produced.

“Pericellularly localized,” in reference to a cellular protein, means a protein that is localized to a space surrounding and between individual cells in a multicellular system. Proteins that are pericellularly localized are also referred to herein as “pericellular proteins” or “intercellular proteins.” In some variations, pericellular proteins are localized within “nanolumina,” i.e., intercellular compartments sealed by cell-cell junctions (and which may be lined with microvilli-like protrusions). Proteins localized within nanolumina are also referred to herein as “nanolumenal proteins.” Pericellular proteins may be cell-membrane-bound or secreted.

As used herein, the term “bioorthogonal” is used to refer to a chemical reaction that can occur in vitro, ex vivo, or in vivo in living systems without interfering with native biochemical processes. A bioorthogonal “click reaction” refers to a high-yielding and highly specific biorthogonal chemical reaction between substrates having a low activation energy barrier. In some embodiments, a bioorthogonal click reaction is 1,3-dipolar cycloaddition between an azide and an alkyne. Complementary pairs of bioorthogonal chemical groups are also referred to herein as a “first chemical group” and “second chemical group” that are reactive in a bioorthogonal chemical reaction.

The term “amino acid analog,” as used herein, means an unnatural amino acid that can serve as a surrogate for one of the twenty natural amino acids specified by codons of the genetic code in eurkaryotic cells, such that the amino acid analog, when present in cells, can be incorporated into a protein in place of the corresponding natural amino acid during translation.

The phrase “amino acid analog functionalized with a first chemical group” or “alkanoic acid analog functionalized with a first chemical group” means an amino acid or alkanoic acid analog comprising the first chemical group as a part of its molecular structure.

“Polyethylene glycol” and “PEG” are used interchangeably with “polyethylene oxide,” and are understood to mean an oligomer or polymer of —CH₂—CH₂—O— repeat units (which repeat units may also be referred to as “ethylene glycol units,” “ethylene oxide units,” or “PEG monomeric residues”). As is well-known in the art, nomenclature of PEG length can use the number of repeat units. For example, a PEG chain having four monomeric residues is also referred to as “PEG4.”

The term “lyse” or “lysing,” as used herein in reference to cells or a tissue, refers generally to a process whereby cell membranes are disrupted or destroyed in order to release cellular components, including cell membrane-bound proteins. Lysis may also help release other, non-membrane-bound pericellular proteins from intercellular compartments (e.g., from nanolumina) and/or break down extracellular components of a tissue or culture system. The process may be chemical, physical, enzymatic, or any suitable combination thereof. The resulting lysed material is referred to herein as a “lysate.”

The term “isolate” or “isolating,” in reference to pericellular proteins, means that the pericellular proteins are removed or separated from other components of a multicellular system. Other components of a multicellular system may include carbohydrates, lipids, nucleic acids, and other proteins, including intracellular proteins as well as extracellular proteins that are exogenous to the multicellular system being interrogated (e.g., exogenous extracellular matrix proteins that may be used in a 3D culture system). “Isolating” does not connote any degree of purification. Typically, at least 70%, at least 80%, or at least 95% of pericellular proteins are separated or removed from other components of a multicellular system.

The phrase “genetically engineered cell expressing a mutant t-RNA synthetase,” as used herein, means a cell comprising a transgene encoding and expressing the mutant t-RNA synthetase, which may be the result of either non-viral transfection or viral transduction of the transgene into a cell, leading to either transient or stable expression of the transgene.

To the extent used herein, the terms “first” and “second” preceding the name of an element (e.g., a chemical group) are used for identification purposes to distinguish between similar elements, and are not intended to necessarily imply order, nor are the terms “first” and “second” intended to preclude the inclusion of additional similar elements.

When a value is expressed as “about” X or “approximately” X, the stated value of X will be understood to be accurate to ±10%.

### DESCRIPTION OF THE DRAWINGS

FIG. 1 shows a schematic summary of certain embodiments of the disclosed method. (1) Multicellular clusters are cultivated in 3D culture containing extracellular matrix. (2) An azide amino acid analog (e.g., azidohomoalanine) is added to the media, allowing newly synthesized proteins to incorporate the azide tag. (3) A membrane impermeable click probe is then added to the culture (e.g., DBCO-PEG4-Desthiobiotin) which labels azide-modified proteins in intercellular spaces. (4) The cells are then lysed and contacted with magnetic beads conjugated to streptavidin to bind labeled intercellular proteins. (5) The full-length proteins are then eluted, at which point they are suitable for a range of proteomics analysis methods, including LC/MS, ELISA, western blotting, and the proximity extension assay (PEA).

FIG. 2 shows that membrane impermeable DBCO click probes—but not sulfo-NHS reagent—can label azidohomoalanine-modified intercellular proteins in 3D spheroid culture containing exogeneous extracellular matrix. Fluorescence confocal microscopy was used to evaluate each probe's ability to label intercellular spaces in HCC70 spheroids treated with azidohomoalanine (500 pM) and cultured in 2% Engelbreth-Holm-Swarm (EHS) basement membrane-rich matrix suspension culture. While the sulfo-NHS-biotin reagent (far left panels) was unable to penetrate beyond the EHS matrix and label intercellular spaces, the DBCO-containing bio-orthogonal click reagents (middle and far right panels) diffused into intercellular spaces of HCC70 cells cultured with azidohomoalanine and specifically labeled pericellular proteins. Nuclei were counterstained with DAPI, and the pericellular space was visualized by staining the cortical F-actin with phalloidin-568. Scale bar=50 μm.

FIGS. 3A-3D show that DBCO-PEG4-Desthiobiotin labels azidohomoalanine-modified proteins but not EHS matrix proteins. FIG. 3A shows fluorescent confocal micrographs of HCC70 spheroids cultured in 2% EHS matrix suspension culture, treated with or without azidohomoalanine, and subjected to labeling with DBCO-PEG4-Desthiobiotin, followed by desthiobiotin detection with fluorescent streptavidin labeling. Cells cultured in the presence of azidohomoalanine labeling showed substantially higher signal. Scale=25 μm. FIG. 3B shows representative micrographs of streptavidin dot blots of lysates from HCC70 cells, cultured as in FIG. 3A. The fluorescent intensity of DBCO-PEG4-Desthiobiotin labeled azidohomoalanine-modified proteins from lysate were significantly higher than controls. FIG. 3C shows streptavidin dot blots of lysates prepared from HCC70 cells cultured in fully embedded EHS matrix hydrogel culture and subjected to either azidohomoalanine/DBCO-PEG4-Desthiobiotin labeling or sulfo-NHS-Biotin. As a control, a cell-free EHS matrix hydrogel was subjected to the same labeling. In the cell-free hydrogel, the dot blots of the sulfo-NHS-Biotin reagent showed substantial off-target labeling that was distinct from the click DBCO-PEG4-Desthiobiotin. FIG. 3D shows fluorescent confocal micrographs of HCC70 cells cultured in fully embedded 3D EHS matrix culture and subjected to azidohomoalanine, DBCO-PEG4-Desthiobiotin, and streptavidin-alexafluor488 labeling. The cortical F-actin was counterstained with phalloidin-568 to visualize the location of the pericellular zone. Scale bar=25 μm.

FIGS. 4A-4D show that click-based Desthiobiotin labeling enables more efficient elution of full-length intercellular proteins than biotin labeling. FIG. 4A shows an elution experiment in which HCC70 spheroids in 2% EHS matrix suspension were treated either with DBCO-sulfo-biotin or DBCO-PEG4-Desthiobiotin, labeled proteins were captured in lysates with streptavidin beads, and elution was performed. The western blot shows signal intensity in the eluate for Claudin 7, a well-known intercellular protein. A quantification of the Claudin7 fluorescence intensity in the western blot is shown (mean of n=2 replicates, error bars are standard error of the mean). FIG. 4B shows western blot confirmation of enrichment of intercellular proteins (E-cadherin, Claudin 7) and depletion of cytoplasmic proteins (GAPDH, B-tubulin) in eluates from DBCO-PEG4-Desthiobiotin labeling, streptavidin enrichment, and mild elution with free biotin, in comparison to a crude lysate. FIG. 4C shows immunofluorescent confocal micrographs that validate the intercellular localization of E-cadherin and Claudin 7 and cytoplasmic localization of Beta-tubulin in HCC70 spheroids cultured in 2% EHS matrix suspension. The pericellular zone was visualized by counterstaining the cortical F-actin with phalloidin, and nuclei were counterstained with DAPI. Scale bar=25 μm.

FIGS. 5A-5C show that DBCO-PEG4-Desthiobiotin labeling and full-length protein recovery enables PEA-based discovery and quantification of intercellular signaling factors not found using LC/MS and on-bead digestion approaches. FIG. 5A shows a Venn diagram of proteins identified (FDR<0.01) by 1) LC/MS on crude lysates versus 2) intercellular protein enrichment by DBCO-PEG4-Desthiobiotin labeling, streptavidin capture and elution of full-length proteins, and proximity extension assay (PEA) quantification (Olink Explore 3072 protocol). Samples were HCC70 spheroids cultured in 2% EHS matrix suspension culture. FIG. 5B shows a Venn diagram of proteins identified (FDR<0.01) by either 1) LC/MS after labeling with DBCO-sulfo-biotin, performing immobilization on streptavidin magnetic beads, and performing tryptic on-bead digestion to release tryptic peptides versus 2) intercellular proteins enriched by DBCO-PEG4-Desthiobiotin labeling, streptavidin capture and elution of full-length proteins, and proximity extension assay (PEA) quantification (Olink Explore 3072 protocol). FIG. 5C shows an UpSet plot of proteins identified by each of the methods in FIGS. 5A and 5B that bear Uniprot KB annotations of “growth factor activity,” “cytokine activity,” or “hormone activity.”

FIGS. 6A-6B show that DBCO-PEG4-Desthiobiotin click labeling depleted contaminating EHS matrix proteins in 3D culture. FIG. 6A shows the LC/MS of crude lysates prepared from HCC70 cells cultured in 2% EHS matrix suspension culture. The normalized abundance (label free quantification) of the most abundant proteins is shown, with known EHS matrix proteins (Laminin B1, Laminin C1, Nidogen) indicated. FIG. 6B shows a comparison of the normalized abundances of EHS matrix proteins in crude HCC70 lysates (as in panel FIG. 6A) versus eluted, full-length proteins obtained from DBCO-PEG4-Desthiobiotin click labeling and streptavidin enrichment. Abundances were obtained from label-free quantification of LC/MS for both conditions. Error bars represent the standard error of the mean of biological replicates (n=2).

FIGS. 7A-7E show that DBCO-PEG4-Desthiobiotin click labeling with PEA-based quantification enables discovery and quantification of supraphysiological concentrations of intercellular growth factors (e.g., AREG) in ER+ breast cancer. FIG. 7A shows a schematic of the experimental design. FIG. 7B shows the absolute quantification of intercellular proteins in MC7:WS8 cells cultured in 2% EHS matrix, as determined by DBCO-PEG4-Desthiobiotin click labeling, streptavidin bead enrichment, full-length protein elution, and PEA quantification. The range of HER-2 abundances that has been observed in HER-2 positive breast cancer patients is indicated in the shaded region (see Onsum et al., Am. J. Pathol. 183:1446-1460, 2013). FIG. 7C shows a volcano plot of differential expression of intercellular proteins (as determined by click labeling and PEA) in MCF7:WS8 cells between cells treated with estradiol (1 nM) or vehicle control. FIG. 7D shows the relative intercellular expression of AREG (as determined by click labeling and PEA) in MCF7:WS8 cells subjected to the indicated treatments (E2=estradiol, 40HT=4-hydroxy tamoxifen, ICI=fulvestrant). FIG. 7E shows immunofluorescent confocal micrographs of MCF7:WS8 spheroids treated with or without estradiol (1 nM), confirming intercellular localization of AREG and its dependence on estradiol. Nuclei were counterstained with DAPI. Scale bar=25 μm.

FIGS. 8A-8B show that AREG, an intercellular growth factor identified using the disclosed method, promotes growth of ER+ breast cancer cells through intercellular communication. FIG. 8A shows immunofluorescent confocal micrographs of AREG expression in MCF7:WS8 cells that were subjected to lentiviral transductions with either a non-targeting shRNA or an AREG KD shRNA. The AREG KD shRNA generated a mosaic population of AREG high and low cells, which were sorted to produce an AREG low population. Nuclei were counterstained with DAPI. Scale bar=10 μm. FIG. 8B shows cell proliferation, as determined by a CyQuant genomic DNA quantification assay, in MCF7:WS8 cells representing the non-targeting, AREG mosaic, or AREG low populations for two different AREG shRNA hairpins. Values are represented mean+/−the standard error of the mean of n=8 replicate wells.

FIGS. 9A-9C show that the overexpression of AREG promotes resistance to endocrine therapy and PI3K inhibition. Dose response curves are shown for three different MCF7 sublines (MCF7:WS8, MCF7:PIK3CA-mutant, MCF7:PIK3CA-WT) in response to treatment with fulvestrant or alpelisib. Proliferation was determined by a CyQuant genomic DNA quantification. Individual points represent the mean of n=8 replicate wells. Curves represent the least-squares fit of the Hill equation. Error bands represent the 95% confidence interval of the fit, as determined using the gradient method.

FIGS. 10A-10C show that intercellular proteins are upregulated in response to endocrine therapy in ER+ breast cancer. FIG. 10A shows a schematic of the experimental design. FIG. 10B shows a volcano plot of differential expression of intercellular proteins (as determined by DBCO-PEG4-Desthibioitin click labeling and PEA quantification) in MCF7:WS8 3D cultures treated with either 1) estradiol (1 nM) or 2) estradiol (1 nM)+fulvestrant (100 nM). FIG. 10C shows immunofluorescent confocal micrographs of αvβ6 integrin (the known heterodimer of ITGB6) in MCF7:WS8 3D cultures treated with estradiol (1 nm) and with or without fulvestrant (100 nM) Scale bar=25 μm.

FIGS. 11A-11B show that the disclosed method enables discovery of a distinct intercellular proteome signature associated with therapy resistance in ER+ breast cancer cells. FIG. 11A shows a Venn diagram of intercellular proteins upregulated in response to 100 nM tamoxifen (log 2-fold change>1, FDR<0.05) in therapy sensitive (MCF7:WS8) or therapy resistant (TAMR) cells derived from the same MCF7:WS8 parental line. Differential protein expression was determined by PEA quantification of MCF7 3D cultures subjected to DBCO-PEG4-Desthiobiotin click labeling, full-length protein elution, and PEA-quantification. FIG. 11B shows a volcano plot of DBCO-PEG4-Desthiobiotin click and PEA-based differential intercellular protein expression in MCF7:WS8 cells and TAMR cells treated with estradiol (1 nM) and tamoxifen (100 nM).

FIG. 12 shows that DBCO-PEG4-Desthiobiotin click labeling and PEA-based quantification enables profiling of diverse cancer subtypes. Hierarchical clustering analysis of the top 100 proteins with the strongest evidence for differential expression across small-cell lung cancer lines (DMS53, H1048), patient derived xenograft triple-negative breast cancer organoids, HCC70 triple-negative breast cancer cell line, and ER+ breast cancer cell lines (MCF7:W S8, TAMR). Relative protein abundance was determined by DBCO-PEG4-Desthiobiotin labeling in 3D culture with 2% EHS matrix, streptavidin bead capture, full-length protein elution, and PEA-based quantification.

FIGS. 13A-13B show that mutant t-RNA synthetases, in conjunction with DBCO-PEG4-Desthiobiotin labeling, enable mutant-specific click labeling of intercellular proteins. FIG. 13A shows fluorescent micrographs of MCF7:WS8 cells expressing either a wild-type methionine t-RNA synthetase or a mutant (L274GMmMetRS) t-RNA synthetase engineered to recognize azidonorleucine. Cells were cultured in 3D 2% EHS matrix culture, treated with azidonorleucine (500 pM), labeled with DBCO-PEG4-Desthiobiotin, then fixed and stained with streptavidin-alexafluor568. Nuclei were counterstained with DAPI. Scale bar=10 μm. FIG. 13B shows the quantification of immunofluorescent micrographs from FIG. 13A. Values are represented as the integrated streptavidin-568 fluorescence intensity, normalized to threshold DAPI area, and averaged across replicates. Error bars represent the standard error of the mean. *** indicates p-value<0.001 as determined by an unpaired two-sided t-test.

### DETAILED DESCRIPTION

### I. Overview

The present disclosure provides methods for isolating full-length intercellular proteins from a complex, multicellular environment, including methods for detecting and quantifying the isolated proteins. The disclosed methods provide for isolation of the pericellular proteome or a targeted subset thereof such as, e.g., lipidated proteins, from complex cell culture systems (both in vitro and ex vivo, including, e.g., three-dimensional (3D) organotypic culture models) as well as from in vivo animal models. The methods harness bioorthogonal chemical reactions (e.g., click chemistry) to bypass non-target cells and factors (e.g., exogenous factors in a complex culture system, or non-target tissues in an in vivo model), infiltrate intercellular spaces, and retrieve the endogenous intercellular proteome. Moreover, the methods can be applied to cells representing diverse phenotypes and tissues, including unpassaged organoids from primary tissue, and allow for interrogation of the intercellular proteome in response to different stimuli such as, for example, different signals and factors relevant to disease development, progression, and treatment. Importantly, the disclosed methods allow for the capture of full-length proteins, including cell membrane-bound and soluble factor, and therefore can be integrated with a broad range of downstream analytic techniques, including LC/MS and immunoaffinity-based assays (e.g., Western blots, ELISA, antibody arrays, and Proximity Extension Assay (PEA)). As shown in the working examples herein, the disclosed methods enable discovery and downstream quantification of proteins that were undetectable using conventional LC/MS of crude lysates, and the methods can identify candidate proteins as therapeutic targets for drug development.

To target the pericellular proteome, the disclosed methods generally utilize first and second chemical groups that are reactive together in a bioorthogonal chemical reaction, wherein cells—cultured in vitro/ex vivo or grown in vivo—incorporate an amino acid analog or an alkanoic acid analog functionalized with the first chemical group into newly synthesized proteins. A cell-membrane-impermeable probe, comprising the second chemical group linked to a biotin analog moiety (e.g., a desthiobiotin, N3′-ethyl biotin, or 2-iminobiotin moiety) via a hydrophilic linker, is then used to label newly synthesized proteins that are pericellularly localized via a bioorthogonal chemical reaction between the first and second chemical groups. The probe allows for (a) labeling of live multicellular systems (in vitro, ex vivo, or in vivo), (b) infiltration into intercellular (e.g., nanolumenal) spaces, (c) specific labeling of pericellular proteins and not intracellular proteins, and (d) capture and elution of full-length proteins via a mild elution process. The labeled, pericellular proteins are isolated using a biotin-binding protein (e.g., streptavidin) immobilized on a solid support. The isolated proteins can then be eluted by, for example, competitive elution with free biotin or an analog thereof having a similar affinity for the biotin-binding protein.

The disclosed methods provide certain advantages over several existing technologies that have been conventionally used for pericellular proteomics. For example, because the disclosed methods capture full-length proteins, the methods are compatible with a wide range of downstream protein detection and quantification methods, including LC/MS, ELISA, Western blot, and the Proximity Extension Assay (PEA, e.g., using Olink® platforms and target panels). In addition, compared to NHS-ester based cell surface labeling (e.g., sulfo-NHS biotin), the methods of the present disclosure do not react with exogenous proteins (e.g., exogenous extracellular matrix (ECM) proteins present in ECM-based hydrogel culture systems; see, e.g., Example 1, infra, which demonstrates that in 3D organotypic culture, NHS-ester-based technology is unable to penetrate into intercellular spaces, due to excessive reactivity with Matrigel on the exterior). Further, unlike subcellular fractionation techniques using detergents or ultracentrifugation, the present methods capture both membrane-bound and soluble proteins that are present in intercellular spaces, allowing for quantification of both receptors and ligands. Still further, unlike proximity ligation-based biotinylation technologies, which produce a nearly irreversible non-covalent bond between biotin and streptavidin during capture, the present methods allow for the elution of full-length proteins and does not require on-bead proteolytic digestion for downstream analysis. Also, because full-length protein capture allows for downstream quantification with, e.g., calibrated immunoaffinity-based assays, the disclosed methods enable downstream absolute quantification of pericellular protein abundance.

Moreover, studies described herein demonstrate that the methods of the present disclosure can detect pericellular proteins that are not detectable by LC/MS (see Example 1, infra). For example, compared to LC/MS of crude lysates (using Orbitrap technology), when the disclosed method was used in conjunction with downstream PEA quantification, the method detected over 1,600 intercellular proteins that were not detectable by LC/MS (see id.).

The studies described herein also show that the present methods can identify new therapeutic target candidates that could not be identified using existing methods, and that the methods are generalizable to distinct cell and tissue types. For example, these studies demonstrate the isolation, identification, and quantification of intercellular proteins in cell line models of estrogen-receptor positive (ER+) breast cancer, triple negative breast cancer, and two models of small-cell lung cancer using the disclosed methods, including the identification of intercellular therapeutic target candidates in ER+ breast cancer. See Examples 2 and 3, infra. Studies also demonstrate that the disclosed methods quantify intercellular proteins in patient-derived xenograft organoid models.

### II. Methods of Isolating Intercellular Proteins In Vitro or Ex Vivo

In one aspect, the present disclosure provides a method for isolating proteins that are pericellular localized in a multicellular system in vitro or ex vivo. In some embodiments, the method generally includes the following steps: (a) culturing a plurality of cells in a complex cell culture system, wherein the plurality of cells are cultured in the presence of an amino acid analog functionalized with a first chemical group that is reactive in a bioorthogonal chemical reaction, and wherein the amino acid analog is incorporated into newly synthesized proteins; (b) contacting the culture system with a cell-membrane-impermeable probe having the formula A-L-D, wherein A is a molecule comprising a second chemical group that is reactive in a bioorthogonal chemical reaction, wherein the first chemical group is reactive with second chemical group, L is a hydrophilic linker, and D is a molecule comprising a biotin analog moiety capable of specifically binding to streptavidin with a dissociation constant greater than about 1 pM and an off-rate kinetic constant greater than about 1×10⁻³ l/s, and wherein newly synthesized proteins that are pericellularly localized are labeled with the probe by a bioorthogonal chemical reaction between the first and second chemical groups; (c) lysing the cultured cells to generate a lysate containing the labeled proteins and other cellular and extracellular components; (d) contacting the lysate with a capture reagent comprising a biotin-binding protein immobilized on a solid support, wherein the biotin-binding protein binds to the biotin analog moiety on the labeled proteins to yield a [labeled protein]:probe complex; and (e) separating the solid support from the lysate to separate the labeled proteins from the other cellular and extracellular components, thereby isolating the proteins that are pericellularly localized in the multicellular system.

In certain embodiments of the method, the complex cell culture system is three-dimensional (3D) cell culture system. A variety of different 3D cell culture systems are known in the art and may be used for in vitro or ex vivo culture of a multicellular system in accordance with the present disclosure. See generally, e.g., Kapalczynska et al., Arch. Med. Sci. 4:910-919, 2018. The use of 3D cell culture systems is particularly advantageous for interrogating intercellular signaling in accordance with the present disclosure. For example, 3D cell culture systems can allow cells to not only mimic the natural structure of a tissue, but to preserve more physiologically relevant cell-cell and cell-extracellular environment interactions, gene expression profiles, and cellular topology and biochemistry. See, e.g., Griffith and Schwartz, Nat. Rev. Mol. Cell Biol. 7:211-24, 2006; Bissell et al., Curr. Opin. Cell Biol. 15:753-762, 2003; Gilbert et al., Science 329:1078-1081, 2010; Engler et al., Cell 126:677-689, 2006; Lee et al., Tissue Eng. Part B Rev. 14:61-68, 2008; Birgerstodder et al., Semin. Cancer Biol. 15:405-412, 2005; Li et al., Cancer Res. 66:1990-1999, 2006; Fuchs et al., Cell 116:769-778, 2004; Gomez-Lechon et al., J. Cell Physiol. 77:553-562, 1998; Ghosh et al., J. Cell. Physiol. 204:522-531, 2005; Berthiaume et al., FASEB 10:1471-1484, 1996; Semino et al., Differentiation 71:262-70, 2003; Powers et al., Tissue Eng. 8:499-513, 2002.

In some variations, the 3D culture system comprises a medium containing a substance with gelling properties (a concentrated medium or gel-like substance). Suitable 3D cell culture media containing a substance with gelling properties are well-known in the art and include, e.g., hydrogels such as soft agar or multiprotein hydrogels. See, e.g., Sanyal, Corning 9:1-18, 2014; Krishnamurthy and Nör, Head Neck 35:1015-1021, 2015; Weiswald et al., Neoplasia 17:1-15, 2015; Li et al., J. Biomol. Screen 16:141-54; 2011; Sodunke et al., Biomaterials 28:4006-4016, 2007; Friedrich et al., Nat. Protoc. 4:309-324, 2009; Kleinman and Martin, Semin. Cancer Biol. 15:378-386, 2005; Dawson et al., Adv. Drug Deliv. Rev. 60:215-228, 2008; Kim, Semin. Cancer Biol. 15:365-377, 2005. Particularly suitable multiprotein hydrogels include extracellular matrix (ECM)-based hydrogels. Such ECM-based hydrogels contain at least one and more typically two or more (e.g., three or four) ECM proteins selected from laminin (e.g., laminin B1, laminin C1), collagen (e.g., collagen type I, collagen type IV), nidogen (also called entactin), perlecan (heparan sulfate proteoglycan), hyaluronic acid, vitronectin, and/or elastin). In some such embodiments, an ECM-based hydrogel is a hydrogel derived from Engelbreth-Holm-Swarm (EHS) mouse sarcoma such as, for example, Matrigel® matrix (Corning), Cultrex® BME (R&D Systems), or Geltrex™ matrix (Thermo Fisher Scientific). ECM-based hydrogels are particularly suitable for facilitating three-dimensional interactions of cells with the local environment and the formation of tissue-like structures and, in the context of tumor cells, can be used to study the aggressiveness of cells and their potential for metastasis. See Kapalczynska et al., Arch. Med. Sci. 4:910-919, 2018.

In other variations, the 3D culture system comprises a suspension culture on, e.g., a non-adherent surface. See generally, e.g., Kapalczynska et al., Arch. Med. Sci. 4:910-919, 2018. See also, e.g., Weiswald et al., Neoplasia 17:1-15, 2015; Li et al., J Biomol. Screen 16:141-54; 2011; Krishnamurthy and Nör, Head Neck 35:1015-1021, 2015. 3D suspension culture allows for simplicity and speed in conducting cell culture and ease in extracting cells from the medium. 3D suspension culture systems can be used, for example, as a model in studies for increasing the population of cancer-initiating cells. See, e.g., Kapalczynska et al., supra. Krishnamurthy and Nör, supra.

In yet other embodiments, the 3D culture system is a culture on a scaffold. The scaffold material is typically a fiber or fiber-like and made of a biodegradable material, which may be natural or synthetic. Suitable scaffold materials include silk, alginate, and extracellular matrix proteins such as, e.g., collagen or laminin. Using a scaffold-based culture system, cells can migrate among fibers and attach to the scaffold, fill the space among fibers, and grow and divide. See Kapalczynska et al., supra. 3D culture systems on a scaffold are also generally well-known. See, e.g., Sanyal, Corning 9:1-18, 2014; Lee et al., Tissue Eng. Part B Rev. 14:61-68, 2008; Jastrzebska et al., Rep. Pract. Oncol. Radiother. 20:87-98, 2014; Glicklis et al., Biotechnol. Bioeng. 67:344-353, 2000; Tan et al., Tissue Eng. 7:203-210, 2001; Sourla et al., Anticancer Res. 16:2773-2780, 1996; Justice et al., Drug Discov. Today 14:102-107, 2009; Cheng et al., Biomaterials 25:3211-3222, 2004; Stevens and George, Science 310:1135-1138, 2005; Curtis and Wilkinson, Biochem. Soc. Symp. 65:15-26, 1999; Hayrock, Methods Mol. Biol. 695:1-15, 2011.

In certain embodiments, a 3D cell culture system is selected from an ECM-based hydrogel; an agarose hydrogel (e.g., an agarose hydrogel that is used to form mammospheres); a methyl cellulose semi-solid media; a polyethylene glycol hydrogel formed by chemical cross-linking of multi-arm PEG polymers; a hydrogel formed by physical or chemical cross-linking of chemically modified forms of any one of the following polymers: hyaluronic acid, poly(lactic acid), alginate, chitosan, dextran, polyacrylamide; a hydrogel formed by engineered self-assembling peptides that form nanofibers (e.g., Corning® PuraMatrix™ Peptide Hydrogel); a hydrogel formed by electrospun fibers (e.g., methacrylated dextran); a slice culture of primary tissue (e.g., wherein primary tissues are mechanically dissected into thin slices ranging from 10 microns to 1 mm in thickness); a hanging drop spheroid culture; a spheroid cultures obtained using a micro-well array; and an air-liquid interface culture.

3D hydrogel systems are particularly suitable for use in accordance with the present methods. In the most general case of these systems, cells are cultured within a hydrogel that is produced by chemically or physically cross-linking synthetic, natural, or semisynthetic polymers using a cross-linking method that is orthogonal to the bioorthogonal chemical reaction, and where the polymer backbone bears only functional groups that are orthogonal to the bioorthogonal chemical reaction. Suitable orthogonal cross-linking reactions include, for example, thiol-ene Michael addition reactions between thiols and maleimides, reactions between primary amines and aldehydes, reactions between hydrazines or hydrazides and aldehydes, inverse electron demand diels alder reactions between transcyclooctenes and tetrazines (e.g., provided the bioorthogonal chemical reaction uses DBCO, and not BCN), cross-linking of methyacrylate by free-radical small-molecule cross-linkers, cross-linking of primary amines with bifunctional N-Hydroxysuccinimide (NHS) esters, and physical cross-linking with guest-host type interactions involving cyclodextrins. Suitable functional groups on the polymer backbone that would not interfere with the bioorthogonal labeling include amines (primary, secondary, and tertiary), amides, carbonyl, carboxyl, aldehyde, hydroxyl, thiols, alkenes, sulfate, phosphate, phenyl groups, ethers, esters, thioethers, and thioesters.

In some embodiments comprising the use of a 3D hydrogel system, cells are fully embedded in a hydrogel such as, e.g., an ECM-based hydrogel. In other variations, cells are cultured as a suspension in a mixture of the hydrogel-forming substance (e.g., an ECM-based extract used to form ECM-based hydrogels such as, for example, a matrix derived from Engelbreth-Holm-Swarm (EHS) mouse sarcoma) and an aqueous culture medium. For example, in a specific embodiment, cells are cultured in a 2% EHS matrix suspension media obtained by combining EHS matrix (e.g., Cultrex reduced growth factor basement membrane extract, R&D Systems #3433-005-01) with an aqueous culture medium (e.g., an RPMI culture medium) at a ratio of 1:49.

Cells (e.g., spheroids or organoids) are cultured in the complex culture system in the presence of the functionalized amino acid analog. As noted above, the amino acid analog is functionalized with a first chemical group that is reactive in a biorthogonal chemical reaction with a second chemical group that is present in the cell-membrane impermeable probe. Complementary pairs of chemical groups that are reactive in a bioorthogonal chemical reaction are generally well-known and may be used in accordance with the present disclosure. See, e.g., Saleh et al., J Biol. Eng. 13:43, 2019. Suitable bioorthogonal chemistries for use with the disclosed methods include bioorthogonal click chemical reactions such as, for example, 1,3-dipolar cycloaddition between a strained alkyne and an azide to form a triazole, a Staudinger ligation between an azide and a phosphine, a reaction of an oxanorbornadiene and an azide to from a triazole, an inverse-demand Diels-Alder reaction of a tetrazine (e.g., dipyridyl tetrazine) and a trans-cycloctyne, an inverse-demand Diels-Alder reaction of a tetrazine (e.g., monoaryl tetrazine) and a norbomene, a reaction of a tetrazine and a cyclopropene, a reaction of a cyclopropene and a nitrile imine, a photoinduced 1,3-dipolar cycloaddition of a tetrazole and an alkene, a 1,3-dipolar cycloaddition of a nitrile oxide and a norbomene, a [4+1]cycloaddition of a isocyanide and a tetrazine, or a 1,3-dipolar cycloaddition of a nitrone and a strained alkyne. In certain embodiments, complementary pairs of first and second chemical groups for use with the methods disclosed herein are selected from the above pairs ofcchemical groups.

A particularly suitable complementary pair of bioorthogonal chemical groups for use in accordance with the present methods is a strained alkyne and an azide. Accordingly in some variations, the functionalized amino acid analog comprises a first chemical group that is an azide. Particularly suitable azide-containing amino acid analogs for use in accordance with the present disclosure include azidohomoalanine, azidonorleucine, p-azido-Lphenylalanine, and 3-azido-L-tyrosine (see Table 1 below).

TABLE 1
Exemplary Azide-containing Amino Acid Analogs

| | | Corresponding | |
|---|---|---|---|
| Amino Acid | Codon | Natural Amino | |
| Analog | Replacement | Acid | Genetic Modification |
| Azidohomoalanine | AUG | Methionine | Not needed |
| Azidonorleucine | AUG | Methionine | Transgenic cell lines that |
| | | | express a mutant MetRS |
| | | | (methionyl-tRNA synthetase) |
| | | | capable of charging |
| | | | azidonorleucine |
| p-azido-L- | UUC, UUU | Phenylalanine | Transgenic cell lines that |
| phenylalanine | | | express a mutant PheRS |
| | | | (phenylalany1-tRNA |
| | | | synthetase) capable of |
| | | | charging p-azido-L- |
| | | | phenylalanine |
| 3-azido-L-tyrosine | UAC, UAU | Tyrosine | Transgenic cell lines that |
| | | | express a mutant TyrRS |
| | | | (tyrosyl-tRNA synthetase) |
| | | | capable of charging 3-azido- |
| | | | L-tyrosine |

The functionalized amino acid analog is added to the culture system media in a sufficient quantity to allow incorporation of the amino acid analog into newly synthesized proteins. In typical variations, adding the amino acid analog to the culture system media includes replacing the media in which the cells are grown with media containing the amino acid analog. Typically, the media containing the amino acid analogue will have a reduced concentration of the corresponding natural amino acid (e.g., reduced methionine or methionine-free if using a methionine analog such as, for example, azidohomoalanine). Concentrations of the functionalized amino acid analog in the culture media typically range from about 0.05 mM to about 50 mM, from about 0.05 mM to about 10 mM, or from about 0.1 mM to about 10 mM. More typically, concentrations of the functionalized amino acid analog in the culture media range from about 0.05 mM to about 2 mM, from about 0.1 mM to about 2 mM, or from about 0.1 mM to about 1 mM. In a specific variation, the concentration of the functionalized amino acid analog is about 0.5 mM. The optimal dose will be dependent upon the intrinsic protein synthesis rate of the biological system. In embodiments comprising replacing the culture system media with the media containing the amino acid analogue, cells may be washed to remove excess amounts of the corresponding natural amino acid (e.g., excess methionine if using a methionine analog) before adding the replacement media. For example, cells (e.g., cells cultured as spheroids in a suspension media such as, for example a 2% EHS matrix suspension media) may be washed by centrifuging the cells (e.g., 200×g for 3 minutes), removing the supernatant, and resuspending the cells in a suitable buffered saline (e.g., PBS) before resuspending cells in the media containing the amino acid analog. In other variations comprising culture of 3D-embedded organoids, organoids may be washed, for example, in organoid media that is free of the corresponding natural amino acid (e.g., 2 to 4 times for about 10 minutes each at 37° C. with no agitation).

In some embodiments, substitution of the functionalized amino acid analog for its corresponding natural amino acid in protein synthesis occurs because the corresponding tRNA synthetase is sufficiently promiscuous to allow misacylation of the tRNA synthetase with the amino acid analog (e.g., methionyl-tRNA synthetase is sufficiently promiscuous to allow misacylation of methionine tRNA with azidohomoalanine). In other embodiments discussed further herein, a genetically engineered cell line expresses a mutant tRNA synthetase that selectively incorporates the functionalized amino acid analog (e.g., a mutant MetRS that selectively incorporates azidonorleucine). After sufficient time, generally from about 30 minutes to about 24 hours or more, the amino acid analog is assimilated by the cells and incorporated into protein synthetic pathways, whereby the amino acid analog is incorporated into newly synthesized proteins.

Following incorporation of the amino acid analog into newly synthesized proteins, newly synthesized proteins that are pericellulary localized are labeled with the cell-membrane-impermeable probe having the formula A-L-D in a bioorthogonal chemical reaction between the first bioorthogonal chemical group of the amino acid analog and the second bioorthogonal chemical group of probe, thereby functionalizing the pericellular proteins with the biotin analog moiety of the probe.

In certain variations, the bioorthogonal chemical reaction is a copper-free bioorthogonal click chemistry reaction between a strained alkyne and an azide to form a triazole. Accordingly, in certain embodiments, the amino acid analogue comprises a first chemical group that is an azide and the cell-membrane-impermeable probe having the formula A-L-D comprises a second chemical group that is a strained alkyne. Molecules comprising a strained alkyne and which are suitable for use in 1,3-dipolar cycloaddition click reactions with azides are generally well-known in the art. Particularly suitable strained alkynes include cyclic alkynes such as, e.g., cyclooctynes. Thus, in certain variations, A comprises a cyclooctyne, which may be, for example, a monocyclic or bicyclic, unsubstituted or substituted cyclooctyne (e.g., a monofluorinated or difluorinated cyclooctyne), an unsubstituted or substituted monoarylcyclooctyne (e.g., a monobenzocyclooctyne), or an unsubstituted or substituted diarylcyclooctyne (e.g., a dibenzocyclooctyne). In some embodiments, A comprises a cyclooctyne selected from dibenzocyclooctyne (DIBO/DBCO), OCT cyclooctyne, monofluorinated cyclooctyne (MOFO), difluorocyclooctyne (DIFO), dimethoxyazacyclooctyne (DIMAC), dibenzoazacyclooctyne (DIBAC), biarylazacyclooctynone (BARAC), bicyclononyne (BCN), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), and aryl-less octyne (ALO).

In some embodiments, A is a cyclic alkyne comprising the formula A1

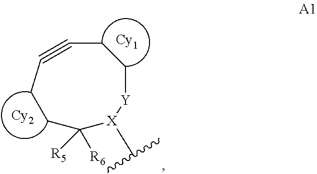

wherein:
   X is CH or N;
   Cy1 is absent, phenyl, pyridyl, or pyrrolyl, and is optionally substituted with 1, 2, 3, or 4 substituents selected from the group consisting of hydrogen, halogen, C1-C3 alkyl, C1-C3 alkoxy, C1-C3 carboxyalkyl, and sulfonyl;
   Cy2 is absent, phenyl, pyridyl, or pyrrolyl, and is optionally substituted with 1, 2, 3, or 4 substituents selected from the group consisting of hydrogen, halogen, C1-C3 alkyl, C1-C3 alkoxy, C1-C3 carboxyalkyl, and sulfonyl; and
   R5 and R6 are independently selected from the group consisting of hydrogen, halogen, C1-C3 alkyl, C1-C3 alkoxy, C1-C3 carboxyalkyl, sulfonyl, or together form an oxo.

In certain of these embodiments, Cy1 is absent, phenyl, pyridyl, or pyrrolyl, and is optionally substituted with 1, 2, 3, or 4 substituents selected from the group consisting of hydrogen, fluoro, chloro, bromo, iodo, methyl, ethyl, methoxy, ethoxy, carboxy methyl, and sulfonyl; Cy2 is absent, phenyl, pyridyl, or pyrrolyl, and is optionally substituted with 1, 2, 3, or 4 substituents selected from the group consisting of hydrogen, fluoro, chloro, bromo, iodo, methyl, ethyl, methoxy, ethoxy, carboxy methyl, and sulfonyl; and R5 and R6 are independently selected from the group consisting of hydrogen, fluoro, chloro, bromo, iodo, methyl, ethyl, methoxy, ethoxy, carboxy methyl, and sulfonyl, or together form an oxo.

In certain of above embodiments, X is N, and/or Cy1 and Cy2 are phenyl.

In representative embodiments of a cyclic alkyne comprising the formula A1, the cyclic alkyne comprises the formula A2

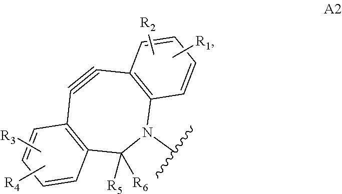

wherein R1, R2, R3, R4, R5, and R6 are independently selected from the group consisting of hydrogen, fluoro, chloro, bromo, iodo, methyl, methoxy, carboxy methyl, and sulfonyl.

In more particular variations of a cyclic alkyne having the formula A1 as above, the cyclic alkyne is selected from the group consisting of dibenzocyclooctyne (DIBO/DBCO), OCT cyclooctyne, monofluorinated cyclooctyne (MOFO), difluorocyclooctyne (DIFO), dimethoxyazacyclooctyne (DIMAC), dibenzoazacyclooctyne (DIBAC), biarylazacyclooctynone (BARAC), bicyclononyne (BCN), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), and aryl-less octyne (ALO).

In some variations, the cyclic alkyne is dibenzocyclooctyne (DIBO/DBCO) (e.g., dibenzocyclooctyne-amine). Accordingly, in certain embodiments, A is a molecule comprising the formula A3

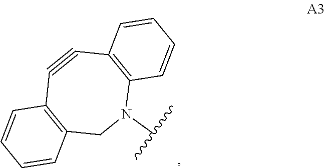 [SMILES: *N1Cc2ccccc2C#Cc2ccccc21]

wherein [symbol] designates a point of attachment. In some such embodiments, A has the formula A4

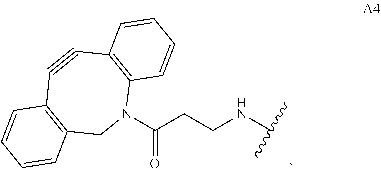 [SMILES: *NCCC(=O)N1Cc2ccccc2C#Cc2ccccc21]

wherein [symbol] designates a point of attachment.

The probe's hydrophilic linker (L, joining A and D) confers cell membrane impermeability to the probe. The hydrophilic linker typically has an overall neutral or negative charge and is of sufficient length to ensure that the probe does not cross the cell membrane so that intracellular proteins are not labeled substantially. In typical variations, the hydrophilic linker is a hydrophilic polymer comprising a plurality of monomeric residues (e.g., at least four monomeric residues) that are non-charged or negatively charged. Particularly suitable non-charged monomeric residues include, e.g., polyethylene glycol (PEG) monomeric residues (i.e., ethylene oxide repeat units), polyvinyl alcohol (PVA) monomeric residues, and polyvinylpyrrolidone (PVP) monomeric residues. In certain embodiments, the hydrophilic linker is a PEG linker comprising from four to 16 or from four to 12 PEG monomeric residues. In some such variations, the hydrophilic linker comprises the formula L1

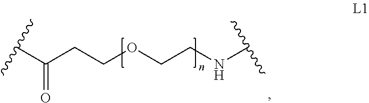

wherein n is an integer from 4 to 16, and [symbol] des nates a point of attachment. In some such embodiments, n is an integer from 4 to 12, from 4 to 10, from 4 to 8, or from 4 to 6; in more specific variations, n is 4. In other embodiments of a hydrophilic linker comprising a plurality of non-charged monomeric residues, the linker comprises from four to 16 (e.g., from 4 to 12, from 4 to 10, from 4 to 8, from 4 to 6, or 4) PVA or PVP monomeric residues.

Suitable negatively charged hydrophilic linkers include hydrophilic polymers comprising at least one negatively charged residue having an anionic group selected from a sulfonate, a phosphate, a phosphonate, and a carboxylic acid. For example, in some embodiments, the hydrophilic linker comprises at least one sulfonate. In some such embodiments, the hydrophilic linker comprises the formula L2 or L3

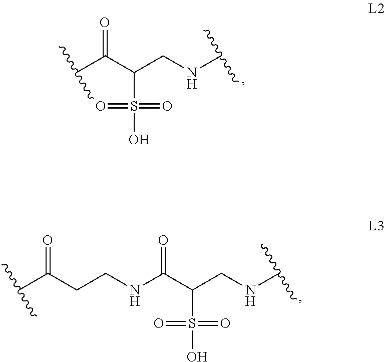 [SMILES: *NCC(C(*)=O)S(=O)(=O)O ; *NCC(C(=O)NCCC(*)=O)S(=O)(=O)O]

wherein [symbol] designates a point of attachment. In other variations of a negatively charged hydrophilic linker, the hydrophilic linker comprises a plurality of non-charged monomeric residues (e.g., a plurality of PEG, PVA, or PVP monomeric residues) and at least one negatively charged residue having an anionic group selected from a sulfonate, a phosphate, a phosphonate, and a carboxylic acid. In some such variations, the hydrophilic linker comprises the formula L4, L5, or L6

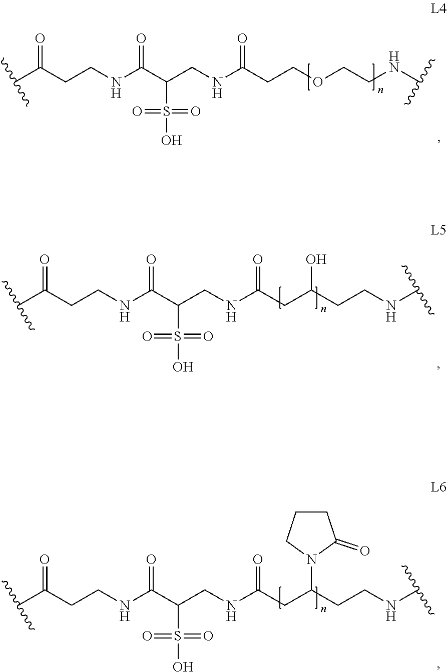

wherein n is an integer from 4 to 16, and [symbol] designates a point of attachment. In some such embodiments, n is an integer from 4 to 12, from 4 to 10, from 4 to 8, or from 4 to 6; in more specific variations, n is 4.

In yet other embodiments comprising a negatively charged hydrophilic linker, the linker is a hydrophilic polymer comprising a plurality of negatively charged monomeric residues, each having an anionic group selected from a sulfonate, a phosphate, a phosphonate, and a carboxylic acid.

The biotin analog moiety of the probe confers the ability to elute isolated pericellular proteins by competitive elution with free biotin (or an analog thereof having similar affinity for the biotin-binding protein). The ability to elute by competitive elution with free biotin is dependent on both thermodynamic and kinetic considerations. For example, in particular embodiments utilizing streptavidin as the biotin-binding protein, thermodynamic equilibrium heavily favors the biotin-streptavidin interaction over the [biotin analog]-streptavidin interaction. Typically, the kinetic off-rate constant for the [biotin analog]-streptavidin interaction is sufficiently large for the biotin analog moiety to be displaced on timescales of around 24 hours. Both constraints are typically satisfied simultaneously (and practically speaking are not fully independent). Table 2 below shows known affinities of biotin and two biotin analogs, desthiobiotin, and N3′-ethyl biotin, for streptavidin.

TABLE 2
Affinities of Biotin, Desthiobiotin, and N3′-ethyl Biotin for Streptavidin

| | Kₒₙ(1/M | | | |
|---|---|---|---|---|
| Biotin analog | 1/s) | K_{off} (1/s) | K_{d} (M) | Reference |
| Biotin | 6.7e7 | 4.4e−6 | 6.5e−14 | Magalhaes et al., Protein Sci. |
| | | | | 20:1145-1154, 2011 |
| Desthiobiotin | 2.7e7 | 1.8e−2 | 6.6e−10 | Magalhaes et al., Protein Sci. |
| | | | | 20:1145-1154, 2011 |
| N3-ethyl | 1.5e5 | 5.8e−2 | 8e−10 | Ying and Branchaud, Chem. |
| biotin | | | | Commun. 47:8593-8595, |
| | | | | 2011 |

From a thermodynamic standpoint, the dissociation constant of the biotin analog moiety should be substantially larger than biotin. Experimentally, prior experiments have shown that dissociation constants in the range of 0.1 to 1 nM are suitable for mild elution. In the context of streptavidin as the biotin-binding protein, in the limit of large excess biotin in comparison to the number of available streptavidin sites, it is expected that the ratio of [biotin bound to streptavidin] to [biotin analog bound to streptavidin] will approach the ratio of their affinity constants (or equivalently, the inverse ratio of their dissociation constants). Biotin analogs with dissociation constants as low as about 1 pM are particularly suitable with the present methods since this provides over 100 times greater affinity for biotin over the biotin analog moiety. In certain embodiments, the biotin analog moiety is capable of binding to streptavidin with a dissociation constant greater than about 5 pM, greater than about 10 pM, greater than about 50 pM, greater than about 100 pM, greater than about 300 pM, greater than about 500 pM, or greater than about 600 pM.

From a kinetic perspective, the off-rate of the binding reaction occurs on time-scales that allow for equilibrium to be reached in a reasonable time. The characteristic time-scale is approximately the inverse of the off-rate. For example, the biotin-streptavidin interaction has an unbinding time-scale (inverse of the off-rate) on the order of 1 million seconds, which corresponds to over 200 days. Typically, the unbinding should be nearly complete within 24 hours. Biotin analogs with off-rate kinetic constants as low as about 1×10⁻³ l/s are particularly suitable for use with the present methods. In certain embodiments, the biotin analog moiety is capable of binding to streptavidin with an off-rate kinetic constant greater than about 2×10⁻³ l/s, greater than about 3×10⁻³ l/s, greater than about 5×10⁻³ l/s, greater than about 1×10⁻² l/s, or greater than about 1.5×10⁻² l/s.

In certain embodiments, the biotin analog moiety capable of specifically binding to streptavidin with a dissociation constant greater than about 1 pM and an off-rate kinetic constant greater than about 1×10⁻³ l/s is derived from a biotin analog selected from desthiobiotin, N3′-ethyl biotin, and 2-iminobiotin. In particular variations, a desthiobiotin moiety, N3′-ethyl biotin analog moiety, or 2-iminobiotin moiety respectively has the formula D1, D2, or D3

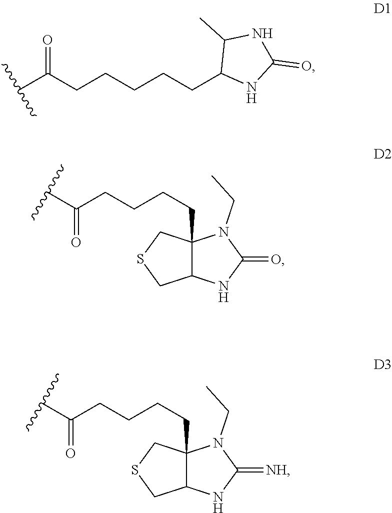 [SMILES: *C(=O)CCCCCC1NC(=O)NC1C ; *C(=O)CCCC[C@]12CSCC1NC(=O)N2CC ; *C(=O)CCCC[C@]12CSCC1NC(=N)N2CC]

wherein [symbol] designates a point of attachment.

In certain embodiments, the linker and biotin analog moiety portions of the probe (“L-D”) together have the formula [L-D]1 or [L-D]2

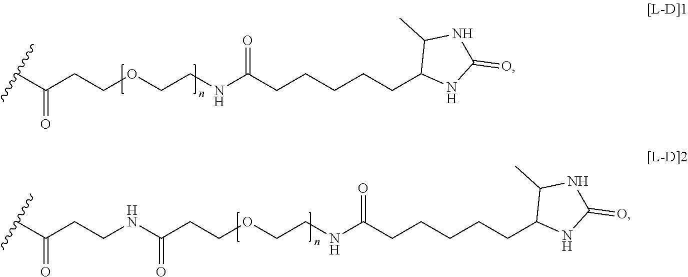

wherein n is an integer from 4 to 16, and [symbol] designates a point of attachment. In some such embodiments, n is an integer from 4 to 12, from 4 to 10, from 4 to 8, or from 4 to 6; in more specific variations, n is 4.

In certain embodiments wherein A comprises a cyclic alkyne, the cyclic alkyne and linker portions of the probe (“A-L”) together have the formula [A-L]1

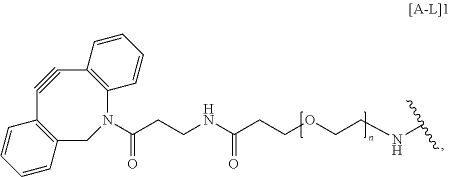

wherein n is an integer from 4 to 16, and [symbol] designates a point of attachment. In some such embodiments, n is an integer from 4 to 12, from 4 to 10, from 4 to 8, or from 4 to 6; in more specific variations, n is 4.

In particular embodiments of a probe comprising a desthiobiotin moiety, the probe has the formula P1, P2, or P3

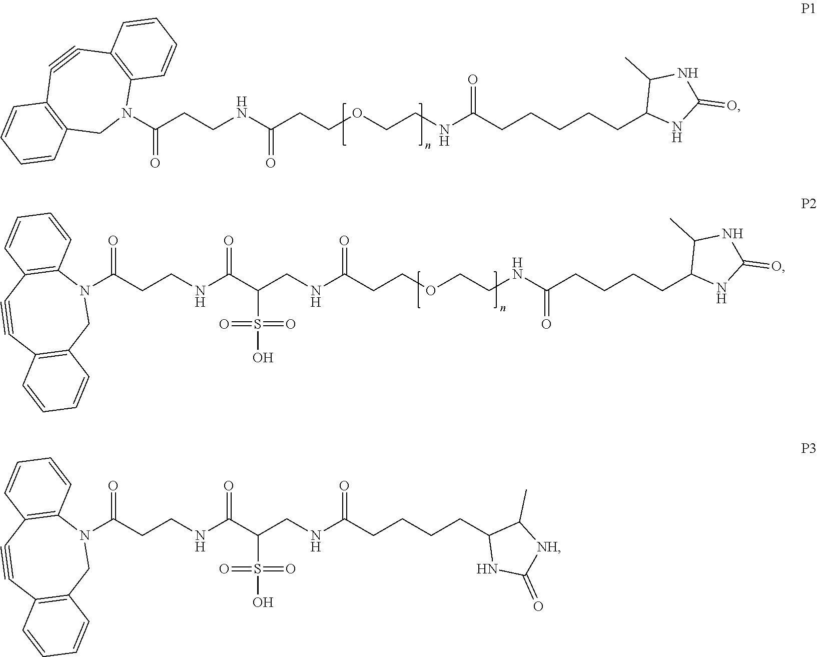

wherein n is an integer from 4 to 16. In some such embodiments of a probe having the formula P1 or P2, n is an integer from 4 to 12, from 4 to 10, from 4 to 8, or from 4 to 6 (e.g., n is 4). In some embodiments of a probe having the formula P1, P2, or P3, the desthiobiotin moiety is specifically the d-isomer and the probe comprises the formula P1a, P2a, or P3a

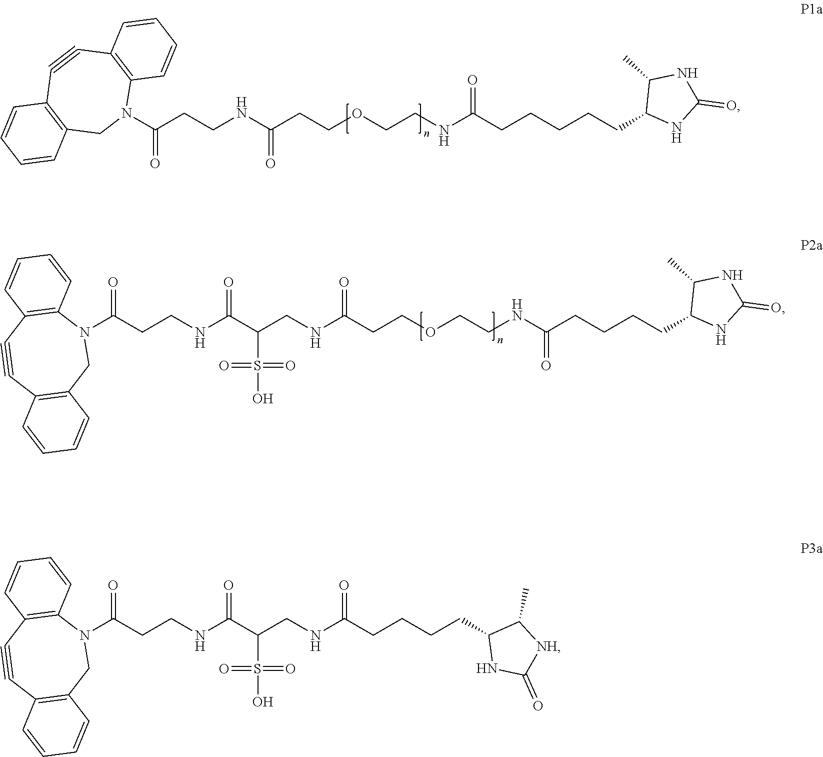

wherein n is an integer from 4 to 16 (e.g., from 4 to 12, from 4 to 10, from 4 to 8, from 4 to 6, or 4).

In other embodiments of a probe comprising a desthiobiotin moiety, the probe has the formula P4 or P5

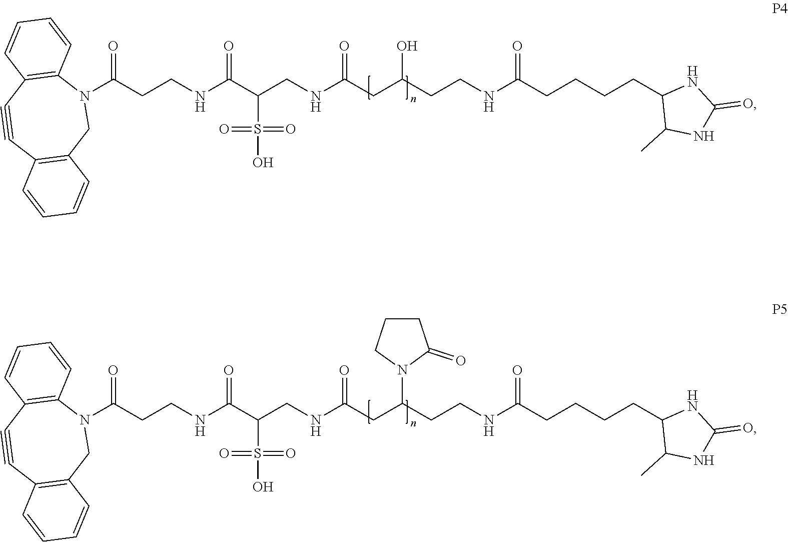

wherein n is an integer from 4 to 16. In some such embodiments, n is an integer from 4 to 12, from 4 to 10, from 4 to 8, or from 4 to 6 (e.g., n is 4).

In some specific variations, the probe has the formula P1₄.

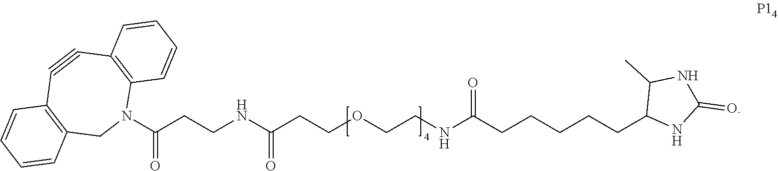

In some such embodiments, the desthiobiotin moiety is specifically the d-isomer and the probe comprises the formula P1a₄

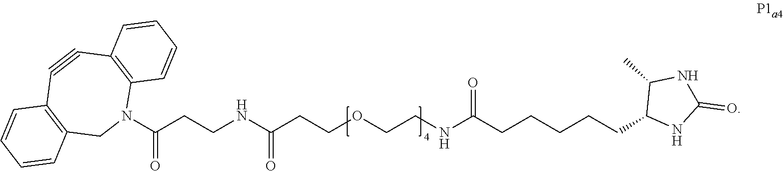

To label pericellular proteins with the cell-membrane-impermeable probe following incorporation of the amino acid analog into newly synthesized proteins, cultured cells (e.g., spheroids or organoids) are typically first washed to remove unincorporated amino acid analog. For example, cells (e.g., cells cultured as spheroids in a suspension media such as, for example a 2% EHS matrix suspension media) may be washed at least once, more typically two or three times, by centrifuging the cells (e.g., 200×g for 1 minute), removing the supernatant, and resuspending the cells in a suitable buffered saline (e.g., PBS). Washed cells can then be resuspended in a solution (e.g., PBS with 1% bovine serum albumin, or in culture media, which may be free of the natural amino acid corresponding to the amino acid analog) containing the probe at a suitable concentration. In other variations comprising culture of 3D organoids embedded, e.g., in a hydrogel, organoids may be washed, for example, in organoid media that is free of the corresponding natural amino acid (e.g., 2 to 4 times for about 30 minutes each at 37° C. with no agitation), and then the embedded organoids submerged in media (e.g., free of the corresponding natural amino acid) containing the probe at a suitable concentration. Concentrations of the probe typically range from about 0.001 mM to about 10 mM, from about 0.01 mM to about 10 mM, from about 0.03 mM to about 3 mM, or from about 0.1 mM to about 1 mM. More typically, concentrations of the probe range from about 0.01 mM to about 0.3 mM. In a specific variation, the concentration of the probe is about 0.3 mM. The optimal dose will be dependent upon the intrinsic protein synthesis rate of the biological system. Cells are incubated with the probe for a time sufficient to allow the probe to infiltrate intercellular spaces and label pericellular proteins via the bioorthogonal chemical reaction between the incorporated amino acid analog comprising the first chemical group (e.g., an azide) and the second chemical group (e.g., a strained alkyne) of the probe. For example, cells may be incubated with the probe from about 10 minutes to about one hour, from about 15 minutes to about one hour, from about 20 minutes to about 50 minutes, from about 20 minutes to about 45 minutes, or about 30 minutes. In typical variations, cells are incubated with the probe at a temperature of from about 4° C. to about 37° C. Lower temperatures with this range will reduce active internalization of the probe. Higher temperatures within this range will accelerate the reaction and improve the condition of the cells during the labeling period. For systems that have high amino acid analog incorporation rates, lower temperatures may provide a particularly suitable balance between localization specificity and signal. In some specific variations, cells are incubated with the probe at a temperature that is at or near the native temperature for the cell type(s) grown in the culture system (e.g., at about 37° C. for mammalian cells). In variations comprising culture of embedded 3D organoids, incubation with the probe solution may be performed with agitation (e.g., about 80 RPM on an orbital shaker). In some embodiments, an average of from one to about five probes are incorporated into newly synthesized pericellular proteins.

Following incubation with the probe, cells are typically washed to remove excess, unreacted probe. For example, cells (e.g., cells cultured as spheroids in a suspension media such as) may be washed at least once, more typically two to four times, by centrifuging the cells (e.g., 200×g for 1 minute), removing the supernatant, and resuspending the cells in buffered saline (e.g., PBS). In other variations comprising culture of 3D organoids embedded, e.g., in a hydrogel, organoids may be washed, for example, at least once in organoid media (e.g., ice-cold media, typically free of the natural amino acid corresponding to the amino acid analog), followed by at least one, more typically two or three, washes in a suitable buffered saline (e.g., ice-cold PBS); such washes may be performed, for example, for about 10 minutes at 4° C. with gentle orbital agitation.

Following labeling of cells with the cell-membrane-impermeable probe and any washing of excess, unreacted probe, cells are subjected to lysis to generate a lysate containing the labeled proteins and other cellular and extracellular components. Methods for lysing cells for subsequent biomolecular analysis are generally well-known in the art. See, e.g., Islam et al., Micromachines 8:83, 2017. Such methods may include for example, chemical lysis using, e.g., one or more detergents (also called surfactants), physical lysis using, e.g., osmotic shock or acoustic lysis (e.g., sonication), mechanical lysis (e.g., homogenization using a tissue homogenizer), enzymatic digestion using, e.g., tissue digestion enzymes such as, for example, collagenase, chondroitinase, or hyaloronidase to dissolve or disrupt extracellular structures not easily disrupted by other methods, and combinations thereof. Particularly suitable for lysing mammalian cells are chemical lysis methods using one or more detergents, which are effective in solubilizing hydrophobic molecules and capable of disrupting, e.g., the native lipid-lipid and lipid-protein interactions in cells. Generally, detergents are water-soluble surface-acting agents comprising a hydrophobic portion, typically a long alkyl chain, attached to a hydrophilic or water-solubility-enhancing functional group. Based on their charge carrying capacity, detergents may be cationic, anionic, zwitterionic, or non-ionic. Exemplary detergents include Triton-X-100, nonyl phenoxypolyethoxylethanol (NP-40), sodium dodecyl sulfate (SDS), or lithium lauryl sulfate (LLS). Typically, a lysing reagent containing one or more detergents will contain a suitable buffer (e.g., Tris) and may further contain a salt (e.g., NaCl), a chelating agent (e.g., EDTA), protease inhibitors, and/or phosphatase inhibitors. In a specific embodiment, a lysing reagent contains 0.1% SDS, 1% Triton-X 100, 150 mM NaCl, 20 mM Tris, 1 mM EDTA, pH 7.4, supplemented with protease inhibitors (e.g., Halt Protease inhibitor cocktail, 100× dilution; Thermo Fisher Scientitic) and phosphatase inhibitors (e.g., Halt Phosphatase inhibitor cocktail, 100× dilution; Thermo Fisher Scientific). To lyse cells using a detergent-based lysing reagent, probe-labeled cells are contacted with the lysing reagent and incubated in the reagent for a time sufficient to solubilize cell membrane components. For example, labeled cells from a suspension culture may be resuspended in the lysing reagent and incubated, e.g., for about one hour at 4° C. In other variations comprising culture of embedded 3D organoids, organoids (e.g., in hydrogel domes) may be collected into the lysing reagent (for example, in a serial manner by pipetting the lysing reagent into each well and pipetting up and down) and incubated, e.g., for about one hour at 4° C. In some variations, sonication may be used to facilitate lysis. For example, in some embodiments, samples (e.g., cell suspensions or organoids lysed as above) are sonicated (using, e.g., a probe sonicator with 6 pulses, 0.5 seconds each) before and after the incubation period. Following lysis, cells are typically centrifuged (e.g., at 12,000×g for 10 minutes, 4° C.) to remove insoluble material, and the supernatant (representing the cleared cell lysate) is collected and used for the next step of enrichment.

To isolate the labeled proteins from the lysate, the lysate is contacted with a capture reagent comprising a biotin-binding protein (e.g., streptavidin or avidin) immobilized on a solid support, under conditions whereby the biotin-binding protein binds to the biotin analog moiety on the labeled proteins to yield a [labeled protein]:probe complex. In some embodiments, the solid support is a particulate solid support (e.g., beads). Suitable solid supports include, for example, silica solid supports such as, e.g., silica beads (e.g., silica microspheres), which may be contained within a column. In other variations, the solid support is a magnetic bead solid support (e.g., magnetic polystyrene beads). Following capture of the labeled proteins on the solid support, the solid support is separated from the lysate to separate the labeled proteins from other cellular and extracellular components. Separation of the support from other cellular and extracellular components can be accomplished by any appropriate technique such as, e.g., washing a support complexed with labeled proteins one or more times (e.g., two or three times) to remove other components. In embodiments using a particulate solid support, such as magnetic beads, particles complexed with the labeled proteins may be suspended in a washing solution and retrieved from the washing solution, in some embodiments by using magnetic attraction. For example, magnetic beads may be resuspended in the lysate containing the labeled proteins and the bead/lysate mixture incubated for, e.g., 1 to 3 hours at room temperature with gentle agitation (e.g., orbital agitation) to prevent the beads from settling, followed by separation of the magnetic beads removal of the supernatant (the “unbound fraction”), and washing of the beads (e.g., 3 times) by resuspending the beads in a wash buffer (e.g., a buffered saline such as a Tris-buffered saline) and then separating the magnetic beads.

Once the labeled pericellular proteins are isolated as above, the pericellular proteins may be eluted from the immobilized biotin-binding protein. Elution typically includes competitive elution with free biotin, or a biotin analog having a similar affinity for streptavidin as biotin (e.g., a biotin analog capable of specifically binding to streptavidin with a dissociation constant less than about 0.01 pM and an off-rate kinetic constant less than about 1×10⁵ l/s). Such competitive elution with free biotin or similar affinity biotin analog is particularly advantageous as the elution conditions are typically mild and allow for elution of full-length proteins. For example, to elute bound proteins from magnetic beads, the magnetic beads may be resuspended in an elution buffer containing biotin or the similar affinity biotin analog (e.g., a buffer containing 0.1% SDS, 1% Triton X-100, 150 mM NaCl, 20 mM Tris, 1 mM EDTA, 3 mg/mL biotin; about 100 μL of elution buffer is used per 100 μL of starting lysate material is suitable). As biotin free acid is only soluble in water in its deprotonated form, a concentrated form of the buffer (e.g., 2X) may be initially prepared and titrated with a suitable base (e.g., sodium hydroxide) until all biotin is fully dissolved and a suitable pH is reached (e.g., pH 7.4-7.6), and the resulting buffer solution then diluted with water as needed. In some specific variations of this elution method, the samples are first incubated for a shorter time period (e.g., about 2 hours) at a higher temperature (e.g., 37° C.) with agitation (e.g., 200 RPM), followed by a longer incubation period (e.g., about 16 hours) at a lower temperature (e.g., about 4° C.) with gentle orbital agitation. Following incubation in the elution buffer, magnetic beads are separated (e.g., on a magnetic stand) and the supernatant collected as the final elution fraction.

In further variations, a method in accordance with the present disclosure further includes analyzing one or more of the isolated proteins. In some embodiments, analyzing one or more of the isolated proteins comprises detecting the one or more proteins; in some such embodiments, the protein detection includes identifying the one or more proteins. In other, non-mutually exclusive embodiments, analyzing one or more of the isolated proteins comprises quantifying the one or more proteins. Various techniques for analyzing proteins are known and may be used in accordance with the present methods. Suitable analytical techniques include, for example, two-dimensional electrophoresis (2DE), liquid chromatography/mass spectrometry (LC/MS), liquid chromatography with tandem mass spectrometry (LC-MS/MS), nanoscale liquid chromatography with tandem mass spectrometry (nano LC-MS/MS), matrix assisted laser desorption ionization-time of flight mass spectrometry (MALDI-TOF MS), high-performance liquid chromatography (or high-pressure liquid chromatography) (HPLC), enzyme-linked immunosorbent assay (ELISA), Western blot analysis, antibody array analysis, and Proximity Extension Assay (PEA). Because proteins can be isolated in their full-length form in accordance with the disclosed isolation methods, the methods are particularly amenable to downstream immunoaffinity-based assays, including, for example, ELISA, Western blot analysis, antibody array analysis, and PEA. In certain specific variations of the disclosed method, the method further comprises detecting (e.g., identifying) and/or quantifying one or more of the isolated pericellular proteins using an analytic technique as above.

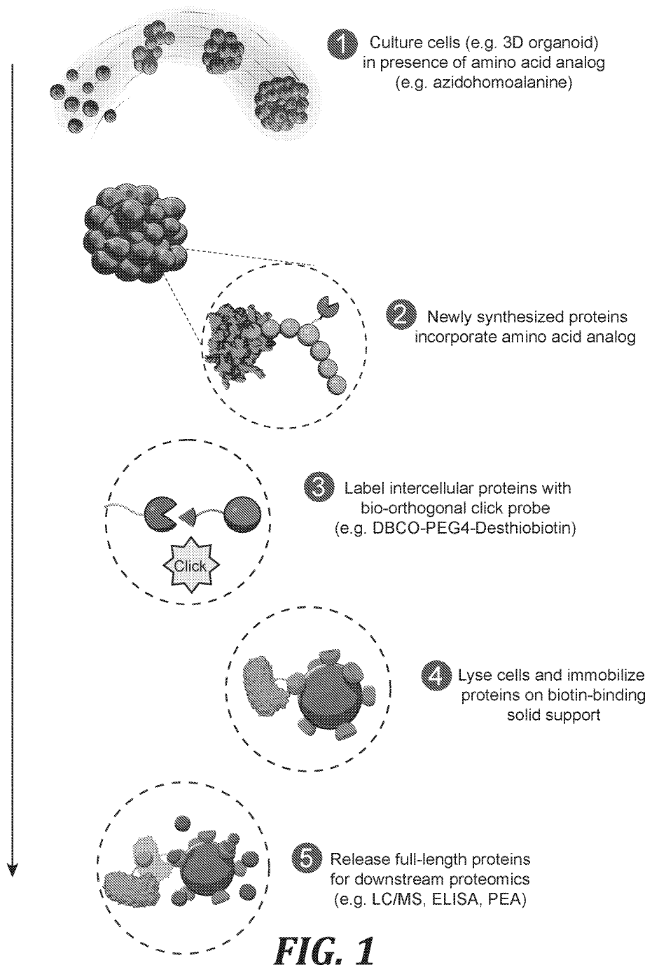

A particularly suitable immunoaffinity-based assay for detection and quantification of isolated proteins is Proximity Extension Assay (PEA) (Olink® Proteomics). PEA is used in exploratory protein analysis, as it offers exceptional sensitivity (e.g., sub-pg/ml) and specificity, while only consuming a minimum amount of sample (e.g., as low as 1 μL). PEA uses two matched antibodies per target antigen. Each antibody pair is labeled with unique DNA oligonucleotide-barcodes. When the antibodies bind to the same target protein in solution, the DNA oligonucleotide strands anneal with enough stability to enable DNA extension. The DNA barcode is then amplified. The resulting amplicons are measured by, e.g., quantitative Polymerase Chain Reaction (qPCR) or Next Generation Sequencing (NGS) for absolute or relative quantification. Thus, PEA combined with a library of protein markers (such as, for example, Olink® Explore 3072) can be used to analyze thousands of markers simultaneously. See, e.g., Wik et al., Mol. Cell Proteomics 20: 100168, 2021 (FIG. 1).

As indicated above, the Olink® Explore 3072 library is particularly suitable for use with PEA and certain specific variations of the disclosed method utilizing PEA for protein analysis. Olink® Explore 3072 is a library of ~3000 different protein assays to be used in proteomic studies. This library can be subdivided into categories: secreted proteins, organ specific blood biomarkers, inflammatory markers, approved and ongoing drug target proteins, and exploratory proteins. Additionally, the Olink® Explore platform comprises eight different panels, and these panels are organized by disease, e.g., cardiometabolic, oncology, etc. With the Explore 3072 panels, the resulting amplicons are sequenced and the readout of the analysis is NGS counts, which then represent the original protein concentration in the sample.

Proximity Extension Assay (PEA) and its use for detecting and quantifying proteins in biological samples are well-known and are further described, e.g., in U.S. Pat. Nos. 6,511,809; 9,677,131; 9,777,315; 10,731,206; and 10,781,473.

A range of different cell types may be used for isolation of a pericellular proteome in accordance with the methods disclosed herein. Particularly suitable cells for use in the methods include cells selected from fibroblasts, pericytes, epithelial cells, endothelial cells, neuronal cells, glial cells, lymphocytes, macrophages, dendritic cells, mast cells, hepatocytes, smooth muscle cells, skeletal muscle cells, cardiac muscle cells, adipocytes, osteocytes, osteoblasts, osteoclasts, melanocytes, mesangial cells, keratinocytes, germ cells (oocytes, spermatogonia), and progenitors thereof. In some embodiments, cells for isolation of a pericellular proteome comprise stem cells such as, e.g., hematopoietic stem cells or mesenchymal stem cells. In certain embodiments, cells for isolation of a pericellular proteome comprise hematopoietic cells, which may be selected from hematopoietic stem cells, neutrophils, CD4+ naïve T-cells, CD8+naïve T-cells, CD4+ Central Memory T-cells, CD8+ Central Memory T-cells, CD4+ Effector Memory T-cells, CD8+ Effector Memory T-cells, follicular helper T cells, regulatory T-cells, natural killer cells, naïve B-cells, memory B-cells, plasma cells, precursor B-cells, monocytes, dendritic cells, macrophages M0, macrophages M1, macrophages M2, eosinophils, erythroblasts, basophils, and myeloid-derived suppressor cells (MDSCs). In other embodiments, cells for isolation of a pericellular proteome comprise brain cells, which may be selected from neuronal cells, astrocytes, oligodendrocytes, microglia, and progenitor cells thereof.

In some variations of a method for isolating a pericellular proteome, the cells are tumor (cancer) cells. In some such embodiments, the tumor cells are solid tumor cells. Exemplary solid tumor cells for use in accordance with the methods disclosed herein include cells from a cancer selected from a breast cancer (e.g., metastatic breast cancer or inflammatory breast cancer); a lung cancer (e.g., non-small cell lung cancer, small cell carcinoma, or mesothelioma); a cancer of the head and neck (e.g., a cancer of the oral cavity, orophyarynx, nasopharynx, hypopharynx, nasal cavity or paranasal sinuses, larynx, lip, or salivary gland); a gastrointestinal tract cancer (e.g., colorectal cancer, gastric cancer, esophageal cancer, or anal cancer); gastrointestinal stromal tumor (GIST); pancreatic adenocarcinoma; pancreatic acinar cell carcinoma; a cancer of the small intestine; a cancer of the liver or biliary tree (e.g., liver cell adenoma, hepatocellular carcinoma, hemangiosarcoma, extrahepatic or intrahepatic cholangiosarcoma, cancer of the ampulla of vater, or gallbladder cancer); a gynecologic cancer (e.g., cervical cancer, ovarian cancer, fallopian tube cancer, peritoneal carcinoma, vaginal cancer, vulvar cancer, gestational trophoblastic neoplasia, or uterine cancer, including endometrial cancer or uterine sarcoma); a cancer of the urinary tract (e.g., prostate cancer; bladder cancer; penile cancer; urethral cancer, or kidney cancer such as, for example, renal cell carcinoma or transitional cell carcinoma, including renal pelvis and ureter); testicular cancer; a cancer of the central nervous system (CNS) such as an intracranial tumor (e.g., astrocytoma, anaplastic astrocytoma, glioblastoma, oligodendroglioma, anaplastic oligodendroglioma, ependymoma, primary CNS lymphoma, medulloblastoma, germ cell tumor, pineal gland neoplasm, meningioma, pituitary tumor, tumor of the nerve sheath (e.g., schwannoma), chordoma, craniopharyngioma, a chloroid plexus tumor (e.g., chloroid plexus carcinoma), or other intracranial tumor of neuronal or glial origin) or a tumor of the spinal cord (e.g., schwannoma, meningioma); an endocrine neoplasm (e.g., thyroid cancer such as, for example, thyroid carcinoma, medullary cancer, or thyroid lymphoma; a pancreatic endocrine tumor such as, for example, an insulinoma or glucagonoma; an adrenal carcinoma such as, for example, pheochromocytoma; a carcinoid tumor; or a parathyroid carcinoma); a skin cancer (e.g., squamous cell carcinoma; basal cell carcinoma; Kaposi's sarcoma; or a malignant melanoma such as, for example, an intraocular melanoma); a bone cancer (e.g., a bone sarcoma such as, for example, osteosarcoma, osteochondroma, or Ewing's sarcoma); multiple myeloma; a chloroma; a soft tissue sarcoma (e.g., a fibrous tumor or fibrohistiocytic tumor); a tumor of the smooth muscle or skeletal muscle; a blood or lymph vessel perivascular tumor (e.g., Kaposi's sarcoma); a synovial tumor; a mesothelial tumor; a neural tumor; a paraganglionic tumor; an extraskeletal cartilaginous or osseous tumor; and a pluripotential mesenchymal tumor. In some embodiments, tumor cells for use in accordance with the methods disclosed herein includes cells from a cancer selected from breast cancer, small-cell lung cancer, non-small cell lung cancer, hepatocellular carcinoma, melanoma, renal cell carcinoma, basal cell carcinoma, cutaneous squamous cell carcinoma, esophageal cancer, pancreatic cancer, glioblastoma, medulloblastoma, gliosarcoma, cervical cancer, ovarian cancer, urothelial carcinoma, gastric carcinoma, colon cancer, colorectal cancer, testicular cancer, prostate cancer, thyroid cancer, and Merkel cell carcinoma.

In other variations of a method for isolating a pericellular proteome, the cells are non-solid (hematopoietic) tumor cells. Exemplary non-solid tumor cells for use in accordance with the methods disclosed herein include cells from a hematological malignancy selected from a non-Hodgkin lymphoma (e.g., B-cell lymphoma, T-cell lymphoma, or undifferentiated lymphoma), a leukemia (e.g., chronic myelogenous leukemia, hairy cell leukemia, chronic lymphocytic leukemia, chronic myelomonocytic leukemia, acute myelocytic leukemia, or acute lymphoblastic leukemia), a myeloproliferative disorder (e.g., multiple myeloma, essential thrombocythemia, myelofibrosis with myeloid metaplasia, hypereosinophilic syndrome, chronic eosinophilic leukemia, or polycythemia vera), and Hodgkin lymphoma.

In some embodiments of a method for isolating a pericellular proteome, the cells are solid tumor cells derived from an adult or pediatric carcinoma, sarcoma, melanoma, or brain-derived cancer that forms 3D masses. Such solid tumors include, for example, prostate cancer, breast cancer (e.g., estrogen receptor positive (ER+) breast cancer or triple negative breast cancer), small-cell lung cancer, non-small cell lung cancer, osteosarcoma, gastrointestinal stromal tumor, neuroblastoma, Ewing's sarcoma, rhabdomyosarcoma, angiosarcoma, hepatocellular carcinoma, melanoma, renal cell carcinoma, basal cell carcinoma, cutaneous squamous cell carcinoma, head and neck cancer, nasopharyngeal carcinoma, esophageal cancer, gastric carcinoma, colon cancer, colorectal cancer, biliary cancer, pancreatic cancer, glioblastoma, astrocytoma, medulloblastoma, retinoblastoma, thyroid carcinoma, cervical cancer, ovarian cancer, urothelial carcinoma, testicular cancer, germ cell tumor, and Merkel cell carcinoma.

In other embodiments, the cells are non-solid tumor cells that form 3D masses such as, for example, cells from a leukemia (myeloid sarcomas; e.g., acute myeloid leukemia), cells from a lymphoma (lymphoid tumors), or cells from a myeloma (extramedullar soft tissue myelomas).

In some variations, the plurality of cells are from a cell line, whereby the method comprises culturing the plurality of cells in vitro. Generally, any cell line that forms multicellular aggregates is particularly suitable for use in accordance with the disclosed method. Exemplary cell lines include MCF-7, MCF-7:WS8, HCC70, MDA-MB-468, HDQP1, CAL851, DMS53, NCI-H1048, LNCaP, and PC-3 cells.

In other variations, the plurality of cells are from a primary tissue (e.g., a primary tumor sample) harvested from an organism (e.g., from a human patient or from an animal model), whereby the method comprises culturing the plurality of cells ex vivo. In some particular ex vivo embodiments, the plurality of cells are from a patient-derived xenograft organoid model. For example, in specific variations, a patient-derived tumor is xenografted into an animal (e.g., a mouse), and the tumor that grows is harvested from the animal is used to generate organoids ex vivo. Those organoids are then subjected to the bioorthogonal labeling procedure in 3D culture as described herein.

In some embodiments, the plurality of cells comprises at least two different cell types. Such embodiments are particularly advantageous for investigating intercellular signaling in heterogeneous cell populations such as, for example, interactions between tumor cells and other, non-cancerous cells in the tumor microenvironments, including, e.g., fibroblasts, endothelial cells, pericytes, neurons, adipocytes, and adaptive and innate immune cells. In some variations comprising tumor cells, a heterogeneous population of cells include a non-cancerous cell selected from primary HMVEC endothelial cells, HUVEC endothelial cells, human visceral adipocytes, normal human lung fibroblasts (NHLF), neonatal cardiomocytes, and primary B and T cells separated by fluorescence-activated cell sorting. In certain embodiments, the at least two different cell types comprise a tumor cell and an immune cell such as, for example, a monocyte, a neutrophil, an eosinophil, a basophil, or a lymphocyte (e.g., a natural killer cell (NK cell), T-cell, or B-cell).

In some embodiments as above comprising at least two different cell types, the amino acid analog is a “mutant-specific” amino acid analog that is selectively incorporated by a mutant t-RNA synthetase and one of the at least two different cell types is a genetically engineered cell expressing the mutant t-RNA synthetase, whereby the mutant-specific amino acid analog is selectively incorporated into proteins synthesized by the genetically engineered cell but not into proteins synthesized by the other cell type(s) with the culture system. Such embodiments are especially advantageous for isolating and detecting pericellular proteins expressed by a particular cell type in a heterogeneous cell population. For example, in certain variations of the method comprising at least two different cell types and wherein the cell types include a tumor cell and an immune cell, either the tumor cell or the immune cell can be a genetically engineered cell (e.g., a genetically engineered cell line) expressing a mutant t-RNA synthetase that specifically incorporates the mutant-specific amino acid analog. Using such embodiments, the pericellular proteins expressed by either the immune cell or the tumor cell can be specifically interrogated to dissect more specifically the intercellular signaling pathways involved in the tumor-immune interactions, including, for example, mechanisms of immune evasion (e.g., downmodulation of tumor antigen presentation, changes within the tumor microenvironment, dysregulation of antigen presenting cells, induction of T cell tolerance, increased expression of co-inhibitory signals, or induction of regulatory T cells).

Mutant t-RNA synthetases and their cognate mutant-specific amino acid analogs that are suitable for cell-specific labeling in accordance with the disclosed methods are known. See, e.g., Mahdavi et al., J. Am. Chem. Soc. 138:4278-4281, 2016; Yang et al., J. Am. Chem. Soc. 23:7046-7051, 2018. For example, the mutant t-RNA synthetase L274GMmMetRS (full amino acid sequence shown in SEQ ID NO:1), which selectively incorporates the amino acid analog azidonorleucine, is obtained from a single mutation of the Mus Musculus methionine t-RNA synthetase at the L274 position, converting the Leucine (L) to glycine (G). See Mahdavi et al., supra. See also Link et al., Proc. Natl. Acad. Sci. USA 103:10180-10185, 2006 (describing identification of mutant versions of MetRS that incorporate azidonorleucine). Another example is the mutant tRNA-synthetase T413GMmPheRS (full amino acid sequence shown in SEQ ID NO:2), which selectively incorporates p-azido-L-phenylalanine, is obtained from a single point mutation of the Mus musculus phenylalanyl t-RNA synthetase at the T413 position, converting the Threonine residue (T) to glycine (G). See Yang et al., supra. In yet another example, the mutant tRNA-synthetase Y43GScTyrRS (full amino acid sequence shown in SEQ ID NO:3), which selectively incorporates the amino acid analog 3-azido-L-tyrosine, is obtained from a single point mutation of the Saccharomyces cerevisiae tyrosyl t-RNA synthetase at the Y43 position, converting the Tyrosine residue (Y) to Glycine (G). Any of the aforementioned mutant t-RNA synthetases, together with its cognate mutant-specific amino acid analog, may be used to selectively label pericellular proteins synthesized by genetically engineered cells expressing the mutant t-RNA synthetase (i.e., cells that have been transiently or stably transfected or transduced with a gene encoding the mutant t-RNA synthetase using, for example, transfection or transduction techniques well known in the art).

In some embodiments, the cells are cultured in the presence of a naturally occurring factor such as, for example, a cytokine, chemokine, growth factor, or hormone. Such embodiments are useful, e.g., for investigating pericellular proteins that may exhibit altered (e.g., increased) expression levels in response to such factors. For example, in some embodiments, the cells are tumor cells cultured in the presence of a cytokine, chemokine, growth factor, or hormone associated with tumorigenesis. In some such variations, the cells are ER+ breast cancer cells and the cells are cultured in the presence of estradiol. In other variations, breast cancer cells (e.g., triple-negative or ER+) are cultured in the presence of an EGF receptor (EGFR) family ligand, or a ligand of the EGFR interactome, such as, for example, amphiregulin (AREG), epidermal growth factor (EGF), urokinase-type plasminogen activator (uPA/PLAU), epigen (EPGN), epiregulin (EREG), transforming growth factor alpha (TGF-α), heparin-binding EGF-like growth factor (HB-EGF), or betacellulin. In other variations, the cells are small cell carcinoma cells cultivated in the presence of fibroblast growth factor (FGF) or estradiol. In other variations, the cells are prostate carcinoma cells cultivated in the presence of dihydrotestosterone.

In other embodiments, the cells are cultured in the presence of a drug. Such embodiments are useful, e.g., for investigating pericellular proteins that may exhibit altered (e.g., increased) expression levels in response to a drug and which may be involved, for example, in a therapeutic response to the drug and/or tolerance or resistance to the drug. For example, in some embodiments, the cells are tumor cells cultured in the presence of an anti-cancer drug such as, e.g., a hormonal therapeutic or chemotherapeutic drug. In some such variations, the tumor cells are breast cancer cells (e.g., ER+ breast cancer cells) and the cells are cultured in the presence of a hormonal therapeutic drug selected from tamoxifen and fulvestrant. In other variations, the cells are ER+ breast cancer cells cultured in the presence of a PI3K inhibitor (e.g., alpelisib), triple-negative breast cancer cells cultured in the presence of a small-molecule EGFR inhibitor (e.g., erlotinib, gefitinib); triple-negative breast cancer cells cultured in the presence of an EGFR-targeting monoclonal antibody therapeutic (e.g., cetuximab). In still other variations, the cells are small cell carcinoma cells cultured in the presence of platinum and etoposide, or an FGFR inhibitor (e.g., PD0173074, BGJ398). In yet other variations, the cells are prostate carcinoma cells cultured in the presence of androgen receptor antagonist enzalutamide.

### III. Methods of Isolating Intercellular Proteins In Vivo

In another aspect, the present disclosure provides a method for isolating proteins that are pericellularly localized in an animal in vivo. In some embodiments, the method generally includes the following steps: (a) implanting into an animal a plurality of cells, wherein the plurality of cells are genetically engineered to express a mutant t-RNA synthetase that selectively incorporates a mutant-specific amino acid analog; (b) administering the mutant-specific amino acid analog to the animal, wherein the mutant-specific amino acid analog is functionalized with a first chemical group that is reactive in a bioorthogonal chemical reaction, and whereby the mutant-specific amino acid analog is selectively incorporated into proteins synthesized by the implanted, genetically engineered cells but not into proteins synthesized by the other cells within the animal; (c) administering a cell-membrane-impermeable probe to the animal, wherein the probe has the formula A-L-D, wherein A is a molecule comprising a second chemical group that is reactive in a bioorthogonal chemical reaction, wherein the first chemical group is reactive with second chemical group, L is a hydrophilic linker, and D is a molecule comprising a biotin analog moiety capable of specifically binding to streptavidin with a dissociation constant greater than about 1 pM and an off-rate kinetic constant greater than about 1×10⁻³ l/s, and wherein proteins that are synthesized by the implanted cells and pericellularly localized are labeled with the probe by a bioorthogonal chemical reaction between the first and second chemical groups; (c) harvesting a tissue comprising the implanted cells from the animal; (d) lysing the harvested tissue to generate a lysate containing the labeled proteins and other cellular and extracellular components; (e) contacting the lysate with a capture reagent comprising a biotin-binding protein immobilized on a solid support, wherein the biotin-binding protein binds to the biotin analog moiety on the labeled proteins to yield a [labeled protein]:probe complex; and (f) separating the solid support from the lysate to separate the labeled proteins from the other cellular and extracellular components, thereby isolating the proteins that are pericellularly localized in the animal.

A variety of different cell types may be used for in vivo implantation and isolation of a cell-specific pericellular proteome in accordance with the above method. Suitable cell-types include those discussed above in the context of in vitro and ex vivo methods (see discussion of different cell types under section II, supra). Particularly suitable cells for use in accordance with the present in vivo methods are tumor cells. In some such embodiments, the tumor cells are solid tumor cells such as, e.g., cells derived from an adult or pediatric carcinoma, sarcoma, melanoma, or brain-derived cancer that forms 3D masses. Exemplary solid tumor cells are cells derived from a cancer selected from prostate cancer, breast cancer (e.g., estrogen receptor positive (ER+) breast cancer or triple negative breast cancer), small-cell lung cancer, non-small cell lung cancer, osteosarcoma, gastrointestinal stromal tumor, neuroblastoma, Ewing's sarcoma, rhabdomyosarcoma, angiosarcoma, hepatocellular carcinoma, melanoma, renal cell carcinoma, basal cell carcinoma, cutaneous squamous cell carcinoma, head and neck cancer, nasopharyngeal carcinoma, esophageal cancer, gastric carcinoma, colon cancer, colorectal cancer, biliary cancer, pancreatic cancer, glioblastoma, astrocytoma, medulloblastoma, retinoblastoma, thyroid carcinoma, cervical cancer, ovarian cancer, urothelial carcinoma, testicular cancer, germ cell tumor, and Merkel cell carcinoma. In other embodiments, the cells are non-solid tumor cells that form 3D masses such as, for example, cells from a leukemia (myeloid sarcomas; e.g., acute myeloid leukemia), cells from a lymphoma (lymphoid tumors), or cells from a myeloma (extramedullar soft tissue myelomas).

Typically, the genetically engineered cells are cell lines that have been stably transfected or transduced with a gene encoding the appropriate mutant t-RNA synthetase using well-known techniques for DNA transfection or viral transduction (e.g., lentiviral transduction) and selection of cells (which may include isolation and expansion of select clones) containing the transgene integrated into the cellular genome. Suitable techniques for selection of cells containing a transgene are well known and include, e.g., the use of vectors comprising a selective marker conferring antibiotic resistance and growth of cells in the presence of the antibiotic.

In the case of tumor cells, for example, xenograft models utilizing established cell lines or patient-derived cell lines are generally well-known and can be readily adapted for use in accordance with the present in vivo methods. See, e.g., Huo et al., Transl. Lung Cancer Res. 9:2214-2232, 2020 (xenograft models of lung cancer); Georges et al., Front. Med. 6:139, 2019 (xenograft models of colorectal cancer); Souto et al., J. Mammary Gland Biol. Neoplasia 27:211-230, 2022 (xenograft models of breast cancer); Haddad et al., Neuro-Oncol. Adv. 3:1-16, 2021 (xenograft models of glioblastoma). Exemplary xenograft models of breast cancer are also described in Wrenn et al. Cell 183:395-410, 2020; and Cocce et al., Cell Reports 29:889-903, 2019. An exemplary xenograft model of prostate cancer is described in Nguyen et al. The Prostate 77:6:654-671.2017.

Suitable animals for implantation of cells include, for example, mammals (e.g., mice, rats, other rodents, rabbits, dogs, cats, or pigs), birds, and fish (e.g., zebrafish). In some embodiments, an animal for implantation of cells is at an embryonic state of development (e.g., chick embryos, including the chicken egg chorioallantoic membrane (CAM)).

Implantation of cells may be carried out, e.g., by a surgical procedure under general anesthesia and analgesia. For example, following a small surgical incision at an appropriate location, cells may be transplanted (e.g., by injection of a cell suspension) at the desired site (e.g., under the skin or into an organ or tissue type from which the cells originated). An organ or tissue may be gently exteriorized as needed for implantation and then returned (e.g., in some models of colorectal cancer, the caecum is exteriorized before injection of tumor cells into the caecal wall before returning the caecum to the abdominal cavity). Following implantation of cells at the desired site, the incision is closed.

For implantation of human cells, an immunocompromised animal is typically used. Particularly suitable immunocompromised animals include immunocompromised mice such as, for example, athymic nude mice, severely compromised immunodeficient (scid) mice, or other immunocompromised mice. See, e.g., Morton and Houghton, Nat. Protoc. 2:247-250, 2007. A particularly suitable immunocompromised mouse strain is the NOD-scid strain, generated by backcrossing of scid mice to the non-obese diabetic (NOD) strain, which does not express the H-2g7 major histocompatability complex (MHC) haplotye and possesses a CLTA-4 alteration causing diabetes-induced autoimmunity. The NOD-scid strain lacks T and B cells and has lower NK-cell activity, allowing a higher rate of engraftment of human hematopoietic stem cells, leukemias, as well as breast and other cancers. Other suitable immunocompromised animal hosts include, e.g., immunocompromised rat and pig, zebrafish, and the chicken egg chorioallantoic membrane (CAM).

In some variations, a humanized animal model (e.g., a humanized mouse model) may be used. For example, humanized mouse models have been developed to examine interactions between immune components and human tumors. Such models involved engraftment of both human tumor and immune cells. Peripheral blood mononuclear cells (PBMCs) and human CD34+ hematopoietic stem cells have been successfully engrafted in immunodeficient mice to establish a functional immune system and develop models such as Hu-PBL (peripheral blood lymphocytes), Hu-CD34+, and BLT (bone marrow-liver-thymus) mice. See, e.g., Mosier et al., Nature 335:256-2599, 1988; Shultz et al., J. Immunol. 174:6477-6489, 2005; Traggiai et al., Science 304:104-107, 2004; Holyoake et al., Exp. Hematol. 27:1418-1427, 1999; Lan et al., Blood 108:487-492, 2006.

Following implantation of cells into the animal, the mutant specific amino acid analog is administered to the animal. The amino acid analog is administered at a dose and for a time sufficient to allow the amino acid analog to be assimilated by the implanted cells and incorporated into protein synthetic pathways, whereby the amino acid analog is incorporated into proteins synthesized by the implanted, genetically engineered cells expressing the mutant t-RNA synthetase. The amino acid analog may be delivered, e.g., at doses ranging from about 10 mg/kg to about 100 mg/kg for a duration of 1 to 10 days. For example, the dose may be delivered daily in the form of a saline solution by intraperitoneal injection, or daily by supplementation in the animal's drinking water in combination with maltose at doses ranging from 1 to 60 mM.

As in the in vitro and ex vivo methods described above, the amino acid analog is functionalized with a first chemical group that is reactive in a biorthogonal chemical reaction with a second chemical group that is present in the cell-membrane impermeable probe. Suitable complementary pairs of bioorthogonal chemical groups include those discussed above in the context of in vitro and ex vivo methods (see discussion of first and second chemical groups under section II, supra). A particularly suitable complementary pair of bioorthogonal chemical groups for use in accordance with the present methods is a strained alkyne and an azide. Accordingly in some variations, the functionalized amino acid analog comprises a first chemical group that is an azide. Particularly suitable mutant-specific azide-containing amino acid analogs for use in accordance with the present disclosure include azidonorleucine, p-azido-Lphenylalanine, and 3-azido-L-tyrosine (see Table 1, supra).

Mutant t-RNA synthetases suitable for use with the present in vivo methods include those discussed above in the context of certain in vitro and ex vivo embodiments (see discussion of mutant t-RNA synthetases under section II, supra). In some embodiments, the mutant t-RNA synthetase is L274GMmMetRS (full amino acid sequence shown in SEQ ID NO:1) and the mutant-specific amino acid analog is azidonorleucine. In other embodiments, the mutant tRNA-synthetase is T413GMmPheRS (full amino acid sequence shown in SEQ ID NO:2) and the mutant-specific analog is p-azido-L-phenylalanine. In yet other embodiments, the mutant tRNA-synthetase is Y43GScTyrRS (full amino acid sequence shown in SEQ ID NO:3) and the mutant-specific amino acid analog is 3-azido-L-tyrosine.

Following incorporation of the mutant-specific amino acid analog into newly synthesized proteins, the cell-membrane-impermeable probe is administered to the animal. Suitable probes include those discussed above in the context of in vitro and ex vivo methods (see discussion of probes under section II, supra, including variations of molecules comprising the second chemical group (A), hydrophilic linkers (L), and biotin analog moieties (D)). The probe is administered at a dose and for a time sufficient to allow the probe to infiltrate intercellular spaces and label pericellular proteins synthesized by the implanted cells via the bioorthogonal chemical reaction between the incorporated amino acid analog comprising the first chemical group (e.g., an azide) and the second chemical group (e.g., a strained alkyne) of the probe, thereby functionalizing the pericellular proteins with the biotin analog moiety of the probe. In some variations, the probe is administered as a saline solution containing the probe at a concentration of from about 0.01 mM to about 0.3 mM; a volume of about 50 to 200 μL of such a solution may be delivered by one-time intravenous injection (for example, in the tail vein of mice), intraperitoneal injection, or intratumoral injection. Typically, the probe is allowed to circulate and undergo the bioorthogonal reaction for a period of about 30 minutes to 4 hours, after which point the animal is euthanized and the tissue is harvested.

In some embodiments, the animal is treated with a naturally occurring factor such as, for example, a cytokine, chemokine, growth factor, or hormone before labeling pericellular proteins with the probe. Such embodiments are useful, e.g., for investigating pericellular proteins that may exhibit altered (e.g., increased) expression levels in vivo in response to such factors. For example, in some embodiments, the implanted cells are tumor cells and a cytokine, chemokine, growth factor, or hormone associated with tumorigenesis is administered to the animal before administering the probe. In some such variations, the implanted cells are ER+ breast cancer cells and estradiol is administered to the animal. In other variations, breast cancer cells (e.g., triple-negative or ER+) are cultured in the presence of an EGF receptor (EGFR) family ligand, or a ligand of the EGFR interactome, such as, for example, amphiregulin (AREG), epidermal growth factor (EGF), urokinase-type plasminogen activator (uPA/PLAU), epigen (EPGN), epiregulin (EREG), transforming growth factor alpha (TGF-α), heparin-binding EGF-like growth factor (HB-EGF), or betacellulin. In other variations, the cells are small cell carcinoma cells cultivated in the presence of fibroblast growth factor (FGF) or estradiol. In other variations, the cells are prostate carcinoma cells cultivated in the presence of dihydrotestosterone.

In other embodiments, the animal is treated with a drug before labeling pericellular proteins with the probe. Such embodiments are useful, e.g., for investigating pericellular proteins that may exhibit altered (e.g., increased) expression levels in vivo in response to a drug and which may be involved, for example, in a therapeutic response to the drug and/or tolerance or resistance to the drug. Drugs for administration may include, for example, small-molecule drugs as well as biologics (e.g., monoclonal therapeutic antibodies and antibody-drug conjugates). For example, in some embodiments, the implanted cells are tumor cells and an anti-cancer drug such as, e.g., a hormonal therapeutic or chemotherapeutic drug, or a monoclonal therapeutic antibody or antibody-drug conjugate, is administered to the animal before administering the probe. In some such variations, the tumor cells are breast cancer cells (e.g., ER+ breast cancer cells) and a hormonal therapeutic drug selected from tamoxifen and fulvestrant is administered to the animal. In other variations, the cells are breast cancer cells (e.g., triple-negative breast cancer cells) and an EGFR-targeting drug or therapeutic antibody is administered to the animal (e.g., small-molecule EGFR inhibitors such as erlotinib or gefitinib; monoclonal EGFR-targeting antibodies such as cetuximab). In still other variations, the cells are ER+ breast cancer cells and a PI3K kinase inhibitor (e.g., alpelisib) is administered to the animal.

Following labeling of cells in vivo with the cell-membrane-impermeable probe, a tissue comprising the implanted cells (e.g., an organ comprising the implanted cells, or a more targeted section of tissue primarily consisting of the implanted cells) is harvested (e.g., surgically removed) from the animal. Methods and materials (e.g., instruments) to isolate tissues and organs from animal subjects are generally known in the art. Any appropriate method to isolate organs or tissues as described herein can be used.

Once harvested, the harvested tissue is subjected to lysis to generate a lysate containing the labeled proteins and other cellular and extracellular components. As discussed above in the context of in vitro and ex vivo methods, methods for lysing cells for subsequent biomolecular analysis are generally well-known in the art (see discussion of lysis methods under section II, supra), and such methods may also be used to lyse tissues harvested from an animal in accordance with the present in vivo methods.

Following lysis, labeled pericellular proteins are isolated and optionally analyzed as described above in the context of in vitro and ex vivo methods. See discussion of protein isolation using immobilized biotin-binding protein, elution with free biotin or analogs thereof, and downstream analysis of proteins, including protein identification and quantitation, under section II, supra.

### IV. Methods of Isolating Lipidated Intercellular Proteins In Vitro or Ex Vivo

In another aspect, the present disclosure provides a method for isolating lipidated proteins that are pericellularly localized in vitro or ex vivo. In some embodiments, the method generally includes the following steps: (a) culturing a plurality of cells in a complex cell culture system, wherein the plurality of cells are cultured in the presence of an alkanoic acid analog functionalized with a first chemical group that is reactive in a bioorthogonal chemical reaction, and wherein the alkanoic acid analog is incorporated into newly synthesized lipidated proteins; (b) contacting the culture system with a cell-membrane-impermeable probe having the formula A-L-D, wherein A is a molecule comprising a second chemical group that is reactive in a bioorthogonal chemical reaction, wherein the first chemical group is reactive with second chemical group, L is a hydrophilic linker, and D is a molecule comprising a biotin analog moiety capable of specifically binding to streptavidin with a dissociation constant greater than about 1 pM and an off-rate kinetic constant greater than about 1×10⁻³ l/s, and wherein newly synthesized lipidated proteins that are pericellularly localized are labeled with the probe by a bioorthogonal chemical reaction between the first and second chemical groups; (c) lysing the cultured cells to generate a lysate containing the labeled proteins and other cellular and extracellular components; (d) contacting the lysate with a capture reagent comprising a biotin-binding protein immobilized on a solid support, wherein the biotin-binding protein binds to the biotin analog moiety on the labeled proteins to yield a [labeled protein]:probe complex; and (e) separating the solid support from the lysate to separate the labeled proteins from the other cellular and extracellular components, thereby isolating the lipidated proteins that are pericellularly localized in the multicellular system.

In some embodiments of a method as above, the alkanoic acid analog is a myristic acid analog or a palmitic acid analog.

Suitable first and second chemical groups that are reactive in a bioorthogonal chemical reaction include those discussed above in the context of in vitro and ex vivo methods utilizing functionalized amino acid analogs (see discussion of first and second chemical groups under section II, supra). A particularly suitable complementary pair of bioorthogonal chemical groups for use in accordance with the present methods is a strained alkyne and an azide. Accordingly in some variations, the functionalized alkanoic acid analog comprises a first chemical group that is an azide. Particularly suitable azide-containing alkanoic acid analogs for use in accordance with the present method for isolating lipidated proteins include 12-azidododecanoic acid or 15-azidopentadecanoic acid.

Suitable complex culture systems (e.g., 3D culture systems) and cell types include those discussed above in the context of in vitro and ex vivo methods utilizing functionalized amino acid analogs (see discussion of culture systems and cells under section II, supra). The functionalized alkanoic acid analog is added to the culture system media in a sufficient quantity to allow incorporation of the alkanoic acid analog into newly synthesized proteins. Concentrations of the functionalized alkanoic acid analog in the culture media typically range from about 0.01 mM to about 2 mM. More typically, the alkanoic acid analog is added to the culture media at a concentration of from about 0.01 mM to about 0.20 mM or from about 0.01 mM to about 0.06 mM. The alkanoic acid may be dissolved in a concentrated stock (e.g., 1,000 times concentrated) with a water-miscible organic solvent (e.g., DMSO) and diluted directly into cell culture media; there is no requirement for using alkanoic-acid depleted media. After sufficient time, generally from about 30 minutes to about 24 hours or more, the alkanoic acid analog is assimilated by the cells and incorporated into protein synthetic pathways, whereby the alkanoic acid analog is incorporated into newly synthesized lipidated proteins. Cells may also be cultured in the presence of other factors as described above (for example, in the presence of a naturally occurring factor such as, e.g., a cytokine, chemokine, growth factor, or hormone, or in the presence of a drug such as, e.g., an anti-cancer drug; see corresponding discussion under section II, supra).

Following incorporation of the alkanoic acid analog into newly synthesized lipidated proteins, newly synthesized lipidated proteins that are pericellulary localized are labeled with the cell-membrane-impermeable probe having the formula A-L-D in a bioorthogonal chemical reaction between the first bioorthogonal chemical group of the amino acid analog and the second bioorthogonal chemical group of probe, thereby functionalizing the pericellular lipidated proteins with the biotin analog moiety of the probe. Suitable probes and corresponding methods for labeling functionalized pericellular proteins are discussed above in the context of in vitro and ex vivo methods utilizing functionalized amino acid analogs (see discussion of probes and labeling methods under section II, supra, including variations of molecules comprising the second chemical group (A), hydrophilic linkers (L), and biotin analog moieties (D) and suitable probe concentrations and incubation periods).

Once functionalized lipidated proteins are labeled with the cell-membrane-impermeable probe, cells are lysed and labeled proteins are isolated, followed by any downstream analysis (e.g., detection, quantitation) as described above in the context of in vitro and ex vivo methods utilizing functionalized amino acid analogs (see discussion of cell lysis, isolation using immobilized biotin-binding protein and elution, and downstream analysis, including protein detection and quantitation, under section II, supra).

The invention is further illustrated by the following non-limiting examples.

### Example 1

DBCO-PEG4-Desthiobiotin enables specific labeling of the intercellular proteome in 3D culture. In certain variations, the disclosed method of pericellular protein isolation utilizes a probe with the following features. First, the probe reacts with proteins that are actively produced by cells in culture, and not react with any of the functional groups found in exogenous extracellular matrices, such as Engelbreth-Holm-Swarm (EHS) mouse sarcoma matrix proteins. Second, the probe does not pass through the cell membrane, so that labeling reactions occur only in the pericellular space, and not in the cytosol, nucleus, or other intracellular compartments. Third, the reaction is compatible with live-cell labeling, since conventional fixation approaches typically fail to retain soluble factors that accumulate in intercellular spaces and compromise the integrity of the cell membrane, rendering it permeable to small molecules. Fourth, upon reaction, the probe covalently attaches a moiety that enables downstream enrichment and recovery of labeled proteins. Fifth, the downstream enrichment approach enables elution of full-length proteins that are compatible with a broad range of downstream proteomic detection and quantification approaches, e.g., liquid-chromatography mass-spectrometry (LC/MS) and immunoaffinity-based detection methods.

A widely used chemistry for cell surface labeling in conventional 2D culture is the reaction between N Hydroxysuccinimide (NHS) and primary amines (such as those contained within lysine residues of proteins). A common approach involves using a sulfo-NHS-biotin reagent (or a similar cell-impermeant reagent containing an NHS moiety and a biotin moiety) to label all molecules on the cell surface and extracellular space, followed by immobilization on a streptavidin-based stationary phase. However, primary amines are abundantly present on all proteins, including EHS matrix. The NHS moiety also reacts with other nucleophiles common in biopolymers, such as hydroxyl groups, which are prominent in other 3D culture systems, such as agarose, alginate, hyaluronic acid, and dextran hydrogels. As such, this conventional method produces abundant off-target labeling in 3D culture, fails to label the intercellular proteome in 3D culture.

In contrast to the reaction of NHS esters with amines, the bioorthogonal cycloaddition click reactions between azides and alkynes exhibit low cross-reactivity with the functional groups that are typically found on biomolecules, e.g., amines, hydroxyl, carbonyl, carboxyl, thiol, ester, and ether groups. The azide-containing methionine analog, azidohomoalanine, can be recognized by the endogenous methionine t-RNA synthetase in mammalian cells, and incorporated into newly synthesized proteins. Azides can undergo cycloaddition reactions with terminal alkynes and strained alkynes (such as DBCO). However, the reaction with terminal alkynes requires a copper catalyst, which is highly cytotoxic and unsuitable for live-cell labeling. In contrast, strained alkynes (such as DBCO and BCN) can undergo a strain-promoted azide alkyne cycloaddition (SPAAC) reaction in the absence of a copper catalyst. Thus, by incorporating either an azide or an alkyne into newly synthesized proteins, the SPAAC reaction could be used to selectively label only cell-surface proteins, which would help discriminate between proteins produced in the pericellular space from those expressed in the exogenous extracellular matrix. The presently disclosed method focuses on performing a bioorthogonal reaction in 3D culture that specifically labels the pericellular proteins, and thereby enabling recovery of full-length proteins for enrichment and proteomic analysis.

Such an approach has multiple structural considerations. First, due to the phospholipid-bilayer structure of the cell membrane, highly hydrophobic small molecules are more cell permeable than those that are hydrophilic or negatively charged. The strained alkynes that participate in SPAAC reactions with azides (including BCN and DBCO) are highly hydrophobic. Thus, to limit the SPAAC reactions to only the pericellular spaces of living cells, the membrane-impermeable probes would require hydrophilic linkers between the click reaction partner and the enrichment moiety, e.g., negatively charged linkers containing sulfonated group, or uncharged polyethylene glycol (PEG). Second, it was important to utilize an enrichment moiety that would enable immobilization on a stationary phase and subsequent elution of full-length proteins under mild conditions, thereby yielding proteins that would be amenable to a broad range of downstream analytical approaches, including immunoaffinity-based detection methods (e.g., western blotting, ELISA, the proximity extension assay (PEA)). The biotin-streptavidin interaction is widely used for enrichment of biomolecules in proteomics and other applications. However, the biotin-streptavidin interaction is the strongest non-covalent bond known in nature. It is known to be irreversible under physiological conditions, requiring harsh denaturing conditions that produce poor recoveries and involve denaturants that are incompatible with immunoaffinity detection. Consequently, a common method for proteomics analysis following immobilization of biotinylated molecules with streptavidin is to perform an on-resin or on-bead digestion with proteases, which produces peptides suitable for LC/MS, but not immunoaffinity-based detection. Using a biotin analog with a weaker affinity for streptavidin, such as desthiobiotin, would, however, enable efficient enrichment of pericellular proteins that undergo the click reaction and release of full-length proteins under mild conditions.

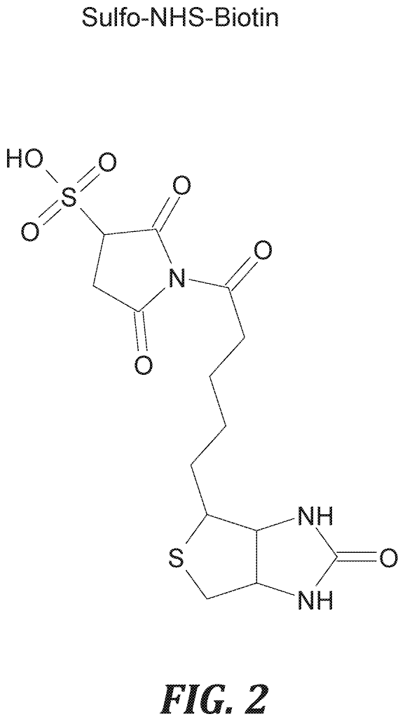

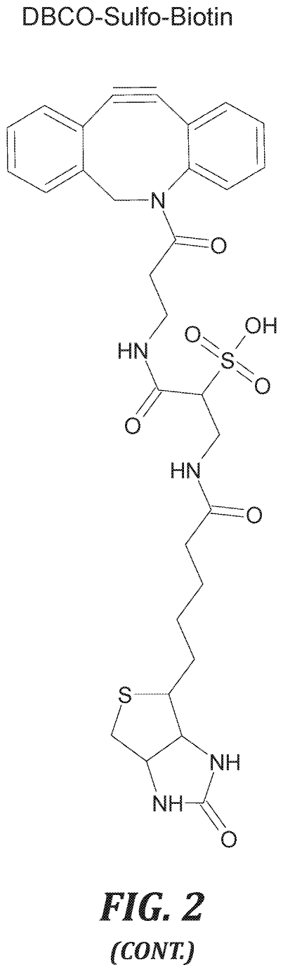

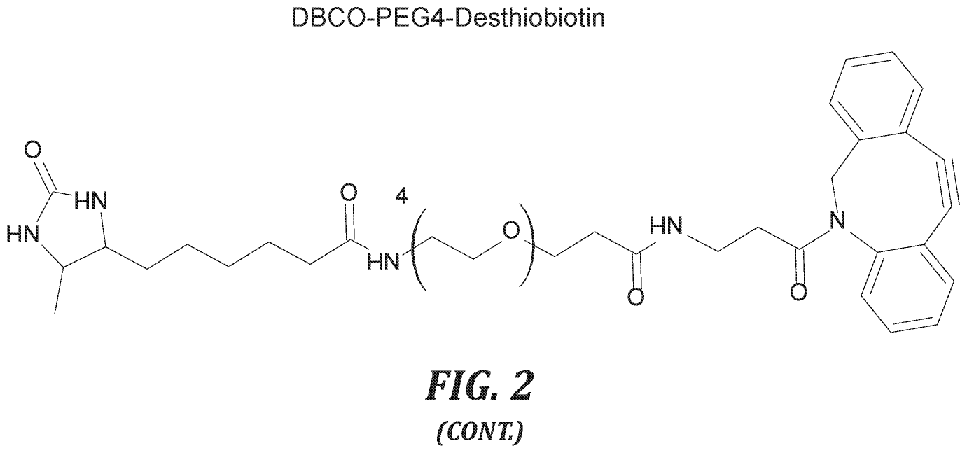

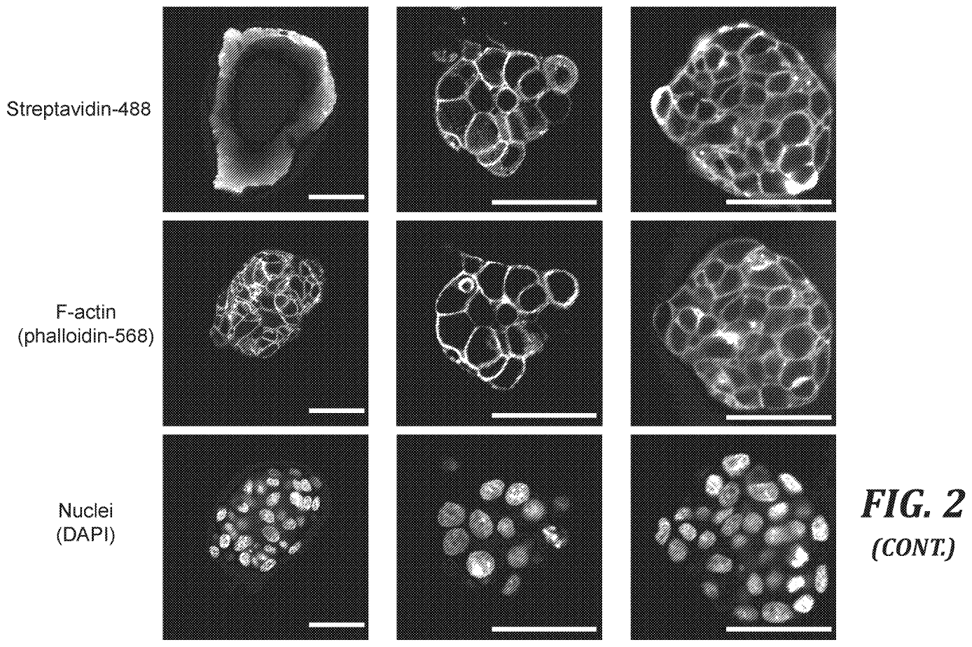

Considering these structural considerations, a probe consisting of DBCO moiety, a hydrophilic PEG linker, and a desthiobiotin enrichment moiety (DBCO-PEG4-Desthiobiotin, FIG. 2) would enable specific labeling of pericellular proteins in 3D culture in the presence of an exogenous EHS matrix, immobilization of labeled proteins on a streptavidin stationary phase, and elution of full-length proteins under mild conditions. To evaluate the performance of this probe in comparison to the widely used method of NHS labeling, the probe was directly compared to sulfo-NHS-biotin (FIG. 2). In addition, to specifically evaluate the advantage of using the desthiobiotin moiety, DBCO-PEG4-Desthiobiotin was compared to a reagent consisting of a DBCO click partner, a hydrophilic, sulfonated linker, and a biotin moiety (DBCO-sulfo-Biotin, FIG. 2).

Using confocal fluorescence microscopy, sulfo-NHS-biotin, DBCO-sulfo-Biotin, and DBCO-PEG4-Desthiobiotin were each evaluated for their ability to label the pericellular zone in 3D culture of triple-negative breast cancer cells in the presence of 2% EHS extracellular matrix. To do this, 3D organotypic suspension cultures of the HCC70 breast cancer cell line were generated in the presence of EHS matrix. Next, spheroids were labeled with 500 pM azidohomoalanine for 24 hours, and after washing excess reagent, incubated the spheroids with each of the heterobifunctional probes (three hundred micromolar concentration, 30 minutes at 37 degrees Celsius). Samples were then fixed and labeled with streptavidin conjugated to Alexa-Fluor 488 to identify the spatial distribution of either biotin or desthiobiotin. The samples were also counterstained with DAPI and Phalloidin to visualize the nuclei and the cell surface (F-actin in the HCC70 cells is subcortically localized near the membrane). Lastly, confocal fluorescence microscopy was used to qualitatively assess the spatial distribution of streptavidin labeling in all conditions (FIG. 2). The sulfo-NHS-biotin probe was unable to diffuse into intercellular zones of the organoids. Instead, the sulfo-NHS-biotin probe abundantly labeled a corona surrounding the exterior of the organoids that extended several microns beyond the cell boundary, which was presumed to be the exogenous EHS matrix. This is due to the propensity of the NHS group to rapidly and promiscuously react with primary amines and other nucleophiles that are abundantly present in the extracellular matrix, which in turn creates a reaction diffusion constraint that limits its ability to diffuse beyond the extracellular matrix and into the intercellular space. In contrast, both DBCO-containing reagents exhibited extensive labeling of the cells within the organoids. The DBCO-sulfo-biotin reagent and DBCO-PEG4-Desthiobiotin exhibited pericellular labeling, as indicated by co-localization with the cortical F-actin, with minimal evidence of cytoplasmic staining or labeling of the EHS matrix.

The next aim was to further characterize the specificity of the SPAAC reaction with the DBCO-PEG4-Desthiobiotin reagent in organotypic culture. To evaluate the extent to which the streptavidin signal produced was due to reactions with azide-labeled proteins, HCC70 breast cancer cell spheroids were cultured in 2% EHS matrix suspension culture with or without azidohomoalanine. We then incubated the spheroids were then incubated with DBCO-PEG4-Desthiobiotin, fixed half of the sample with 4% paraformaldehyde, 0.1% glutaraldehyde, and used the remaining sample to generate cell lysates for homogeneous streptavidin detection.

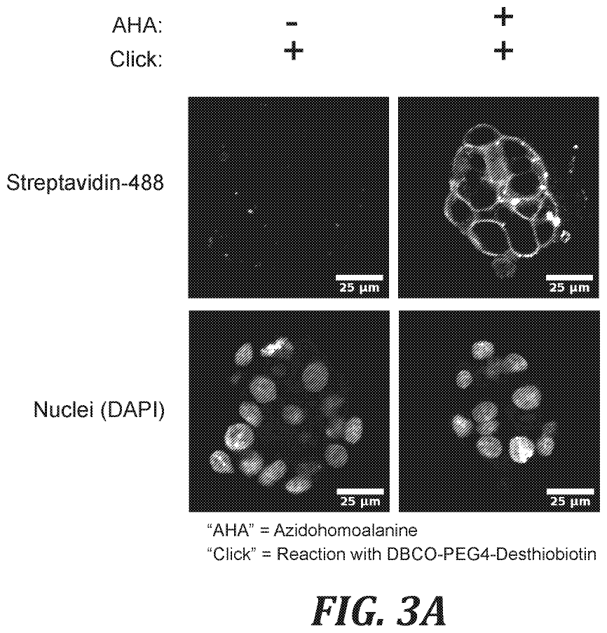

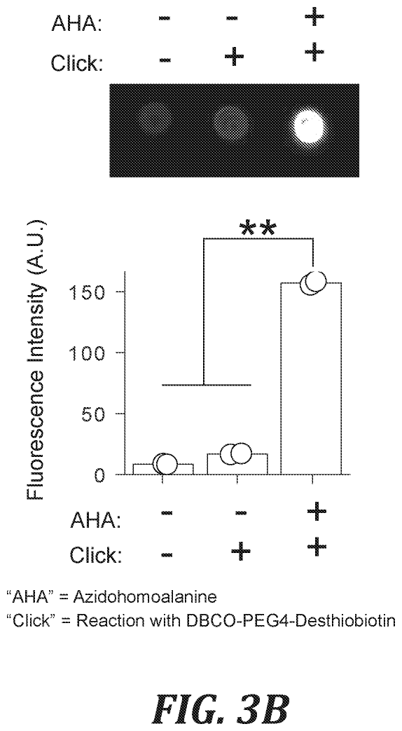

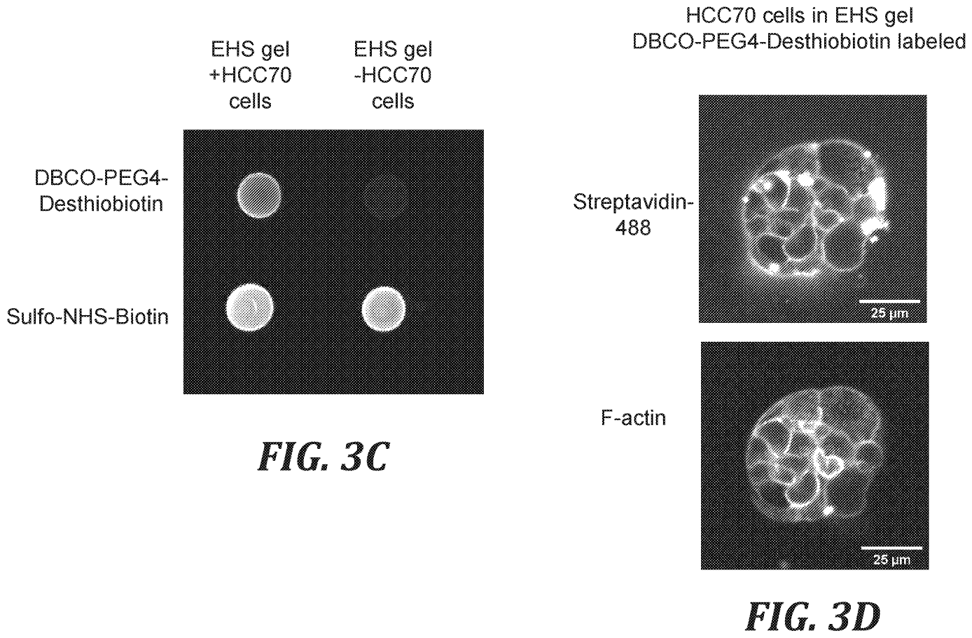

After labeling with streptavidin-alexa fluor 488, confocal fluorescence microscopy demonstrated substantial fluorescence only in azidohomoalanine-labeled spheroids (FIG. 3A), with minimal labeling in spheroids in media lacking azidohomolanine. To assess the specificity of the labeling, streptavidin dot blots were performed with homogeneous lysates prepared from each of the labeling conditions. Additionally, unlabeled lysate was included to account for non-specific binding of the streptavidin reagent, itself. Samples labeled with azidohomoalanine had 10-fold higher streptavidin binding compared to non-labeled azidohomoalanine controls (FIG. 3B). Together, these results indicate that most reactions undergone by the DBCO-PEG4-Desthiobiotin reagent were due to reactions with azide-labeled proteins and not due to reactions with the exogenous EHS matrix. Moreover, by bypassing the extracellular matrix, DBCO-containing reagents can diffuse into the intercellular spaces in 3D culture systems where NHS reagents fail. In the above experiments, the 2% EHS matrix suspension produces a culture system that behaved as a liquid on the macroscopic scale, but surrounding the cell spheroids, a micron-scale corona of denser extracellular matrix is organized, meaning the system exists at a concentration below the sol-gel percolation threshold. However, a common alternative organoid culture protocol involves fully embedding the cells in a higher concentration EHS matrix that forms a percolating elastic network hydrogel (above the sol-gel percolation transition). To further evaluate the ability of the DBCO-PEG4-Desthiobiotin reagent to bypass EHS matrix proteins and specifically react with azidohomoalanine-labeled proteins, the DBCO-PEG4-Desthiobiotin reagent was compared to sulfo-NHS-biotin in fully embedded 3D organotypic culture, wherein spheroids were cultured in a higher concentration EHS matrix (7-10 mg/mL protein, undiluted from the manufacturer's preparation), that forms an elastic hydrogel. Cultured HCC70 breast cancer spheroids were (1) embedded within an EHS matrix hydrogel, (2) labeled with azidohomoalanine, and (3) incubated with DBCO-PEG4-Desthiobiotin. As a non-specific reaction control, the same labeling conditions were evaluated in “empty” EHS matrix hydrogels, which contained no cells. Additionally, labeling with sulfo-NHS-biotin was performed in the same hydrogel conditions (with or without HCC70 cells embedded). After labeling and washing the samples, homogeneous lysates of all conditions were prepared.

The extent of biotin or desthiobiotin labeling was evaluated in all the above conditions using a streptavidin dot blot of the homogeneous lysates (FIG. 3C). In the case of sulfo-NHS-biotin, the fluorescent streptavidin binding signal was equal between the hydrogels that contained HCC70 spheroids and those that contained no cells, indicating that the labeling that occurred was due to off-target labeling of the EHS matrix. In contrast, in samples containing HCC70 cells, the DBCO-PEG4-Desthiobiotin reagent had a 10-fold higher streptavidin binding signal compared to the cell-free EHS matrix. DBCO-PEG4-Desthiobiotin labeled HCC70 cells embedded in EHS matrix gel were fixed and labeled with streptavidin-AF488 to confirm that DBCO-PEG4-Desthiobiotin labeled intercellular proteins in fully embedded 3D culture. The streptavidin signal predominantly localized to intercellular spaces colocalizing with the cortical F-actin (FIG. 3D). These experiments demonstrate that the DBCO-PEG4-Desthiobiotin reagent primarily bypasses the complex exogeneous matrix, even in high concentration elastic EHS matrix hydrogels, and labels azido pericellular proteins in applications where the widely used sulfo-NHS-biotin reagent fails.

The Desthiobiotin detection handle enables efficient capture and release offull-length pericellular proteins. Having established that a DBCO-containing click reaction probe can bypass EHS matrix and label pericellular proteins in 3D culture, the next aim was to evaluate the ability of these reagents to be used for enrichment and recovery of full-length labeled proteins. A key consideration for obtaining full-length proteins is the fact that the specific interaction through which the enrichment handle is detected allows reversible release after affinity-based capture. The biotin-streptavidin interaction is the strongest non-covalent interaction known in nature. Previous studies have shown that while dissociation of the biotin-streptavidin interaction can be achieved, it typically requires highly stringent denaturing conditions with urea, SDS, and boiling temperatures. Such protocols often provide poor recovery and are likely to interfere with direct applications in immunoaffinity-based detection, which requires the antibody to be in its native (non-denatured) form. Accordingly, the aim was to develop a heterobifunctional reagent containing a biotin analog (e.g., desthiobiotin, as used in this example) that would provide superior recovery of full-length proteins in comparison to a reagent containing the biotin enrichment handle, so that recovery could be achieved under more mild conditions that were suitable for a broader range of downstream applications.

To evaluate the recovery efficiency of DBCO-sulfo-biotin and DBCO-PEG4-desthiobiotin labeled spheroid samples, azidohomolanine-treated spheroids of HCC70 cells were prepared in 2% EHS matrix suspension. The pericelluar proteomes were then labeled using either DBCO-sulfo-biotin or DBCO-PEG4-desthiobiotin. After lysing the spheroids, biotinylated or desthiobiotinylated proteins were captured using streptavidin-functionalized magnetic polystyrene beads. The unbound fraction was also collected to evaluate the capture efficiency. In the case of the DBCO-sulfo-biotin samples, a stringent elution under denaturing conditions was performed with buffer containing 8M urea, 2% sodium dodecyl sulfate, and 3 mg/mL biotin, while heating at 100° C. for 1 hour, a protocol that was based on previous reports of the requirement for strong denaturing conditions for dissociation of the biotin-streptavidin bond. In the case of the DBCO-PEG4-desthiobiotin reagent, a milder elution was performed based on competitive binding with free biotin under non-denaturing conditions with 1% tritonx-100, 0.1% SDS, and 3 mg/mL biotin. This competitive elution was performed in two sequential incubations at 37° C. and 4° C.

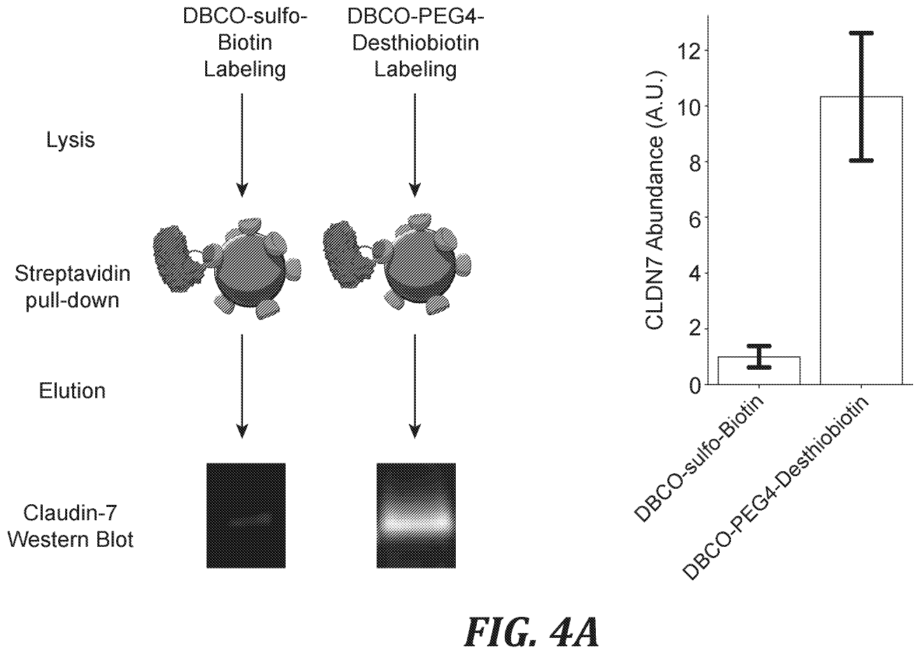

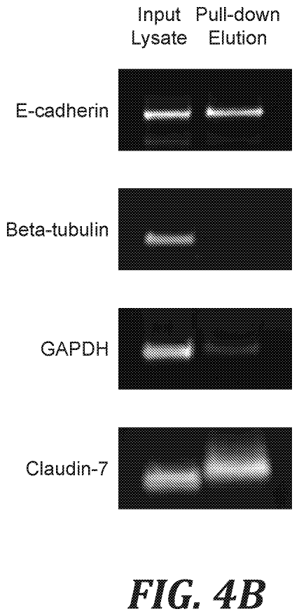

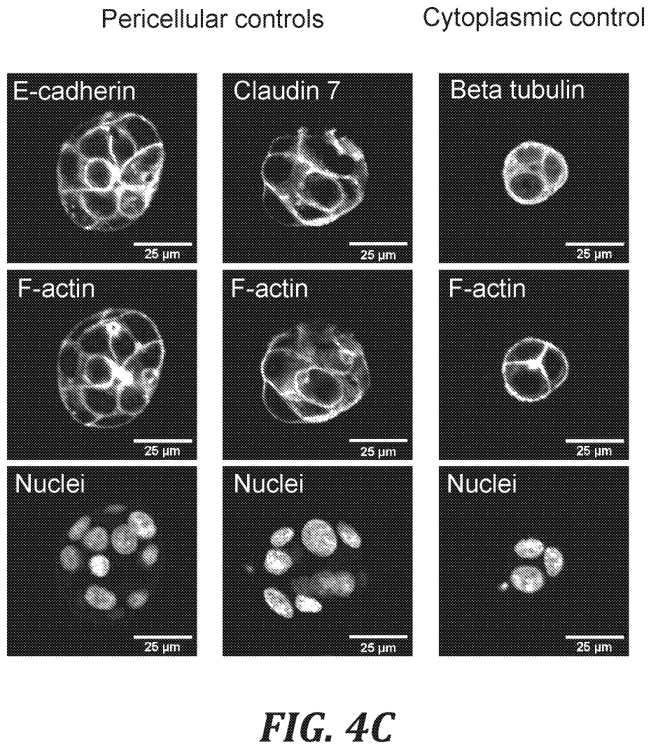

The recovery of Claudin 7 was used to evaluate the efficiency with which full-length intercellular proteins were released from the magnetic bead. Claudin 7 is a well-known tight-junction protein associated with intercellular adhesions (FIG. 4A), and it is localized to the pericellular zones in HCC70 spheroids (FIG. 4D). Next, western blots against Claudin 7 were performed in the eluted fraction from the streptavidin pull-down, and the signal intensity of Claudin 7 from the 15 μg of HCC70 spheroid starting lysate was evaluated. The DBCO-PEG4-desthiobiotin-labeled samples exhibited, on average, a 10-fold higher recovery of Claudin 7 compared to the DBCO-sulfo-biotin samples (FIGS. 4A and 4B). Moreover, this superior recovery was achieved in milder conditions that lead to greater compatibility with downstream detection strategies.

The next aim was to evaluate the extent to which intercellular proteins were enriched, and cytoplasmic proteins were depleted, in the eluted fraction produced using the DBCO-PEG4-Desthiobiotin labeling scheme. GAPDH and Beta-tubulin, two cytoplasmic proteins, were chosen as controls, as both are known to be intercellularly localized. control E-cadherin and Claudin 7, two well-known transmembrane proteins, were also chosen as control proteins. Additionally, immunofluorescence confocal microscopy using antibodies against E-cadherin Claudin 7, and Beta-tubulin confirmed each protein's expected spatial localization (FIG. 4D). Western blot analysis of the eluted fraction of each of these proteins were performed directly alongside a 15 pg sample of the crude lysate, to quantify the ratio of pericellular proteins to cytosolic proteins in each sample (Claudin7:GAPDH and E-cadherin:Beta-tubulin). The eluted fraction obtained from the enrichment procedure in the samples treated with DBCO-PEG4-Desthiobiotin exhibited significantly higher ratios of the pericellular control proteins compared to the cytosolic control proteins in the crude lysate (FIG. 4C). These results demonstrate that this embodiment of the invention enriches pericellular proteins and depletes intracellular proteins.

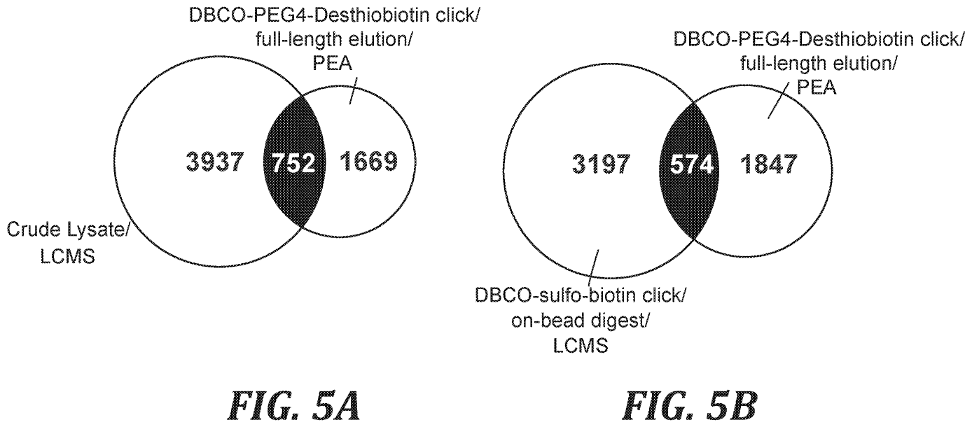

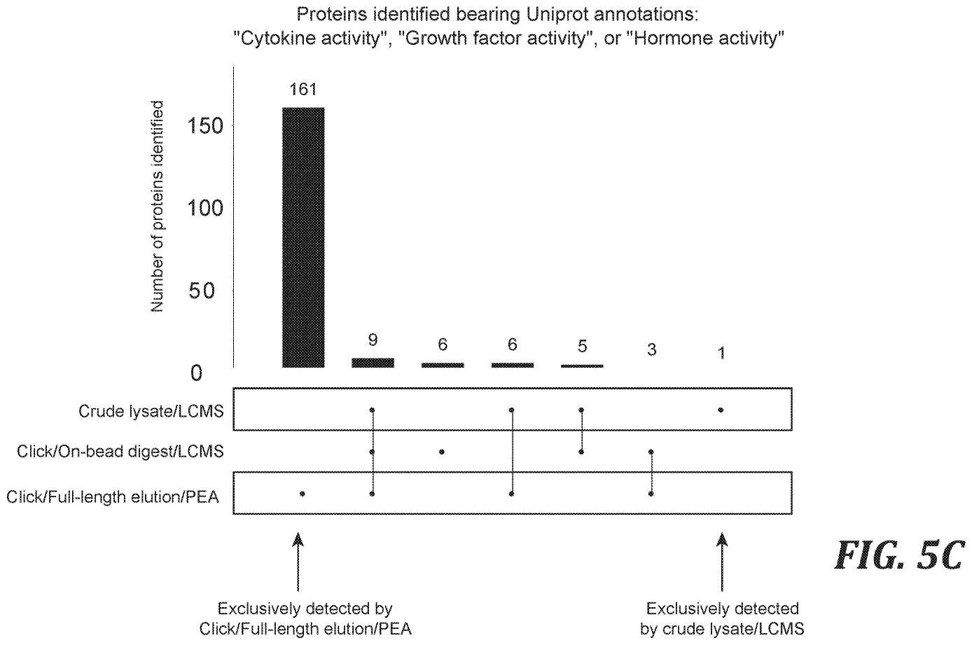

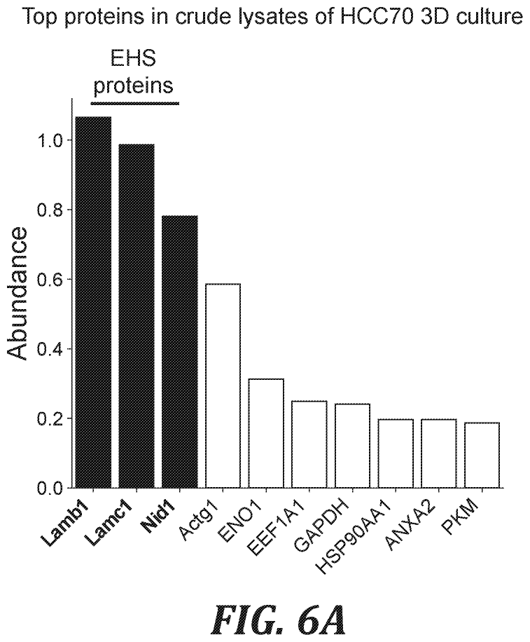

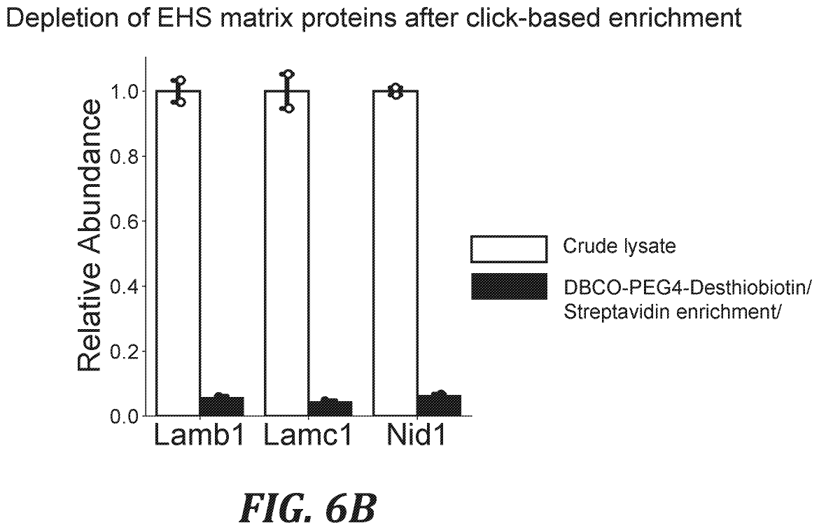

Additionally, the next aim was to confirm that the eluted fraction obtained from streptavidin bead enrichment of the DBCO-PEG4-Desthiobiotin labeled samples was significantly depleted in EHS matrix proteins. Compositions of the EHS matrix vary from lot-to-lot, but three of the most abundant proteins EHS matrix preparations are known to be Laminin B1, Laminin C1, and Nidogen. LC/MS proteomics were performed on crude lysates prepared from organotypic cultures of HCC70 spheroids in 2% EHS matrix. As expected, the most abundant proteins in these crude lysates were the EHS matrix proteins, Laminin B1, Laminin Cl, and Nidogen (FIG. 5A). This was true regardless of whether the peptide search was performed against the human or mouse proteome, due to high sequence homology of these proteins between both species.

The expectation was that the eluted fraction from the proteins enriched using the DBCO-PEG4-Desthiobiotin would be significantly depleted in these EHS matrix proteins. Thus, the pericellular proteome of HCC70 spheroids was enriched in organotypic cultures containing 2% EHS matrix using the azidohomoalanine/DBCO-PEG4-Desthiobiotin labeling procedure, followed by streptavidin magnetic bead enrichment and competitive elution with free biotin. LC/MS proteomics were performed on the eluted fraction and directly compared to the lysates that were used as input for the streptavidin bead enrichment. After performing label-free-quantification and normalization, the samples that had undergone the click labeling procedure and streptavidin enrichment exhibited over 10-fold lower normalized abundance of Laminin B1, Laminin Cl, and Nidogen in comparison to the starting lysates (FIG. 5B). Therefore, this embodiment of the disclosure provides substantial depletion of exogeneous proteins in comparison to crude lysate preparations.

Pericellular click labelingwithDBCO-PEG4-Desthiobiotin andfull-length protein elution enables discovery of pericellular cytokines, growth factors, and other proteins that are undetectable by LCMS. Bottom-up, shotgun proteomics using liquid-chromatography mass-spectrometry (LC/MS) is a widely used approach for discovery proteomics in biological samples. Because LC/MS protocols involve a tryptic digestion step to produce peptide fragments, and conventional biotin-streptavidin based enrichment approaches require an on-bead tryptic digestion step for efficient recovery of immobilized proteins, LC/MS protocols are a common method for proteomic analysis following biotin-streptavidin enrichment. However, LC/MS is limited in its ability to detect proteins that are low in relative abundance in complex mixtures. Proteins containing transmembrane domains (as are common for growth factors and cell surface proteins) can also present a challenge, since LC/MS relies on generation of tryptic peptides using proteases that recognize hydrophilic residues. This limitation renders the detection of growth factors, cytokines, and hormones particularly challenging, because they typically are significantly less abundant than major structure proteins (such as cytoskeletal and extracellular matrix proteins) and generate fewer tryptic peptides that are suitable for downstream LC/MS. LC/MS proteomics of crude lysates prepared from HCC70 3D cultures identified only 15 proteins bearing Uniprot annotations of “Growth factor activity,” “Cytokine activity,” or “Hormone activity.” This presents a challenge, because proteins such as growth factors, cytokines, and hormones are highly relevant to intercellular communication.

In comparison, full-length proteins are necessary for reliable identification and quantification in immunoaffinity-based detection methods (such as ELISAs). Such methods are more readily amenable to quantification of growth factors, cytokines, and hormones, due to their high sensitivity and ability to target pre-selected candidate proteins within a complex mixture. Thus, the ability to enrich full-length proteins expands the scope of available downstream analytical approaches that can be used in tandem with the enrichment procedure. The expectation was that combining the bioorthogonal chemistry-based enrichment procedure with a high-throughput immunoaffinity-based multiplex panel would enable the identification and quantification of growth factors, cytokines, hormones, and other proteins in pericellular spaces that would not be detected by LC/MS, whether performed on crude lysates or in tandem with on-bead digestion after immobilization on a biotin-binding solid support.

Thus, the aim was to evaluate whether DBCO-PEG4-Desthiobiotin labeling and full-length protein elution could be used in combination with the proximity extension assay (PEA), specifically the Olink® Explore 3072 protocol. This PEA quantification method uses an antibody panel targeting up to 3072 unique protein targets, wherein two different antibodies are used for each target. Each antibody is covalent attached to an oligonucleotide, which has been engineered to hybridize to the cognate oligonucleotide that is attached to the antibody partner that binds to the same protein target. Antibody pairs that bind to the same target will result in hybridization with their complementary oligonucleotides, which enables a polymerase chain reaction (PCR) amplification. The amplified products are then quantified by next generation sequencing to quantify on-target events in which two members of an antibody pair bind to the same target. In this way, false positives are reduced in comparison to simple multiplex antibody arrays, because off-target proteins are less likely to bind to two separate antibodies raised against separate antigens on a given target.

Enriched pericellular protein fractions were generated from HCC70 organotypic cultures in 2% EHS matrix by sequential labeling with azidohomolanine and DBCO-PEG4-Desthiobiotin, followed by enrichment with streptavidin magnetic beads and competitive elution with free biotin. The enriched fraction and the unbound fraction were used directly as input into the Olink Explore protocol. After normalization, proteins below the lower detection limit were discarded, and a list of proteins, together with their relative abundance, in the enriched fraction was obtained. For comparison to LC/MS without pericellular protein enrichment, LC/MS proteomics was also performed on crude lysates of HC770 3D cultures. Additionally, to evaluate the benefit of full-length protein elution rather than on-bead digestion, pericellular proteins from HCC70 cells were labeled with DBCO-sulfo-Biotin, immobilized with streptavidin magnetic beads, and then an on-bead digestion with proteases was performed to release peptides for downstream LC/MS analysis. Using the click enrichment procedure in tandem with the Olink® Explore protocol, 1,669 proteins were identified that were not detected using LC/MS of crude lysates. Also, 1,847 proteins were identified using the click enrichment procedure and the Olink® Explore protocol that were not identified using the on-bead digest approach.

The next aim was to evaluate the extent to which the 1,669 proteins that were uniquely identified using the click enrichment approach included growth factors, cytokines, and hormones. The list of proteins identified by the click enrichment/Olink Explore method and the LC/MS proteomics of crude lysates were cross-referenced against the Uniprot KB database for proteins bearing one of the following annotations: “Growth factor activity,” “Cytokine activity,” or “Hormone activity.” Among 191 proteins in the Uniprot KB database containing at least one of these annotations that were detected by at least one of the three techniques, 161 were detected exclusively with click/Olink method. In comparison only one protein was detected exclusively by LC/MS of crude lysates, and six proteins were detected exclusively using the on-bead digestion approach. Thus, of all growth factors, cytokines, and hormones detected in at least one of the three approaches, 84% were identified exclusively using the specific embodiment of the invention utilizing azidohomoalanine, DBCO-PEG4-desthiobiotin labeling, full length protein elution, and PEA quantification (by Olink), and not detected using LC/MS of crude lysates, or conventional on-bead digestion.

### Example 1 Materials and Methods

Click chemistry and biotinylation reagents. L-Azidohomoalanine, DBCO-sulfo-Biotin, and DBCO-PEG4-Desthiobiotin were purchased from Click Chemistry Tools (cat. #1066-100, #A116-25, #1108-25, respectively). Sulfo-NHS-Biotin was purchased from Thermo Fisher Scientific (cat. #A39256) and was not reconstituted until immediately before use. L-Azidohomoalanine was reconstituted in anhydrous DMSO at 500 mM. DBCO-sulfo-biotin and DBCO-PEG4-Desthiobiotin were reconstituted in anhydrous DMSO at 300 mM. All labeling reagents were stored at −20° C. under desiccated conditions.

Antibodies andfluorescent labels. Streptavidin-alexa-fluor 488 conjugate was purchased from Thermo Fisher Scientific (cat. #S11223). The lyophilized powder was reconstituted at 1 mg/mL in PBS, and the reconstituted solution was used at a 1:1,000 dilution. Claudin 7 was detected by IF (1:100 dilution) and WB (1:1,000 dilution) using a rabbit polyclonal antibody against human Claudin 7 (Thermo Fisher Scientific #34-9100). E-cadherin was detected IF (1:100 dilution) and WB (1:1,000 dilution) using a rabbit monoclonal antibody against human E-cadherin (Cell Signaling Technology #3195S). GAPDH was detected by WB (1:1,000 dilution) using a mouse monoclonal antibody against human GAPDH (ProteinTech #60004). Beta-tubulin was detected by IF (1:100 dilution) and WB (1:1,000 dilution) using a rabbit polyclonal antibody against human Beta-tubulin (Abcam #ab6046). Secondary antibody fluorescent labeling for indirect immunofluorescence microscopy was performed using one of the following fluorescently conjugated secondary antibodies (1:500 dilution), raised against the corresponding host species of the primary antibody: Goat anti-rabbit 488 polyclonal antibody (Thermo Fisher Scientific #A-11034), Goat anti-mouse 488 polyclonal antibody (Thermo Fisher Scientific #A1 1029). Secondary antibody fluorescent detection for western blots and dot blots was detected using one of the following secondary antibodies (1:10,000 dilution): Goat anti-rabbit IRDye 800CW (LiCor #925-32211), Goat anti-mouse IRDye 800CW (LiCor #925-32210), Donkey anti-mouse IRDye 680RD (LiCor #925-68072), Goat anti-rabbit IRDye 680RD (LiCor #926-68071). Streptavidin IRDye 800CW (LiCor #92632230).

HCC70 cells. HCC70 cells were purchased from the American Type Culture Collection (ATCC).

HCC70 2D monolayer culture. Prior to generation of 3D spheroid cultures, HCC70 cells (purchased from ATCC) were maintained and propagated in conventional 2D monolayer culture. Cells were cultured on TC-treated polystyrene at 37° C. in sterile tissue-culture incubators at 5% CO₂. HCC70 cells were cultured in RPMI media containing 10% fetal bovine serum, 1% penicillin-streptomycin, 2 mM GlutaMax, and 1 mM sodium pyruvate. Media was replaced with fresh cell culture media every 3 days, and cells were passaged once per week by dissociation with TrypLE express, followed by 1:4 dilution into fresh TC-treated culture flasks. Cells were routinely evaluated for mycoplasma and verified to be negative.

Preparation of 2% EHS matrix suspension media. 2% EHS matrix suspension media was obtained by combining ice-cold EHS matrix (Cultrex reduced growth factor basement membrane extract, R&D systems #3433-005-01) with ice-cold complete cell culture media (RPMI media containing 10% fetal bovine serum, 1% penicillin-streptomycin, 2 mM GlutaMax, and 1 mM sodium pyruvate) at a ratio of 1:49 (for example, 1 mL of EHS matrix at the manufacturer's delivered concentration into 49 mL complete cell culture media). The mixture was immediately mixed well by inverting, and the EHS-supplemented media was then pre-warmed at 37° C. in a water bath to allow the EHS matrix to form crosslinks. A methionine-reduced variation of 2% EHS matrix was prepared as above, except that RPMI media was replaced with methionine-free RPMI (Gibco #A1451701).

Generation of HCC70 3D spheroid cultures in 2% EHS matrix. Prior to generation of 3D spheroid cultures, HCC70 cells were passaged twice after revival from liquid nitrogen cryostorage to minimize effects associated with thawing. HCC70 cells were dissociated from 2D TC-treated culture flasks using TrypLE express by incubation for about 5 minutes at 37 degrees Celsius, or until cells fully detached from the surface. The cells were recovered from the flask and diluted 5:1 with complete cell culture media (RPMI, 10% FBS, 1% penicillin-streptomycin, 2 mM GlutaMax, 1 mM sodium pyruvate). Cells were pelleted by centrifugation at 200×g for 3 minutes and gently resuspended in phosphate buffered saline (PBS) without calcium or magnesium. Cells were counted with an automated cell counter hemocytometer (Countess). Next, the desired number of cells was pelleted by centrifugation at 200×g for 3 minutes and cells were resuspended in 2% EHS suspension media (prepared according to the recipe above) at a cell density of 150 thousand cells per mL of suspension media. The cell suspension was plated in 6-well ultra-low attachment plates (Corning #3471), and multicellular clusters were allowed to form. Spheroids were dissociated once after 4 days of initial establishment before being plated for experiments.

To dissociate spheroids, spheroids were pelleted by centrifugation at 200×g for 3 minutes and washed twice with PBS (without calcium, magnesium). Cells were then resuspended in Accumax cell dissociation solution (Innovative Cell Technologies #AM105) and incubated in a 37° C. water bath for 5 minutes. The spheroids were triturated with a 5 mL serological pipette ten times. The spheroids were then incubated at room temperature in the dissociation solution, and every 5 minutes the suspension was triturated with a 1 mL micropipette ten times. This process was repeated for up to 30 minutes until at least 80% of the cells were observed to be a single cell suspension when visualized under a phase contrast microscope.

Following dissociation, cells were pelleted at 200×g for 3 minutes and resuspended in PBS (without calcium, magnesium). Cells were counted on a hemocytometer using an automated cell counting system (Countess) and resuspended in 2% EHS matrix suspension media (recipe described above) at a cell density of 150 thousand cells per mL. The cell suspension was plated in 6-well ultra-low attachment plates (Corning #3471), and multicellular clusters were allowed to form.

Generation of fully embedded 3D HCC70 cultures. HCC70 spheroids were first seeded in suspension in 2% EHS matrix (following one passage in 3D suspension culture). After allowing spheroids to form for 24 hours, the spheroids were pelleted, the supernatant was aspirated, and the spheroids were directly resuspended in ice-cold EHS matrix (undiluted from the manufacturer's preparation at a typical concentration of 7-10 mg/mL). Resuspension was achieved by slow pipetting up and down forty times on ice with pipette tips that were pre-chilled at −20° C. Next, EHS matrix “domes” were created by pipetting thirty microliters of the EHS matrix/spheroid suspension directly into the center of the well of a 24-well plate with a No. 1.5 polymer coverglass bottom. The plate was flipped upside down to prevent spheroids from settling onto the coverglass, and the plate was incubated at 37° C., 5% CO₂, for 1 hour to allow the EHS matrix to form an elastic hydrogel. The domes were then covered with complete cell culture media (1 mL per well) and cultured at 37° C., 5% CO₂.

Azidohomoalanine labeling of 3D HCC70 cultures in 2% EHS matrix. 3D suspension cultures of HCC70 cells in 2% EHS matrix were generated from 2D monolayer culture (as described above), dissociated once, and then plated in fresh 2% EHS matrix. After 48 hours of culture, spheroids were washed three times to remove excess methionine by centrifuging at 200×g for 3 minutes, aspirating the supernatant and resuspending in PBS (with calcium and magnesium). Azidohomoalanine containing media was freshly prepared by diluting a 500 mM azidohomoalanine DMSO stock solution 1,000-fold in methionine reduced 2% EHS matrix suspension media. The washed spheroids were resuspended in azidohomoalanine-containing 2% EHS matrix suspension media (in the same volume as the original suspension), plated in 6-well ultra-low attachment plates, and incubated in TC incubator at 37° C., 5% CO₂ for 24 hours.

DBCO-PEG4-Desthiobiotin and DBCO-sulfo-Biotin labeling in 2% EHS matrix suspension culture. Spheroids were cultured for 24 hours in the presence of azidohomoalanine to allow the azidohomoalanine to be incorporated into newly synthesized proteins at methionine residues. After 24 hours, spheroids were washed three times with PBS (containing calcium and magnesium) to remove unincorporated azidohomoalanine by centrifugation at 200×g for 1 minute, aspiration of the supernatant, and resuspension in the PBS wash buffer. The spheroids were then resuspended in a PBS solution containing 1% bovine serum albumin and the heterobifunctional click reagent at a concentration of three hundred micromolar. The cells were incubated at 37° C. in 6-well ultra-low-adhesion plates for 30 minutes in a tissue culture incubator maintained at 5% CO₂ to allow the SPAAC reaction to proceed. Excess unreacted reagent was then removed by washing the cells four times with PBS (containing calcium and magnesium) by repeated centrifugation at 4° C. (200×g, 1 minute) and resuspension.

Sulfo-NHS-Biotin labeling in 2% EHS matrix suspension culture. HCC70 spheroids were cultured for 48 hours in methionine-containing 2% EHS matrix suspension media. Spheroids were then washed three times with PBS (containing calcium and magnesium) by centrifugation at 200×g for 3 minutes, followed by resuspension in the PBS wash buffer. Sulfo-NHS-Biotin concentrated stock was freshly prepared at 10 mM by dissolving in anhydrous DMSO. Sulfo-NHS-biotin labeling buffer was freshly prepared by diluting the 10 mM DMSO stock solution in PBS (containing calcium and magnesium, pre-warmed to 37° C.) to a final concentration of five hundred micromolar. The sulfo-NHS-biotin labeling buffer was used immediately to prevent premature hydrolysis of the probe by resuspending the pelleted spheroids directly in the labeling buffer. The spheroids were then plated into 6-well ultra-low attachment plates and incubated at 37° C., 5% CO₂ in a tissue-culture incubator for 30 minutes. The spheroids were then washed two times with ice-cold PBS containing calcium, magnesium, and 200 mM glycine to remove unreacted probe and quench unreacted NHS reagent by centrifuging at 4° C., 200×g for 1 minute each, followed by resuspension in the wash buffer. Spheroids were then washed once with ice-cold PBS containing calcium, magnesium (no glycine) by centrifuging at 4° C., 200×g for 1 minute and resuspending in ice-cold PBS.

Spheroid fixation and streptavidin fluorescence detection of biotin or desthiobiotin labeledsamples. Immediately following labeling with biotin or desthiobiotin containing probes, the spheroids were pelleted by centrifugation (200×g, 1 minute) and resuspended the spheroids in a fixation solution consisting of PBS, 4% paraformaldehyde, and 0.1% glutaraldehyde. The spheroids were incubated in fixation solution for 10 minutes at room temperature with gentle agitation on an orbital shaker. After 10 minutes of incubation, the spheroids were allowed to settle by gravity, removed excess fixation solution, and washed the spheroids once with a quenching buffer consisting of PBS and 200 mM glycine. After removing excess quenching solution, the spheroids were then resuspended a second time in the quenching buffer and incubated the spheroids for 20 minutes at room temperature to quench unreacted aldehydes.

To prepare the spheroids for secondary detection of biotin or desthiobiotin, the cell membrane was permeabilized for 1 hour at room temperature with a solution of PBS containing 0.1% Tween-20. Next, non-specific binding was blocked using a solution of PBS containing 0.1% Tween-20 and 10% FBS for 1 hour at room temperature.

Proteins that had undergone a labeling reaction were detected using Alexafluor 488-conjugated streptavidin (streptaviding-488). Samples were incubated with a solution of PBS containing streptavidin-488 at a concentration of 0.2 mg/mL (1,000× dilution of a 2 mg/mL stock) and 0.1% (v/v) Tween-20 for 16-24 hours at 4° C. Samples were then washed for 10 minutes with PBS containing 0.1% tween-20, 4 times. Spheroids were counterstained to label nuclei and F-actin with DAPI and phalloidin-568 in PBS with 0.1% Tween-20 for 1 hour at room temperature. Samples were then washed for 10 minutes with PBS containing 0.1% Tween-20, 4 times. Finally, samples were allowed to settle by gravity, the wash buffer was aspirated, and spheroids were suspended in PBS (containing calcium, magnesium) and stored at 4° C. for up to 4 days prior to imaging.

Indirect immunofluorescence staining of pericellular or cytosolic control proteins. HCC70 spheroids were prepared in 2% EHS suspension culture (following one passage in 3D culture). 72 hours after plating, spheroids were pelleted by centrifugation at 200×g for 3 minutes and resuspended in a fixation buffer of 4% paraformaldehyde (PFA) in PBS (with calcium and magnesium). Spheroids were incubated in fixation solution for 10 minutes at room temperature in 24-well plates with No. 1.5 coverglass bottoms. After fixation, spheroids were allowed to settle by gravity, the fixative was aspirated, and spheroids were washed once with PBS containing 200 mM glycine. After settling by gravity and resuspension, spheroids were resuspended in the PBS/glycine quenching buffer and incubated for 20 minutes at room temperature with gentle agitation. The spheroids were then washed four times with PBS (with calcium, magnesium) by resuspension in the wash buffer, incubation for 10 minutes, gravity settling, and aspiration of the wash buffer.

Spheroids were permeabilized for 1 hour with PBST (PBS, 0.1% Tween-20, with calcium, magnesium), then blocked for 1 hour with PBST containing 10% normal goat serum. Primary antibody labeling solution was prepared by diluting primary antibodies 1:100 in a buffer of PBS, 0.1% (v/v) Tween-20, 0.1% (w/v) BSA. Spheroids were allowed to settle by gravity and resuspended in antibody labeling buffer, then incubated for 16-20 hours at 4° C. with gentle orbital agitation. Samples were washed four times, 10 minutes each, with PBST by gravity settling and resuspension. Secondary antibody labeling solution was prepared by diluting secondary antibodies 1:500 in a buffer of PBS, 0.1% (v/v) Tween-20, 0.1% (w/v) BSA. Samples were incubated in secondary antibody labeling buffer for 16-20 hours at 4° C. with gentle orbital agitation. Spheroids were washed four times with PBST, 10 minutes each, then counterstained with DAPI and phalloidin-alexafluor568 in PBST for 1 hour. Samples were washed twice with PBST and twice with PBS, 10 minutes each. Spheroids were maintained in PBS at 4° C. and were used within 4 days for imaging by confocal fluorescence microscopy.

Confocal microscopy. Samples were plated into 24-well plates containing a No. 1.5 polymer coverglass. Confocal mages were acquired using an Andor CSU-W confocal spinning disk on a Leica DMi8 inverted microscope using a 40X water immersion objective.

Preparation of spheroid lysates following labeling in 2% EHS matrix. Immediately following the above labeling with either sulfo-NHS-biotin, DBCO-sulfo-Biotin, or DBCO-PEG4-Desthiobiotin, spheroids were pelleted at 200×g for 3 minutes at 4° C. Excess supernatant was aspirated, and the spheroid pellets were flash frozen by submerging in liquid nitrogen for 5 minutes. The spheroid pellets were then stored at −80° C. until ready for use.

Prior to thawing spheroid samples, a lysis buffer consisting of 0.1% SDS, 1% Triton X-100, 150 mM NaCl, 20 mM Tris, 1 mM EDTA, pH 7.4 was freshly prepared and supplemented with protease inhibitors (Halt Protease inhibitor cocktail, 100× dilution, Thermo Fisher Scientific #87785) and phosphatase inhibitors (Halt Phosphatase inhibitor cocktail, 100× dilution, Thermo Scientific #78420). Spheroid pellets were thawed on ice in direct contact with ice-cold lysis buffer (one hundred microliters of lysis buffer per one million cells seeded for the spheroids). The mixture was periodically vortexed to facilitate thawing until the pellet was fully thawed. Once the pellets were fully thawed, samples were briefly vortexed to fully resuspend the cells, and samples were sonicated using a probe sonicator with six pulses, 0.5 seconds each. Samples were then incubated with gentle orbital agitation at 4° C. for 1 hour. After incubation, samples were again sonicated using a probe sonicator with six pulses, 0.5 seconds each. Next, samples were centrifuged in a pre-chilled microcentrifuge at 4° C. at 12,000×g for 10 minutes to remove insoluble material. The supernatant (representing the cleared spheroid lysate) was collected and used for the next step of the enrichment.

Streptavidin magnetic bead enrichment. One micron-sized magnetic polystyrene beads covalently functionalized with streptavidin (binding potential: 2,500 pmoles free biotin/mg beads), bearing a hydrophilic, carboxylated surface (Streptavidin MyOne Cl, Thermo Fisher cat #65001), and a magnetic separation rack (DynaMag 2, Thermo Fisher Scientific cat #12321D) were used to enrich proteins that had been covalently labeled with either a biotin or desthiobiotin enrichment handle. For every 100 μL of sample, 200 μL of the 10 mg/mL magnetic bead suspension were used. Prior to use, the beads were washed 3 times with a tris-buffered saline wash buffer consisting of 150 mM NaCl, 20 mM Tris, 0.2% Tween-20, pH 7.6 (from here, referred to as “TBST”) by separating the beads on the magnetic stand, removing the supernatant, and resuspending the beads in 500 μL of TBST. Next a magnetic bead pellet was prepared by separating the beads on a magnetic stand and removing the supernatant. The cleared spheroid lysate was added directly to the magnetic bead pellet, and the beads were resuspended in the lysate by dragging the tube along a microtube rack repeatedly and relying on the vibrations generated by the uneven microtube rack surface (This method of dragging the tube along the microtube rack was found to be superior to vortexing). The bead/lysate mixture was then incubated for 3 hours at room temperature with orbital agitation to prevent the beads from settling. Next, the magnetic beads were separated on the magnetic stand, and the supernatant was collected and kept as the “unbound fraction.” The beads were then washed three times with TBST by repeatedly resuspending the pellet (by running the tube along a microtube rack) and then separating the magnetic beads. No incubation time was applied during the washes.

To elute bound proteins from the beads, separate approaches were used for the DBCO-sulfo-biotin labeled samples and the DBCO-PEG4-desthiobiotin labeled samples. To elute bound proteins from the beads in the DBCO-sulfo-biotin labeled samples, the beads were resuspended in a tris-buffered-saline solution containing 8 M urea, 2% sodium dodecyl sulfate, and 3 mg/mL biotin; and incubated the samples at 70° C. for 1 hour. 50 μL of elution buffer were used per 100 μL of starting lysate material. The beads were then separated on a magnetic stand, and the supernatant was collected as the final elution fraction.

To elute bound proteins from the beads in the DBCO-PEG-Desthiobiotin labeled samples, an elution buffer containing 0.1% SDS, 1% Triton X-100, 150 mM NaCl, 20 mM Tris, 1 mM EDTA, 3 mg/mL biotin was prepared. Biotin free acid is only soluble in water in its deprotonated form, so a 2× concentrated form of the buffer was initially prepared and titrated with sodium hydroxide until all biotin fully dissolved and the pH reached 7.4-7.6. The buffer was then diluted with MilliQ water to reach the composition.

100 μL of elution buffer were used per 100 μL of starting lysate material. The pelleted beads, containing the immobilized proteins, were resuspended in the elution buffer by dragging the tube along a microtube holder. The samples were first incubated for 2 hours at 37° C. while shaking at 200 RPM, followed by a 16-hour incubation at 4° C., with gentle orbital agitation. Next, the magnetic beads were separated on a magnetic stand, and the supernatant was collected as the final elution fraction.

Streptavidin dot blot analysis. Spheroid lysates were prepared as described above. 5 μL (containing approximately 20 μg of total protein lysate) of lysate solutions was slowly pipetted into a single spot onto a 0.45-micron nitrocellulose membrane (Thermo Scientific #88014), allowing the sample to wick into the membrane. The membrane was blocked with a solution of 3% BSA in TBS (20 mM Tris, 150 mM NaCl, pH 7.6) for 1 hour at room temperature. A streptavidin labeling solution was freshly prepared by diluting IRDye 800CW Streptavidin to a final concentration of 0.1 pg/mL into a buffer of 20 mM Tris, 150 mM NaCl, 0.2% Tween-20, 0.01% sodium dodecyl sulfate, pH 7.6. The membrane was incubated in streptavidin labeling buffer for 1 hour at room temperature. The membrane was washed four times with TBST (20 mM Tris, 150 mM NaCl, 0.2% Tween-20, pH 7.6) for 5 minutes each, followed by three rinses with TBS (20 mM Tris, 150 mM NaCl, pH 7.6). The fluorescence intensity was then assessed by fluorescence IR imaging with a LiCor Odyssey CLx.

SDSPAGE Western Blotting. Samples were prepared for western blotting by combining the desired volume of elution or lysate with Bolt reducing agent (10X dilution, Thermo Fisher #B0007), Bolt LDS sample buffer (4X dilution, Thermo Fisher #B0007), and MilliQ water to bring the final volume to between 30-35 microliters per lane to be loaded. The samples were denatured by incubating at 70° C. for 10 minutes.

Polyacrylamide gel electrophoresis on the samples was performed using the Bolt system (Invitrogen). The samples were loaded into wells of a Bolt 4 to 12% gradient Bis-Tris polyacrylamide, 1 mm thick hydrogels (Thermo Fisher #NWO4122BOX), and electrophoresis was performed in a Bolt MES SDS running buffer (Thermo Fisher #B0002) that was supplemented with Bolt antioxidant (500× dilution, Thermo Fisher #BT0005). Electrophoresis was performed in two steps: one step at 100 V for 30 minutes, followed by a step at 170V for 20 minutes, or until the loading dye was observed to reach the bottom of the gel.

Proteins were then transferred from the polyacrylamide gel to 0.2-micron PVDF membranes (Millipore #SEQ08130). Specifically, the PVDF membrane was pre-wetted with methanol for 1 minute, then washed three times with MilliQ water. The membrane was then soaked for 5 minutes in Bolt western blot transfer buffer, which was prepared by diluting 20× concentrated Bolt transfer buffer (Thermo Fisher #BT0006). A western blot sandwich was prepared consisting of blot filter paper-Blotting sponge pads-Polyacrylamide gel-PVDF membrane-blot sponge-blot filter paper, and transfer to the PVDF membrane was performed in the Bolt transfer buffer at 15 V for 1 hour.

After transfer to the PVDF membrane, the membrane was washed three times with MilliQ water, 5 minutes each. The membrane was blocked with a solution of 3% (w/v) bovine serum albumin (BSA) in tris-buffered saline (20 mM Tris, 150 mM NaCl, pH 7.6) for 1 hour with gentle orbital agitation. Primary antibodies where then diluted 1,000 times in a primary antibody staining buffer (20 mM Tris, 150 mM NaCl, 0.2% Tween-20, pH 7.6), and the PVDF membrane was incubated in antibody staining buffer for 16-20 hours at 4° C. with gentle orbital agitation.

The PVDF membrane was washed three times in TBST wash buffer (20 mM Tris, 150 mM NaCl, 0.2% Tween-20, pH 7.6), 5 minutes each with gentle orbital agitation. For simultaneous detection of two targets labeled with primary antibodies from different host species, one target was detected with a secondary antibody conjugated to a IRDye 680RD fluorophore, and the other target was detected using a secondary antibody conjugated to an IRDye 800CW fluorophore. Alternatively, when more than 2 targets were detected on the same blot, and at least 2 targets were detected using primary antibodies from the same host species, the PVDF membrane was cut prior to staining with the secondary antibody to segregate the membrane into distinct regions for staining targets that were well-separated by apparent molecular weight. To determine the appropriate position of the cuts, a western blot was first performed with each target of interest on a separate blot, and the appropriate molecular weight reference positions were determined that would fully encompass all bands. The cuts on the blot were then made based using the protein MW ladder as a reference, and each portion of the membrane containing the desired targets was stained separately.

Secondary antibodies were diluted 10,000 times in secondary antibody incubation buffer (20 mM Tris, 150 mM NaCl, 0.2% Tween-20, 0.01% SDS, pH 7.6). The PVDF membrane was incubated with secondary antibodies for 1 hour at room temperature with gentle orbital agitation. The membrane was then washed 3 times with TBST (20 mM Tris, 150 mM NaCl, 0.2% Tween-20, pH 7.6) for 5 minute each, followed by 3 washes with TBS (20 mM Tris, 150 mM NaCl, pH 7.6) in which the TBS was added and immediately removed to wash off excess Tween-20.

PVDF membranes were then imaged using IR fluorescence imaging with a LiCor Odyssey CLx. The IRDye 680RD fluorophore was detected using the 700 nm channel and the IRDye 800CW fluorophore was detected using the 800CW channel. Membranes were imaged at 169-micron resolution.

Western blot and dot blot quantification. Western blot quantification was performed using ImageJ. A rectangular region of interest (ROI) was created around each band, cropping it as close as possible to the portion of the band that exhibited measurable signal above the surrounding background. For each band, a corresponding background ROI was created to measure the background fluorescence intensity of a band of equal size in a region of the gel that was observed to exhibit only non-specific background staining. Integrated fluorescence intensity was calculated using the “Measure” tool, and the background-corrected integrated fluorescence intensity was computed for each band.

Discovery and quantification of protein abundance using the proximity extension assay. Proteins were measured using the Olink® Explore 3072 panel (Olink Proteomics AB, Uppsala, Sweden) according to the manufacturer's instructions. For each experimental replicate, eighty microliters of elution solution (representing labeled proteins from eight hundred thousand cells), was used directly for the Olink Explore protocol. The Olink Explore 3072 panel uses a 3072-plex panel of oligonucleotide-conjugated antibody probe pairs. Each antibody probe pair is designed to bind to the target, resulting in hybridization of the complementary oligonucleotides provided by each member of the paper. DNA polymerase is then used to extend only oligonucleotides that have hybridized, producing a unique DNA barcode each time a protein is bound to both members of an antibody pair whose conjugated oligonucleotides were designed to hybridize. Library preparation was then performed to add sample identification indices and the necessary nucleotides for illumine sequencing. Libraries were subjected to a bead-based purification and quality was assessed using an Agilent 2100 bioanalyzer (Agilent Technologies, Palo Alto, CA). Samples were then subjected to next-generation sequencing using the Illumina NovaSeq 6000. The data was then quality controlled and converted into Normalized Protein eXpression (NPX) values, which represent Olink's unit of relative abundance. For quality control, three internal controls were spiked into each sample and used to evaluate the performance of 3 steps in the protocol: an incubation control with a non-human antigen and its matching antibody probes, an extension control consisting of an IgG antibody conjugated to a pair of hybridizing oligonucleotides, and an amplification control consisting of a complete double-stranded DNA amplicon. The Olink Explore protocol was also performed on a set of external controls: two different pooled human plasma samples, three negative controls, and three plate controls. Each plate control is a plasma sample that is used to normalize across plates.

The NPX value was calculated by first computing ExtNPX(assay I, sample J), according to ExtNPX(assay i, sample j)=log 2(counts(assay i, sample j)/(counts(Extension control, sample j)); which normalized the counts of each sample to the counts of a known standard. Next the NPX value was computed as NPX(assay i, sample j)=ExtNPX(assay i, sample j)−median (ExtNPX(PC, assay i)); which normalized across plates. The limit of detection for each sample was defined to be equal to an NPX value that is three standard deviations above the median of the negative control samples. All proteins below the LOD were excluded from the list of discovered proteins. All assay validation data (detection limits, intra- and inter-assay precision data, predefined values, etc.) are available on manufacturer's website.

Preparation of crude lysate gel slices for LC/MS. Protein concentration of crude lysates was determined using a Pierce BCA assay kit (Thermo Fisher Scientific #23225) according to the manufacturer's protocol. 20 μg of crude lysate was then denatured (as described in the methods for western blotting) and samples were loaded into wells of a Bolt 4 to 12% gradient Bis-Tris polyacrylamide, 1 mm thick hydrogels (Thermo Fisher #NWO4122BOX), and electrophoresis was performed in a Bolt MES SDS running buffer (Thermo Fisher #B0002) that was supplemented with Bolt antioxidant (500× dilution, Thermo Fisher #BT0005). Electrophoresis was performed at 100 V for 5-10 minutes, or until the loading dye had traversed about 2 cm from the foot of the well.

The polyacrylamide gel was washed three times with MilliQ water and then stained for 1 hour with Coomassie G-250 using the SimplyBlue™ SafeStain (Invitrogen #LC6065). The staining solution was discarded, and the gel was washed three times with 100 mL MilliQ water for 1 hour each, upon which a visible band representing the loaded proteins was clearly present. The visible region of the stained band was excised with a clean scalpel and transferred to a microtube. For experimental replicates and separate experimental condition, separate replicates were loaded with at least 1 empty lane in between to ensure that a gel slice could be obtained that was uncontaminated with other samples.

Gel slices were washed with water, 50% acetonitrile/50% water, acetonitrile, ammonium bicarbonate (100 mM), followed by 50% acetonitrile/50% ammonium bicarbonate (100 mM). The solution was removed, and the gel slices were dried in a speed vac. The gel slices were reduced with dithiothreitol (10 mM in 100 mM ammonium bicarbonate) to 56° C. for 45 min. The solution was removed and discarded. The gel slices were alkylated with 2-chloroacetamide (55 mM in 100 mM ammonium bicarbonate) and incubated in the dark at ambient temperature for 30 min. The solution was removed and discarded. The gel slices were washed with ammonium bicarbonate (100 mM) for 10 min on a shaker and an equal amount of acetonitrile was added and continued to wash for 10 min on a shaker. The solution was removed, discarded and the gel slices were dried in a speed vac for 45 min. The gel slices were cooled on ice and a cold solution of trypsin (Promega, Madison, WI) 12.5 ng/μL, in ammonium bicarbonate (100 mM) was added, enough to cover the gel slice. After 45 min, the trypsin solution was removed, discarded and an equal amount of ammonium bicarbonate (50 mM) was added and incubated overnight at 37° C. while mixing. Samples were spun down in a microfuge and the supernatants were collected. Peptides were extracted from the gel slices by adding 0.1% trifluoroacetic acid (TFA) enough to cover the slices and mixed at ambient temperature for 30 min. An equal amount of acetonitrile was added, and the samples were mixed for an additional 30 min. The samples were spun on a microfuge and the supernatants were pooled. The extract was dried using a speed vac. Samples were desalted using ZipTip C18 (Millipore, Billerica, MA) and eluted with 70% acetonitrile/0.1% TFA. The desalted material was taken to dryness in a speed vac.

Sample preparation of magnetic bead on-bead digests for LC/MS. HCC70 3D spheroid cultures were treated with azidohomoalanine, labeled with DBCO-sulfo-biotin, and subjected to lysis and streptavidin magnetic bead immobilization, as described above. Magnetic bead samples were washed four times with 8 M urea in 50 mM ammonium bicarbonate. Cysteines were reduced with tris(2-carboxyethyl) phosphine (10 mM final) at room temperature while mixing at 1300 rpm for 20 min followed by alkylation with 2-chloroacetamide (5 mM final) while mixing at 1300 rpm at room temperature in the dark for 20 min. Proteins were digested with 500 ng Lys-C/Trypsin (Promega) a 37° C. for 4 hr while shaking at 1300 rpm. The urea concentration was reduced to 1 M and samples continued mixing at 1300 rpm at 37° C. overnight. The next day, the samples were removed from the heat block, cooled to room temperature, and acidified with formic acid (5% final). Samples were mixed at 1300 rpm for 5 min. The samples were placed on a magnetic rack and the supernatant was transferred to another tube. The beads were washed with 2% acetonitrile/0.1% formic acid and combined with the supernatant. The supernatants were concentrated in a speed vac. Samples were desalted using Harvard C18 (Harvard Apparatus, Holliston, MA) and eluted with 70% acetonitrile/0.1% TFA. The desalted material was taken to dryness in a speed vac. To remove detergent, samples were desalted over TopTips (TT10HEA, PolyLC, Columbia, MD). The TopTip was washed and equilibrated with 85% acetonitrile/15 mM ammonium formate, pH 2.8. Samples were taken up in 85% acetonitrile/15 mM ammonium formate, pH 2.8 and loaded onto the tips. The samples were washed with 85% acetonitrile/15 mM ammonium formate, pH 2.8 and eluted with 15 mM ammonium formate pH 2.8. Samples were concentrated in a speedvac.

Orbitrap fusion LCMS. Desalted samples were brought up in 2% acetonitrile in 0.1% formic acid and were analyzed by LC/ESI MS/MS with a Thermo Scientific Easy-nLC 1000 (Thermo Scientific, Waltham, MA) nano HPLC system coupled to a tribrid Orbitrap Fusion (Thermo Scientific, Waltham, MA) mass spectrometer. In-line de-salting was accomplished using a reversed-phase trap column (100 m×20 mm) packed with Magic C18AQ (5-μm 200A resin; Michrom Bioresources, Bruker, Billerica, MA) followed by peptide separations on a reversed-phase column (75 m×270 mm) packed with Magic C18AQ (5-μm 100A resin; Michrom Bioresources, Bruker, Billerica, MA) directly mounted on the electrospray ion source. A 90-minute gradient from 5% to 28% acetonitrile in 0.1% formic acid at a flow rate of three hundred nL/minute was used for chromatographic separations. The heated capillary temperature was set to 300° C. and a static spray voltage of 2200 V was applied to the electrospray tip. The Orbitrap Fusion instrument was operated in the data-dependent mode, switching automatically between MS survey scans in the Orbitrap (AGC target value 500,000, resolution 120,000, and maximum injection time fifty milliseconds) with MS/MS spectra acquisition in the linear ion trap using quadrupole isolation. A three second cycle time was selected between master full scans in the Fourier-transform (FT) and the ions selected for fragmentation in the HCD cell by higher-energy collisional dissociation with a normalized collision energy of 27%. Selected ions were dynamically excluded for 20 seconds and exclusion mass by mass width+/−10 ppm.

Label-free quantification data analysis of LC/MS samples. Data analysis was performed using Proteome Discoverer 2.5 (Thermo Scientific, San Jose, CA). The data were searched against Uniprot Human (Uniprot UP000005640 Mar. 7, 2021) and cRAP (http://www.thegpm.org/crap/) fasta files. In the specific case in which the abundance of EHS matrix proteins was evaluated, a search was also performed against the Uniprot Mus musculus files (however, due to sequence homology the same conclusions were drawn regardless of whether the Mus musculus database was included in the search). Trypsin was set as the enzyme with maximum missed cleavages set to 2. The precursor ion tolerance was set to 10 ppm and the fragment ion tolerance was set to 0.6 Da. Variable modifications included oxidation on methionine (+15.995 Da), DBCO-PEG4-desthiobiotin AHA on methionine (+712.387 Da), DBCO-sulfo-biotin AHA on methionine (+648.211 Da), and methionine conversion to AHA (−4.986 Da). Dynamic modifications on the protein N-terminus included acetylation (+42.011 Da), methylation (+14.016 Da) and methionine loss plus acetylation (−89.030 Da). Static modifications included carbamidomethyl on cysteine (+57.021 Da). Data were searched using Sequest HT. All search results were run through Percolator for scoring and identified peptides were filtered for 1% peptide-level false discovery rate using q value of 0.01. LFQ analysis using Minora was performed.

### Example 2

Estrogen receptor positive breast cancer is the most common type of breast cancer, representing around two-thirds of breast cancer cases. While targeted therapies that disrupt estrogen signaling (i.e., endocrine therapies) have significantly improved patient outcomes, acquired therapy resistance remains a persistent challenge. Among breast cancer patients who initially exhibit complete response to therapy, around 20-30% will relapse with recurrent disease within 15 years. In the metastatic setting, ER+ breast cancer tumor invariably develops resistance to endocrine therapies. While significant work has been performed attempting to understand the genetic mechanisms of endocrine therapy resistance, much less is known regarding the role of intercellular signaling in ER+ breast cancer therapy resistance. Estrogen signaling may induce intercellular signaling factors that support proliferation, and these factors may also be involved in mediating resistance to therapy. As such, technologies that identify novel proteins involved in intercellular communication of ER+ breast cancer may result in the discovery of new therapeutic targets.

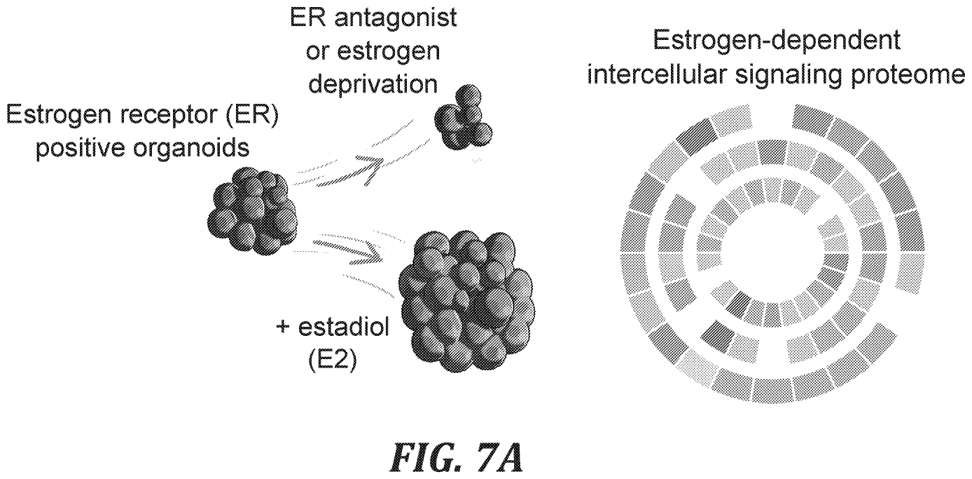

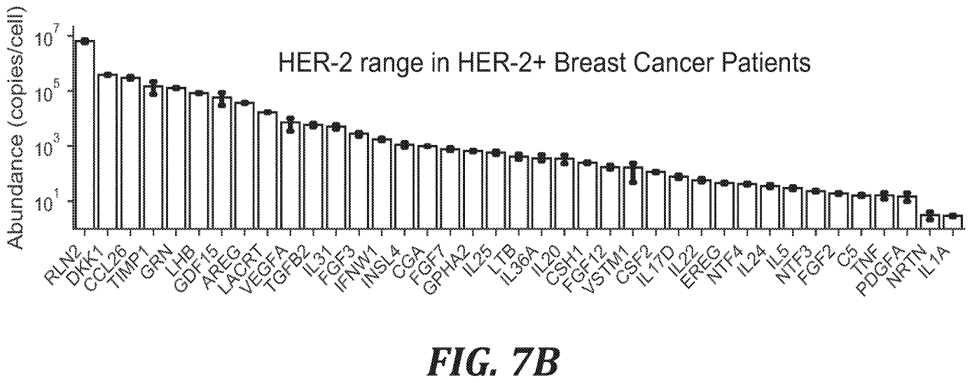

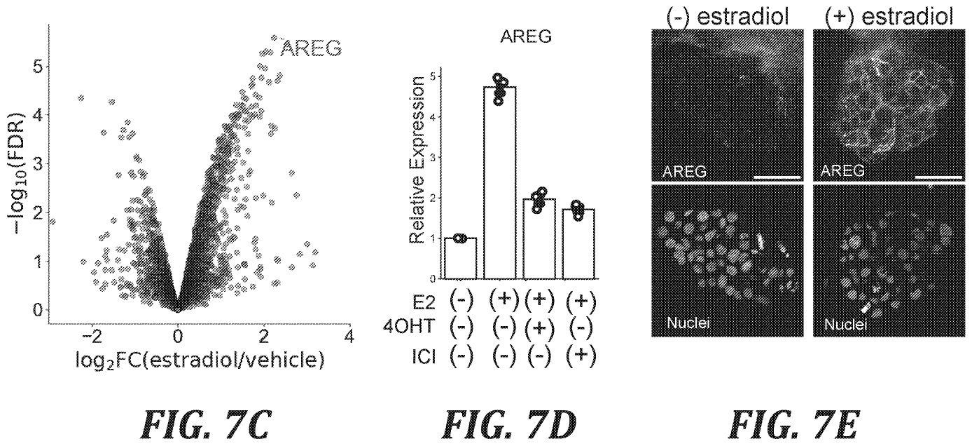

The present method enables the discovery of supraphysiological concentrations of intercellular signaling proteins in ER+ breast cancer. To evaluate the role of intercellular signaling factors in ER+ breast cancer, the disclosed method was used to discover and quantify intercellular signaling factors in the MCF7:WS8 cell line. MCF7:WS8 is an aggressive subline of the MCF7 cell line that exhibits high sensitivity to estrogen, rendering it an excellent model system for examining the role of intercellular signaling in estrogen-dependent proliferation. The aim was to identify (1) how the intercellular signaling proteome of MCF7:WS8 cells changes in response to estrogen (estradiol) and to therapies that disrupt estrogen signaling (e.g., tamoxifen or fulvestrant) (FIG. 7A).

Olink's NPX relative abundance values were converted to estimates of the absolute abundance of each protein in the array of each enriched pericellular fraction (FIG. 7B). Prior published measurements of the range of HER-2 proteins levels in HER-2 positive patients were used to place the absolute abundances in context. See Onsum et al., Am. J Pathol. 183:1446-1460, 2013. Treatment with HER-2 binding antibodies (e.g., Herceptin) is widely used in the clinic in patients with HER-2 positive breast cancer, and thus, it serves as useful benchmark for evaluating the abundance of candidate proteins that may serve as targets in therapies. Proteins in the PEA 3072-plex panel bearing Uniprot annotations of “growth factor activity,” “cytokine activity,” or “hormone activity” were focused on to identify highly abundant intercellular factors that may be relevant to intercellular communication.

Surprisingly, several proteins known to have either growth factor, cytokine, or hormone activity were present in the intercellular spaces of estradiol-treated MCF7:WS8 cells at abundances that matched or exceeded the HER-2 benchmark. Among proteins that were quantified at abundances lower than the HER-2 benchmark, several were still found at supraphysiological concentrations orders of magnitude greater than their known active concentrations. For example, recombinant amphiregulin (AREG) has been shown to exhibit activity in 2D culture of breast epithelial MCF10A cells at concentrations around 5-50 ng/mL when added as an exogenous media supplement. See Berquin et al., Oncogene 20:4019-28, 2001. However, in these experiments, AREG was found to exist in pericellular spaces of MCF7:WS8 spheroids at a concentration in the eluted solution of 0.7 fg/cell. Accounting for previous measurements of the volume of intercellular spaces per cell in breast cancer organoids, 0.152 μL (see Wrenn et al., Cell 183:395-410, 2020), this abundance/cell implies an intercellular concentration of AREG in MCF7:WS8 spheroids of about 46,000 ng/mL. Thus, the disclosed method shows that AREG exists at an intercellular concentration in this specific ER+ breast cancer model that is approximately 1,000 times higher than its known active concentration range.

Use of the present method in ER+ breast cancer reveals that AREG is induced by estradiol and accumulates in intercellular spaces. The next aim was (1) to determine which of these intercellular proteins were specifically induced by estradiol, and (2) to identify proteins that may be particularly relevant to estrogen-dependent proliferation. In response to estradiol (FDR<0.05), at least 188 proteins were upregulated by at least 2-fold compared to estrogen-deprived controls. Among these, AREG was the most upregulated protein in response to estradiol (FDR=0.003, t-statistic 16.2, FIGS. 7C and 7D). To independently confirm that AREG was localized to intercellular spaces in MCF7:WS8 spheroids, immunofluorescence and confocal microscopy were performed using a monoclonal antibody targeting AREG (FIG. 7E). AREG was localized to intercellular spaces, as it co-localized with the cortical F-actin (as visualized by phalloidin staining). Importantly, the magnitude of the fluorescence intensity was significantly increased in estradiol-treated MCF7:WS8 spheroids, consistent with the PEA-results which showed strong induction by estradiol. Taken together, AREG may play a significant role in mediating estrogen-dependent growth.

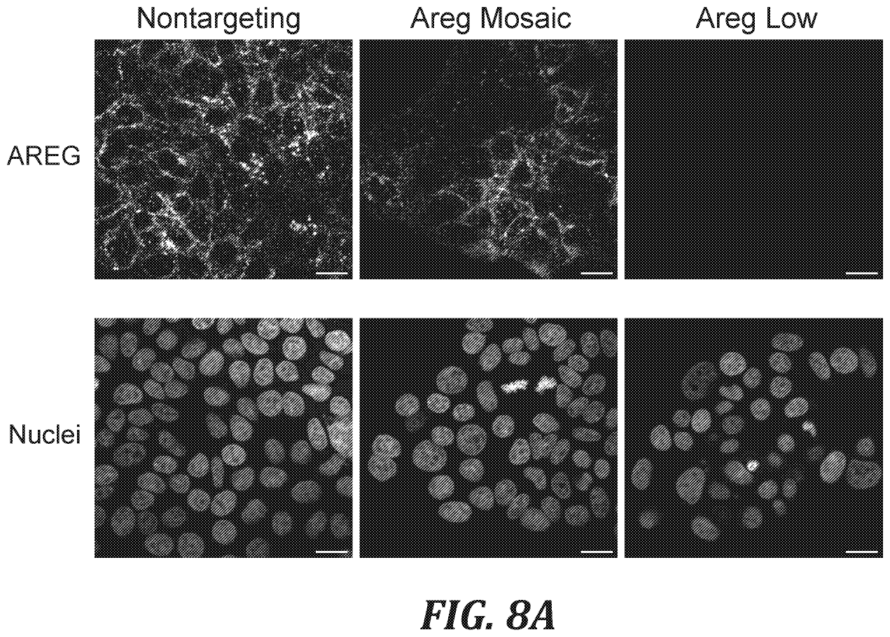

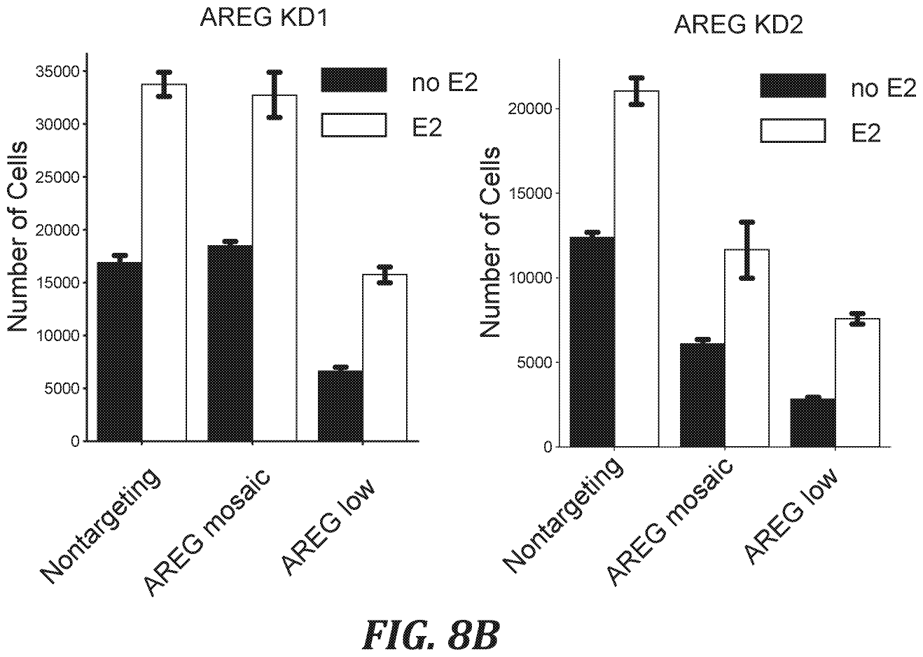

AREG is essential for prolferation of ER+ breast cancer, mediates its effects through intercellular signaling, and promotes resistance to endocrine therapy. The next aim was to evaluate the role of AREG in estradiol-dependent proliferation. MCF7:WS8 cells were transduced with lentiviral vectors expressing two different AREG-targeting shRNAs, each with a GFP reporter. The shRNA knockdowns maintained a heterogeneous population of AREG high (GFP low) and AREG low (GFP high) cells throughout puromycin selection (FIG. 8A). The expectation was that AREG high (GFP low) cells rescued their AREG low (GFP high) neighbors through AREG-mediated intercellular communication, allowing the cells to be stably maintained despite an intrinsic growth defect.

Thus, fluorescence-activated cell sorting (FACS) was performed to purify the AREG low (GFP-high) population and compare its proliferation to the unsorted, “mosaic” population and cells transduced with a non-targeting control shRNA (FIG. 8A). Cells were treated with estradiol or estrogen-depleted media, and the cell proliferation was assessed by genomic-DNA (CyQuant) quantification over 6 days. In both hairpins, the sorted AREG low/GFP high population exhibited lower proliferation than the unsorted and nontargeting control (FIG. 8B). Importantly, the sorted population, which constituted the lower 95% of GFP expression, exhibited no significant difference in proliferation compared to the unsorted population, indicating that this effect was not due to the sorting process. The sorted, AREG low cells exhibited proliferation in the presence of estradiol that was comparable to non-targeting control cells in the estrogen-depleted media, indicating that the effect of AREG knockdown was equal or greater in magnitude compared to the effect of estrogen deprivation. Taken together, these results indicate that AREG induction by estradiol is responsible for estrogen-dependent proliferation, and this effect is achieved through intercellular exchange of AREG.

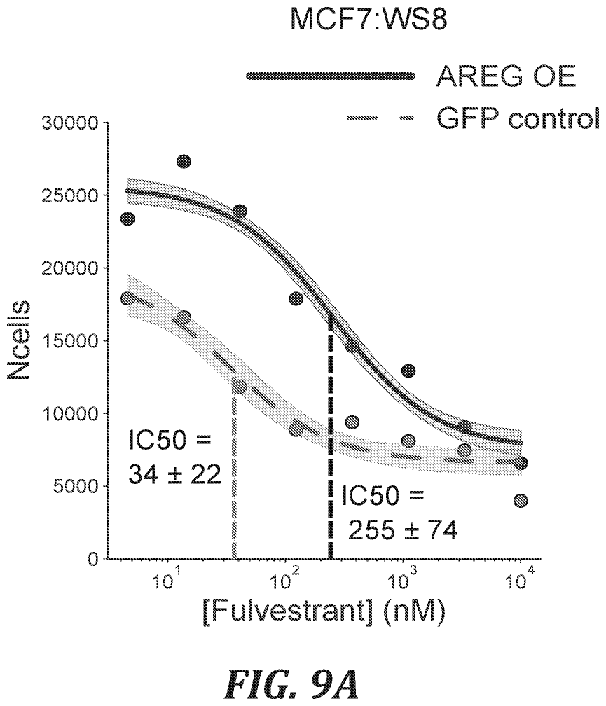

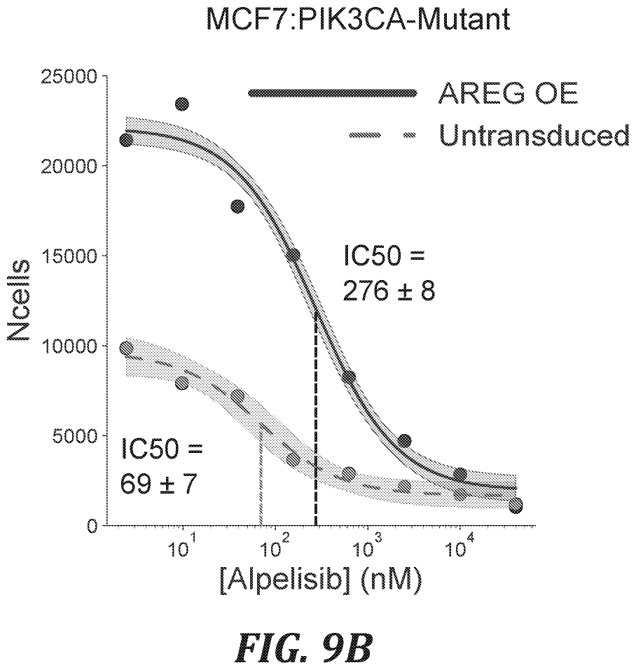

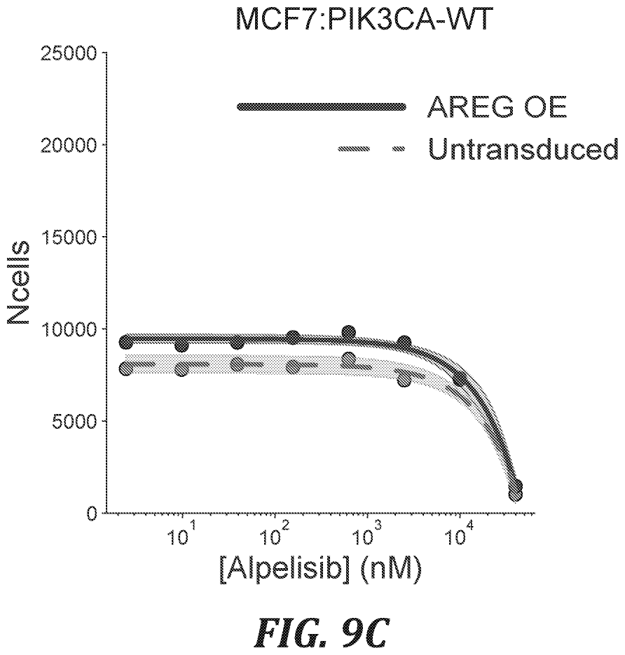

Given that AREG induction by estradiol supports estradiol-dependent proliferation, the expectation was that constitutive overexpression of AREG would confer resistance to drugs that inhibit estrogen signaling (e.g., fulvestrant). Thus, lentiviral transduction was used to generate MCF:WS8 cells that constitutively overexpressed AREG and a GFP reporter, thereby allowing AREG overexpressing cells to be compared to cells overexpressing a biologically inactive open reading frame (ORF) “stuffer” control. The proliferation of each cell line was evaluated in the presence of estradiol (1 nM) and varying concentrations of fulvestrant using a genomic DNA quantification assay (CyQuant). The Hill equation was then used to determine the IC50 of each cell line following fulvestrant treatment (FIG. 9A). AREG-overexpression conferred substantial resistance to fulvestrant treatment, increasing the IC50 from 34 nM (in the control) to 255 nM (in the AREG overexpression line), a 7.5-fold increase in the effective dose required to reduce the proliferation by 50%. These results suggest that increased expression of AREG in ER+ tumors may promote resistance to endocrine therapies. Therefore, targeting AREG signaling may alleviate endocrine therapy resistance.

AREG promotes resistance to PI3K inhibitors in ER+ breast cancer cells harboring PIK3CA mutations. Having shown that AREG can promote resistance to endocrine therapy, the next aim was to investigate additional routes by which AREG may promote resistance to other therapies. Many have attempted to understand therapy resistance from the perspective of genetic mutations, and some genetic mutations have been identified in association with ER+ breast cancer resistance. However, a connection between genetic mutations and intercellular signaling in ER+ breast cancer has not been sufficiently studied. Therefore, genetic mutations associated with ER+ breast cancer therapy resistance may impact intercellular signaling, thereby promoting therapy resistance.

PIK3CA (the gene encoding for phosphoinositide 3-kinase (PI3K)) is the most mutated gene in ER+ breast cancer, occurring in about 40% of cases. Therapies that involve PI3K inhibition have shown clinical benefit in patients with tumors harboring PIK3CA mutations. While PI3K mutation may promote proliferation by constitutive activation of PI3K/AKT signaling, some have shown that PI3K is also involved in cell polarization. This disclosure shows that multicellular organization in ER+ breast cancer, which is heavily influenced by polarization, exerts a pronounced effect on intercellular signaling, as growth factors are concentrated to intercellular spaces at abundances vastly greater than their activity thresholds. Consequently, the expectation was that PI3K mutation may modulate the effects of AREG signaling, and that AREG signaling, in turn, may impact resistance to PI3K inhibitor therapy in a mutation-dependent manner.

The MCF7 cell line harbors two activating E545K mutant PIK3CA alleles. To evaluate the relationship between PIK3CA mutation, AREG expression, and drug resistance, the MCF7 parental line was compared to a previously published isogenic MCF7 line with a wildtype (WT) PIK3CA knock in, which has both mutant alleles corrected (see Beaver et al., Clin. Cancer Res. 19:5413-5422, 2013). MCF7:PIK3CA-WT and the MCF7:PIK3CA-mutant isogenic cell lines were transduced with lentivirus to constitutively overexpress AREG. Then, under estrogen deprived conditions, the growth of MCF7 PIK3CA mutant or MCF7:PIK3CA-WT cells was assessed over 5 days in the presence of varying doses of alpelisib (a PI3K inhibitor) (FIGS. 9B and 9C). Proliferation was quantified by an end-point genomic DNA quantification (CyQuant), and then the concentration-response curve was fit to a Hill equation to extract the IC50 and the asymptotic proliferation in the limit of infinite drug dilution.

In PIK3CA mutant cells, AREG overexpression increased the IC50 4-fold. Additionally, compared to untransduced control cells, PIK3CA mutant cell proliferation increased at least 2-fold over a range of doses. In contrast, alpelisib had no effect on AREG overexpression in MCF7:PIK3CA-WT cells. These results suggest that PIK3CA mutation alters the signaling behavior of AREG, and that in the context of PIK3CA mutation, AREG overexpression confers resistance to PI3K inhibition. Therefore, if these results can be generalized to a broader patient population, they may have important implications for clinical treatment of ER+ breast cancer. For example, if ER+ breast cancer patients with PIK3CA mutations and high AREG expression exhibit resistance to PI3K inhibition, therapies that simultaneously target PI3K and AREG signaling may provide an improved clinical benefit.

The disclosed method led to the discovery of intercellular proteins that are upregulated in response to endocrine therapy. Having established the disclosed method's ability to identify intercellular proteins that are upregulated in response to estradiol and that mediate estrogen-dependent proliferation, the next aim was to investigate what proteins were upregulated in response to drugs that inhibit estrogen signaling (fulvestrant and tamoxifen). Discovering proteins that are upregulated in response to endocrine therapy may yield new therapeutic targets for multiple reasons. For one, proteins that are upregulated in response to endocrine therapy may mediate acute survival in drug-treated cancer cells, and therefore therapies that inhibit the biological activity of such proteins may reduce therapy resistance. Additionally, the discovery of proteins that are specifically upregulated in drug-treated cancer cells, and not healthy tissue, may lead to the development of antibody-drug conjugate therapies that exhibit greater specificity for cancer cells and reduced unwanted off-target effects.

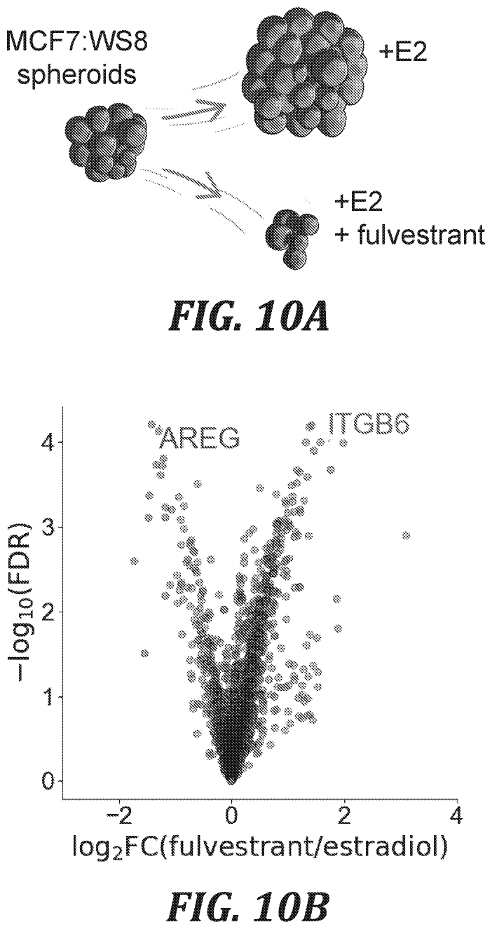

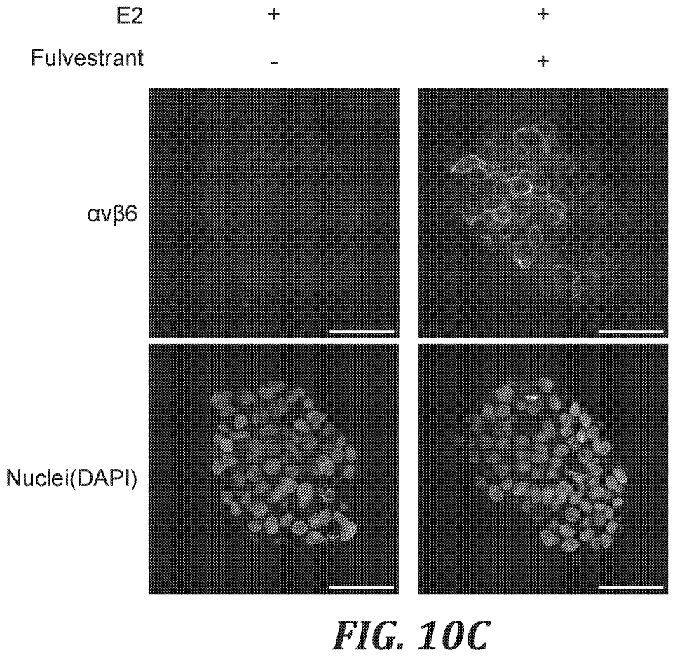

PEA was used to quantify protein abundance in MCF7:WS8 cells treated with estradiol (1 nM) and fulvestrant concomitantly or with estradiol (1 nM) alone. Proteins from cells treated with or without fulvestrant were ranked by a log 2-fold change in abundance and by a false-discovery rate and identified. Several proteins were significantly upregulated in fulvestrant-treated cells (FDR<0.05) (FIG. 10B), with ITGB6 being the most significantly upregulated protein.

Immunofluorescence confocal microscopy was performed on MCF7:WS8 spheroids treated with either estradiol (1 nM) alone or estradiol (1 nM) and fulvestrant (100 nM) concomitantly to confirm that ITGB6 was localized to intercellular spaces in MCF7:WS8 3D spheroids and upregulated in response to fulvestrant (FIG. 10C). ITGB6 is known to form the integrin heterodimer αvβ6, so indirect immunofluorescence was performed using a monoclonal antibody against αvβ6. αvβ6 consistently localized to intercellular spaces, and its fluorescence intensity was higher in fulvestrant-treated cells. ITGB6 is a very promising candidate for antibody-based cancer therapies. αvβ6 is rarely expressed in normal, healthy tissue, so antibody therapies that target αvβ6 are less likely to have unwanted off-target effects in healthy tissue. For indications outside of breast cancer, clinical trials evaluated αvβ6-targeted antibody therapies are already in progress. However, at present, no clinical trials have been initiated specifically evaluating ITGB6 in ER+ breast cancer. These results provide a basis for further investigations to evaluate the therapeutic potential of ITGB6 in ER+ breast cancer. Thus, these results demonstrate that the disclosed method can be used to identify new intercellular proteins that are upregulated in response to ER+ breast cancer therapy, and some of these proteins could be future therapeutic targets.

The disclosed method revealed distinct intercellular signaling responses to tamoxifen treatment between therapy sensitive and therapy resistant ER+ breast cancer cells. Having demonstrated that the intercellular signaling proteome is strongly modulated in acute response to estradiol and endocrine therapies, the next aim was to evaluate how the intercellular signaling proteome changes in cells that develop acquired therapy resistance due to long-term drug therapy. To develop tamoxifen resistant tumor cells, the commonly used tamoxifen-therapy resistant model (TAMR) was employed, wherein mice with MCF7:WS8 xenografts were treated with tamoxifen until tamoxifen resistant tumor cells emerged. Thus, MCF7:WS8 and TAMR represent an isogenic pair derived from the same parental line, and as such provide a useful system for modeling changes that occur during acquisition of therapy resistance.

Three-dimensional cultures of MCF7:WS8 cells and TAMR cells were generated in 2% EHS matrix suspension. Spheroids were treated with either 1) estradiol (10 nM) or 2) estradiol (10 nM) and tamoxifen (100 nM). The spheroids were treated with azidohomoalanine for 24 hours, labeled with DBCO-PEG4-Desthiobiotin, and lysed. Full-length intercellular proteins were enriched by immobilizing desthiobiotin-conjugated proteins on streptavidin magnetic beads, followed by elution in the presence of free biotin. The eluted protein fraction was then subjected to proximity extension assay-based quantification using the Olink® Explore protocol to quantify the abundance of proteins in a 3072-plex panel.

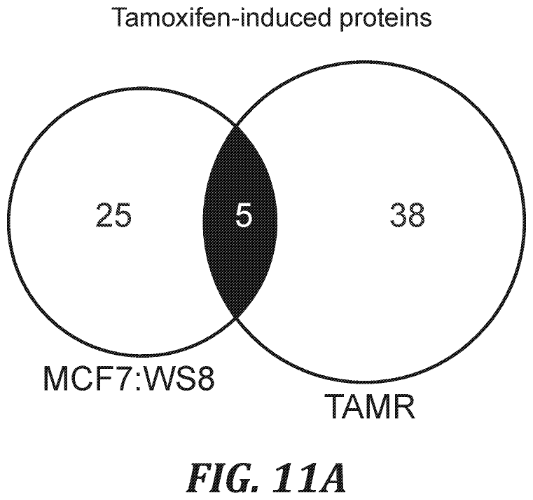

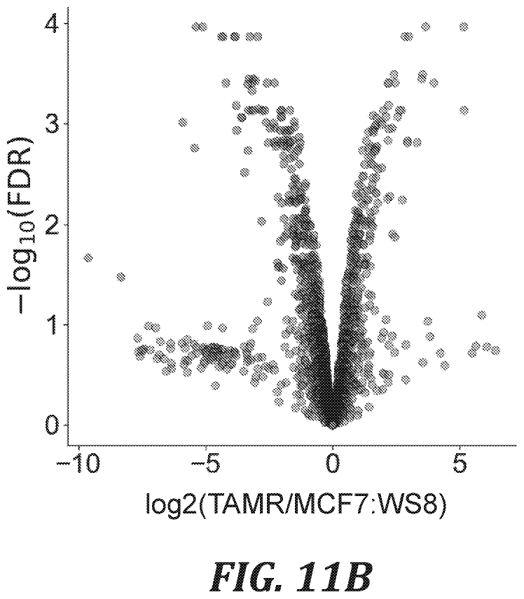

First, a tamoxifen-induced protein signature was developed for each line by comparing how each cell line responded to tamoxifen treatment. Next, the log 2-fold change enrichment was quantified for cells treated with or without tamoxifen. The differential expression matrix was then filtered to identify proteins that were upregulated at least 2-fold in response to tamoxifen (i.e., an FDR<0.05). Thirty proteins were identified that were upregulated in therapy sensitive MCF7:WS8 cells and forty-three proteins that were upregulated in response to therapy resistant TAMR cells (FIG. 11A). Strikingly, only five of these proteins were shared between the two lines. Thus, 88% of the intercellular proteins that are upregulated in response to tamoxifen in therapy resistant cells were found to be upregulated only in therapy resistant cells.

Next, the intercellular protein expression of TAMR cells was compared to all the proteins expressed by MCF7:WS8 cells. This analysis revealed 398 proteins that were differentially regulated (up or down) between TAMR cells and MCF7:WS8 cells at an FDR<0.05 (FIG. 11B). Among these proteins, 108 were upregulated at least 2-fold in TAMR cells (FDR<0.05). Taken together, these results suggest dramatic changes in the intercellular proteome that occur during acquired resistance to tamoxifen therapy. The proteins identified serve as a foundation for identifying candidate proteins that may be targeted to sensitize therapy resistant cells to tamoxifen treatment. More broadly, these results represent a proof-of-principle that the disclosed method can discover biological differences in the intercellular signaling proteome that occur in the context of therapy resistance.

### Example 2 Materials and Methods

Cell lines. MCF7:WS8 and MCF7:TAMR cells were obtained as a gift from Donald McDonnell (Duke University, Durham, North Carolina). The establishment of the MCF7:WS8 line from the MCF-7 parental cell line is described by Pink et al. (Cancer Res. 55:2583-2590, 1995). HEK 293FT cells were purchased from Thermo Fisher Scientific (cat. #R70007). MCF7:TAMR cells were derived from the MCF7:WS8 cell line, and their generation is described by Cocce et al. (Cell Rep. 29:889-903, 2019). HCC70 cells were purchased from ATCC. All cells were cultured in sterile tissue culture incubators maintained at 37° C. and 5% CO₂ under normoxic conditions. Cell lines were routinely tested for mycoplasma and found to be mycoplasma negative.

Antibodies. The following primary antibodies were used for immunofluorescence staining at the indicated dilutions: Rat monoclonal antibody against human Integrin alpha V beta 6, clone avβ6 53a.2, 1:100 dilution (Abcam #ab97588); mouse monoclonal antibody against human amphiregulin, reconstituted at 1 mg/mL in PBS, 1:100 dilution (R&D systems, MAB262-100). Secondary detection was performed using either a goat anti-rat high crossly adsorbed polyclonal secondary antibody, 1:500 dilution (Invitrogen #A11006); or a goat anti-mouse highly cross-adsorbed polyclonal secondary antibody, 1:500 dilution (Invitrogen #A1 1029).

Click chemistry and biotinylation reagents. L-Azidohomoalanine, and DBCO-PEG4-Desthiobiotin were purchased from Click Chemistry Tools (cat. #1066-100, #1108-25, respectively). L-Azidohomoalanine was reconstituted in anhydrous DMSO at 500 mM. DBCO-PEG4-Desthiobiotin was reconstituted in anhydrous DMSO at 300 mM. All labeling reagents were stored at −20° C. under desiccated conditions.

MCF7: WS8 and MCF7: TAMR 2D monolayer culture. Prior to generation of spheroids, MCF7 cells were maintained and propagated in 2D monolayer culture on tissue culture-treated flasks in a tissue-culture incubator at 37° C. and 5% CO₂. Cells were maintained in DMEM/F12 media supplemented with 8% fetal bovine serum, 1% penicillin-streptomycin, 1 mM sodium pyruvate, 2 mM GlutaMax, and MEM non-essential amino acid supplement (1:100 dilution). Media was changed every 2-3 days and cells were passaged upon reaching 70-80% confluence. Cells were passaged by dissociating with TrypLE expression at 37° C., followed by 5:1 dilution with complete cell culture media, centrifugation at 200×g for 3 minutes, and resuspension in complete cell culture media. Cells were propagated following dissociation at a split ratio in the range of 1:4 to 1:6.

Preparation of 2% EHS matrix suspension media. 2% EHS matrix suspension media was obtained by combining ice-cold EHS matrix (Cultrex reduced growth factor basement membrane extract, R&D systems #3433-005-01) with ice-cold complete cell culture media (DMEM-fl2 media containing 8% fetal bovine serum, 1% penicillin-streptomycin, 2 mM GlutaMax, 1:100 MEM non-essential amino acids, and 1 mM sodium pyruvate) at a ratio of 1:49 (for example, 1 mL of EHS matrix at the manufacturer's delivered concentration into 49 mL complete cell culture media). The mixture was immediately mixed well by inverting, and the EHS-supplemented media was then pre-warmed at 37° C. in a water bath to allow the EHS matrix to form crosslinks. A methionine-reduced variation of 2% EHS matrix was prepared as above, except that DMEM/F12 basal media was replaced with methionine-free DMEM/F12. Estrogen depleted media was prepared exactly as above, except that fetal bovine serum was replaced with charcoal stripped fetal bovine serum (Sigma-Aldrich #F6765-500ML), and phenol red-free DMEM/F12 was used.

Generation of MCF7: WS8 and MCF7:TAMR 3D spheroid cultures in 2% EHS matrix. Prior to generation of 3D spheroid cultures, MCF7 cells were passaged twice after revival from liquid nitrogen cryostorage to minimize effects associated with thawing. MCF7 cells were dissociated from 2D TC-treated culture flasks using TrypLE express by incubation for about 5 minutes at 37° C., or until cells fully detached from the surface. The cells were recovered from the flask and diluted 5:1 with complete cell culture media. Cells were pelleted by centrifugation at 200×g for 3 minutes and gently resuspended in phosphate buffered saline (PBS) without calcium or magnesium. Cells were counted with an automated cell counter hemocytometer (Countess). Next, the desired number of cells was pelleted by centrifugation at 200×g for 3 minutes and cells were resuspended in 2% EHS suspension media (prepared according to the recipe above) at a cell density of 150 thousand cells per mL of suspension media. The cell suspension was plated in 6-well ultra-low attachment plates (Corning #3471), and multicellular clusters were allowed to form. Spheroids were dissociated once after 4 days of initial establishment before being plated for experiments.

To dissociate spheroids, spheroids were pelleted by centrifugation at 200×g for 3 minutes and washed twice with PBS (without calcium, magnesium). Cells were then resuspended in Accumax cell dissociation solution (Innovative Cell Technologies #AM105) and incubated in a 37° C. water bath for 5 minutes. The spheroids were triturated with a 5 mL serological pipette ten times. The spheroids were then incubated at room temperature in the dissociation solution, and every 5 minutes the suspension was triturated with a 1 mL micropipette ten times. This process was repeated for up to 30 minutes until at least 80% of the cells were observed to be a single cell suspension when visualized under a phase contrast microscope.

Following dissociation, cells were pelleted at 200×g for 3 minutes and resuspended in PBS (without calcium, magnesium). Cells were counted on a hemocytometer using an automated cell counting system (Countess) and resuspended in 2% EHS matrix suspension media (recipe described above) at a cell density of 150 thousand cells per mL. The cell suspension was plated in 6-well ultra-low attachment plates (Corning #3471), and multicellular clusters were allowed to form.

Estrogen deprivation, azidohomoalanine, and drug treatment of MCF7: WS8 and MCF7:TAMR cells in 2% EHS matrix suspension culture. MCF7 cells were seeded, as described above, in estrogen-depleted 2% EHS matrix suspension culture (containing charcoal-stripped FBS and phenol red free) for 24 hours to create estrogen-starved spheroids. Spheroids were pelleted by centrifugation at 200×g for 3 minutes, then resuspended (at their original spheroid density) in estrogen-depleted 2% EHS matrix culture media containing either 1) No treatment, 2) Beta-estradiol (1 nM), 3) Beta-estradiol (1 nM)+Fulvestrant (100 nM), Beta-estradiol (1 nM)+tamoxifen (100 nM). Cells were cultured for an additional 24 hours, then pelleted by centrifugation and washed three times with PBS (containing calcium, magnesium) to remove excess methionine. The spheroids were then resuspended in methionine-reduced, estrogen-depleted 2% EHS matrix media containing azidohomoalanine (500 pM) and one of the above drug treatments. Spheroids were cultured for an additional 24 hours before proceeding to DBCO-PEG4-Desthiobiotin labeling.

DBCO-PEG4-Desthiobiotin and DBCO-sulfo-Biotin labeling in 2% EHS matrix suspension culture. After 24 hours of azidohomoalanine labeling, spheroids were washed three times with PBS (containing calcium and magnesium) to remove unincorporated azidohomoalanine by centrifugation at 200×g for 1 minute, aspiration of the supernatant, and resuspension in the PBS wash buffer. The spheroids were then resuspended in a PBS solution containing 1% bovine serum albumin and DBCO-PEG4-Desthiobiotin at a concentration of three hundred micromolar. The cells were incubated at 37° C. in 6-well ultra-low-adhesion plates for 30 minutes in a tissue culture incubator maintained at 5% CO₂ to allow the SPAAC reaction to proceed. The excess unreacted reagent was then removed by washing the cells four times with PBS (containing calcium and magnesium) by repeated centrifugation at 4° C. (200×g, 1 minute) and resuspension. Samples were then pelleted by centrifugation at 200×g for 3 minutes, the supernatant was removed, and pellets were snap-frozen in liquid nitrogen by submerging the tube for 5 minutes. Snap-frozen pellets were stored at −80° C. until ready for use.

Preparation of DBCO-PEG4-Desthiobiotin labeled spheroid lysates. Lysis was performed exactly as described in Example 1. Prior to thawing spheroid samples, a lysis buffer consisting of 0.1% SDS, 1% Triton X-100, 150 mM NaCl, 20 mM Tris, 1 mM EDTA, pH 7.4 was freshly prepared and supplemented with protease inhibitors (Halt Protease inhibitor cocktail, 100× dilution, Thermo Fisher Scientific #87785) and phosphatase inhibitors (Halt Phosphatase inhibitor cocktail, 100× dilution, Thermo Scientific #78420). Spheroid pellets were thawed on ice in direct contact with ice-cold lysis buffer (one hundred microliters of lysis buffer per one million cells seeded for the spheroids). The mixture was periodically vortexed to facilitate thawing until the pellet was fully thawed. Once the pellets were fully thawed, samples were briefly vortexed to fully resuspend the cells, and samples were sonicated using a probe sonicator with six pulses, 0.5 seconds each. Samples were then incubated with gentle orbital agitation at 4° C. for 1 hour. After incubation, samples were again sonicated using a probe sonicator with six pulses, 0.5 seconds each. Next, samples were centrifuged in a pre-chilled microcentrifuge at 4° C. at 12,000×g for 10 minutes to remove insoluble material. The supernatant (representing the cleared spheroid lysate) was collected and used for the next step of the enrichment.

Streptavidin magnetic bead enrichment. Streptavidin magnetic bead enrichment of DBCO-PEG4-Desthiobiotin labeled cells was performed as follows. One micron-sized magnetic polystyrene beads covalently functionalized with streptavidin (binding potential: 2,500 pmoles free biotin/mg beads), bearing a hydrophilic, carboxylated surface (Streptavidin MyOne C1, Thermo Fisher cat #65001), and a magnetic separation rack (DynaMag 2, Thermo Fisher Scientific cat #12321D) were used to enrich proteins that had been covalently labeled with either a biotin or desthiobiotin enrichment handle. For every 100 μL of sample, 200 μL of the 10 mg/mL magnetic bead suspension were used. Prior to use, the beads were washed 3 times with a tris-buffered saline wash buffer consisting of 150 mM NaCl, 20 mM Tris, 0.2% Tween-20, pH 7.6 (from here, referred to as “TBST”) by separating the beads on the magnetic stand, removing the supernatant, and resuspending the beads in 500 microliters of TBST. Next a magnetic bead pellet was prepared by separating the beads on a magnetic stand and removing the supernatant. The cleared spheroid lysate was added directly to the magnetic bead pellet, and the beads were resuspended in the lysate by dragging the tube along a microtube rack repeatedly and relying on the vibrations generated by the uneven microtube rack surface (this method of dragging the tube along the microtube rack was found to be superior to vortexing). The bead/lysate mixture was then incubated for 3 hours at room temperature with orbital agitation to prevent the beads from settling. Next, the magnetic beads were separated on the magnetic stand, and the supernatant was collected and kept as the “unbound fraction.” The beads were then washed three times with TBST by repeatedly resuspending the pellet (by running the tube along a microtube rack) and then separating the magnetic beads. No incubation time was applied during the washes.

To elute bound proteins from the beads, an elution buffer containing 0.1% SDS, 1% Triton X-100, 150 mM NaCl, 20 mM Tris, 1 mM EDTA, 3 mg/mL biotin was prepared. Biotin free acid is only soluble in water in its deprotonated form, so a 2× concentrated form of the buffer was initially prepared and titrated with sodium hydroxide until all biotin fully dissolved and the pH reached 7.4-7.6. The buffer was then diluted with MilliQ water to reach the composition. 100 μL of elution buffer were used per 100 μL of starting lysate material. The pelleted beads, containing the immobilized proteins, were resuspended in the elution buffer by dragging the tube along a microtube holder. The samples were first incubated for 2 hours at 37° C. while shaking at 200 RPM, followed by a 16-hour incubation at 4° C., with gentle orbital agitation. Next, the magnetic beads were separated on a magnetic stand, and the supernatant was collected as the final elution fraction. The eluted fraction was snap-frozen by submerging the tube in liquid nitrogen for 5 minutes, and the sample was stored at −80° C. until ready for analysis by the proximity extension assay.

Intercellular protein discovery by the proximity extension assay and NPX normalized protein abundance calculation. Proteins were measured using the Olink® Explore 3072 panel (Olink Proteomics AB, Uppsala, Sweden) according to the manufacturer's instructions. For each experimental replicate, eighty microliters of elution solution (representing labeled proteins from eight hundred thousand cells), was used directly for the Olink Explore protocol. The Olink Explore 3072 panel uses a 3072-plex panel of oligonucleotide-conjugated antibody probe pairs. Each antibody probe pair is designed to bind to the target, resulting in hybridization of the complementary oligonucleotides provided by each member of the paper. DNA polymerase is then used to extend only oligonucleotides that have hybridized, producing a unique DNA barcode each time a protein is bound to both members of an antibody pair whose conjugated oligonucleotides were designed to hybridize. Library preparation was then performed to add sample identification indices and the necessary nucleotides for illumine sequencing. Libraries were subjected to a bead-based purification and quality was assessed using an Agilent 2100 bioanalyzer (Agilent Technologies, Palo Alto, CA). Samples were then subjected to next-generation sequencing using the Illumina NovaSeq 6000. The data was then quality controlled and converted into Normalized Protein eXpression (NPX) values, which represent Olink's unit of relative abundance. For quality control, three internal controls were spiked into each sample and used to evaluate the performance of 3 steps in the protocol: an incubation control with a non-human antigen and its matching antibody probes, an extension control consisting of an IgG antibody conjugated to a pair of hybridizing oligonucleotides, and an amplification control consisting of a complete double-stranded DNA amplicon. The Olink Explore protocol was also performed on a set of external controls: two different pooled human plasma samples, three negative controls, and three plate controls. Each plate control is a plasma sample that is used to normalize across plates.

The NPX value was calculated by first computing ExtNPX(assay I, sample J), according to ExtNPX(assay i, sample j)=log 2(counts(assay i, sample j)/(counts(Extension control, sample j)); which normalized the counts of each sample to the counts of a known standard. Next the NPX value was computed as NPX(assay i, sample j)=ExtNPX(assay i, sample j)−median (ExtNPX(PC, assay i)); which normalized across plates. The limit of detection for each sample was defined to be equal to an NPX value that is three standard deviations above the median of the negative control samples. All proteins below the LOD were excluded from the list of discovered proteins. All assay validation data (detection limits, intra- and inter-assay precision data, predefined values, etc.) are available on manufacturer's website.

Calculation of absolute protein abundance from Olink NPX relative abundance. The Olink NPX value provides a measure of relative abundance in which differences in NPX values between two samples within a given protein correspond to log 2-fold changes in protein abundance. The NPX value is not a measure of absolute abundance. To obtain estimates of absolute abundance from the NPX value, the following procedure was developed, which utilizes Olink's published calibration data and absolute protein detection lower and upper limits, together with the NPX values of controls, to normalize the NPX values against the calibration data. This analysis was performed using custom scripts written in Python.

Olink's calibration data provides the relationship between absolute protein abundance and NPX value for the calibration experiment. While the linear dynamic range of the assay is expected to behave similarly across similar samples, the NPX values that correspond to a given absolute concentration may be arbitrary shifted by an experiment-specific normalization factor. As such, let log 2(c)=f(NPXc) denote the function that takes the NPX values in the calibration dataset, NPXC and converts them to concentration in pg/mL. Let log 2(c)=g(NPX) denote the function that maps NPX values in the experimental dataset onto absolute concentrations. Within a given experiment, the NPX values of a given protein for two samples i, j, NPXi, NPXj satisfy the relationship:

$$NPXi - NPXj = log 2 ( ci / cj ) = log ( c i ) - log ( c j ) = g ( NPXi ) - g ( NPXj )$$

where ci and cj are the concentrations of the protein in sample i and sample j. Since this relationship holds for any pair of values NPXi, NPXj, it follows that the following expression is a constant:

$$( NPXi - NPXj ) / g ( NPXi ) - g ( NPXj ) ) .$$

The same also holds for f(NPXc). Moreover, since differences in NPX values consistently correspond to fold-changes in concentration, the corresponding expressions for g(NPX) and f(NPX) must equal the same constant. Then, g(NPX) and f(NPX) are linear functions with the same slope that differ by an arbitrary shift along the NPX axis. The concentration mappings then can be expressed with respect to the limit-of-detection in the calibration dataset, LOD, and corresponding lower limit of quantification in concentration units (LLOQ) as:

$$f ( NPXc ) = m * [ NPX - L O {D}_{c} ] + log 2 ( LLOQ )$$

$$g ( NPX ) = m * [ ( N P X - NPX 0 ) - L O {D}_{c} ] + log 2 ( LLOQ )$$

where NPXO is a normalization constant. The slope, m, is provided by Olink's calibration data, as is LLOQ. In general, because LOD is NPX units, LOD is the experimental dataset is not equal to LOD. However, the mappings f and g, must produce the same value (specifically, log 2(LLOQ)) when evaluated at their respective LOD. Then, it follows that:

$$LOD - NPX 0 - {LOD}_{c} = 0$$

$$NPX 0 = LOD - L O {D}_{c} .$$

In other words, the calibration curve for the experimental dataset is obtained by shifting the experimental calibration curve along the NPX axis by a constant value equal to the difference in LOD values between the experimental and calibration data sets. Thus, the log 2-transformed concentrations values were obtained directly from the NPX value, the experimental LOD, the calibration LODc, the calibration LLOQ, and the calibration slope m according to:

$$log 2 ( c ) = m * [ NPX - L O D ) + log 2 ( LLOQ ) .$$

Differential expression analysis of PEA quantification data. Differential expression analysis to identify statistical differences in protein expression between treatment conditions was performed using the limma (v. 3.50.3) and edgeR (v.3.36.0) packages in R (v. 4.1.1). Specifically, the matrix of NPX values was fitted to a linear model of the form y~Treatment using limma. The linear fit coefficients were subjected to empirical Bayes smoothing using the eBayes function in edgeR. Contrasts in the linear modeling coefficients were then fitted using limma and subjected to empirical Bayes smoothing to obtain the log 2-fold change, t-statistic, and unadjusted p-value. The p-values were then corrected for multiple comparisons using the Benjamini and Hochberg false-discovery rate correction procedure to obtain the final FDR.

Indirect immunofluorescence staining of pericellular or cytosolic control proteins. MCF7:WS8 spheroids were prepared in 2% EHS suspension culture (following one passage in 3D culture) in estrogen-depleted, phenol red-free media with charcoal-stripped fetal bovine serum (FBS). 24 hours after plating, media was replaced with estrogen-depleted media that was supplemented with either 1) no treatment, 2) estradiol (1 nM), 3) estradiol (1 nM)+fulvestrant (100 nM), and spheroids were cultured for 48 hours in the presence of drug. 48 hours after drug treatment, spheroids were pelleted by centrifugation at 200×g for 3 minutes and resuspended in a fixation buffer of 4% paraformaldehyde (PFA) in PBS (with calcium and magnesium). Spheroids were incubated in fixation solution for 10 minutes at room temperature in 24-well plates with No. 1.5 coverglass bottoms. After fixation, spheroids were allowed to settle by gravity, the fixative was aspirated, and spheroids were washed once with PBS containing 200 mM glycine. After settling by gravity and resuspension, spheroids were resuspended in the PBS/glycine quenching buffer and incubated for 20 minutes at room temperature with gentle agitation. The spheroids were then washed four times with PBS (with calcium, magnesium) by resuspension in the wash buffer, incubation for 10 minutes, gravity settling, and aspiration of the wash buffer.

Spheroids were permeabilized for 1 hour with PBST (PBS, 0.1% Tween-20, with calcium, magnesium), then blocked for 1 hour with PBST containing 10% normal goat serum. Primary antibody labeling solution was prepared by diluting primary antibodies 1:100 in a buffer of PBS, 0.1% (v/v) Tween-20, 0.1% (w/v) BSA. Spheroids were allowed to settle by gravity and resuspended in antibody labeling buffer, then incubated for 16-20 hours at 4° C. with gentle orbital agitation. Samples were washed four times, 10 minutes each, with PBST by gravity settling and resuspension. Secondary antibody labeling solution was prepared by diluting secondary antibodies 1:500 in a buffer of PBS, 0.1% (v/v) Tween-20, 0.1% (w/v) BSA. Samples were incubated in secondary antibody labeling buffer for 16-20 hours at 4° C. with gentle orbital agitation. Spheroids were washed four times with PBST, 10 minutes each, then counterstained with DAPI and phalloidin-alexafluor568 in PBST for 1 hour. Samples were washed twice with PBST and twice with PBS, 10 minutes each. Spheroids were maintained in PBS at 4° C. and were used within 4 days for imaging by confocal fluorescence microscopy.

Confocal microscopy. Samples were plated into 24-well plates containing a No. 1.5 polymer coverglass. Confocal mages were acquired using an Andor CSU-W confocal spinning disk on a Leica DMi8 inverted microscope using a 40X water immersion objective.

Plasmid preparation for lentiviral vectors. All plasmids were purchased in the form of bacterial glycerol stocks. Glycerol stocks were streaked onto agar plates containing one hundred pg/mL ampicillin and incubated for 16 hours. An isolated colony was selected from the plate and cultured in a 5 mL Luria-Bertani (LB) starter culture containing one hundred pg/mL ampicillin. After 8 hours, a portion of the starter culture was diluted 1:500 into 100 mL LB media one hundred pg/mL ampicillin for inoculation. Culture was performed while shaking at 250 RPM at 37° C. for 16 hours. After 16 hours, bacterial pellets were harvested by centrifugation at 4,000×g for 10 minutes at 4° C., and plasmids were purified using a Qiagen Highspeed Plasmid Midi Kit (Qiagen #12643) according to the manufacturer's protocol. Plasmids were resuspended in ultra-pure nuclease-free water and their concentration and purity was determined spectrophotometrically using a NanoDrop.

Lentiviral particle generation. Lentiviral particles were generated by transfection of HEK-293FT cells. HEK-293FT cells were cultured in high glucose DMEM with 10% FBS, GlutaMax 1% penicillin-streptomycin, and 500 μg/mL Genetecin. HEK-293FT cells were passaged twice prior to transfection and allowed to reach 80% confluence. Upon reaching 80% confluence, cells were co-transfected with lentiviral vectors and packaging plasmids (3 μg MD2.G, eight pg PS Pax, twelve pg lentiviral vector containing the plasmid of interest). Lipoplexes were formed containing the three plasmids by combining with Lipofectamine 3000 (Thermo Fisher Scientific #L3000015) according to the manufacturer's instructions. The media on HEK-293FT cells was replaced with serum-free Opti-MEM media (Gibco #31985062), and the lipoplex solution was added dropwise. Cells were incubated in contact with the lipoplex solution for 16 hours at 37° C., 5% CO₂.

After 16 hours, the transfection media was removed and replaced with complete cell culture media (minus the Genetecin). 24 hours after replacing the media, the supernatant (containing lentiviral particles) was collected. The supernatant was cleared of cellular debris by centrifugation at 2,000×g for 5 minutes, and the supernatant was collected and filtered through a 0.45-micron PVDF filter membrane. The lentiviral particles were concentrated 100-fold using the Lenti-X concentrator system according to the manufacturer's protocol (Takara Bio #631232). Concentrated lentiviral particles were then flash frozen by submerging the tube in liquid nitrogen and stored at −80° C. until ready for use.

Lentiviral transduction. Prior to lentiviral transduction, a functional titer was performed (using the transduction protocol below) by transducing MCF7 cells with serial dilutions of lentiviral particles. Prior to transduction, MCF7 cells were plated on tissue-culture treated flasks at a cell density of 25,000 cells/cm². 24 hours after plating, the media was replaced with transduction media consisting of serum-free DMEM/F-12 with GlutaMax (1:100) and sodium pyruvate (1:100) supplemented with eight ug/mL protamine sulfate (1:500 dilution of 4 mg/mL stock). An appropriate volume of lentiviral particles representing a multiplicity of infection (MOI) of one was diluted into one hundred uL of PBS. The lentiviral particle dilution was then added dropwise to the media in the MCF7:WS8 cell culture, and the flask was mixed by gently swirling by hand. Cells were then incubated in contact with lentiviral particles for 24 hours at 37° Celsius, 5% CO₂.

Twenty-four hours after adding lentiviral particles, the media was exchanged with complete cell culture media. Cells were cultured for an additional 48 hours before adding selection antibiotic. After 48 hours of culture in complete culture media, the media was exchanged with selection media, consisting of complete cell culture media containing two pg/mL puromycin. MCF7 cells were maintained in selection media and passaged upon reaching 80% confluence for 2 weeks prior to use in experiments. An untransduced control was subjected to the exact same culture conditions, and it was visually confirmed by phase contrast microscopy that all cells in the untransduced control had died in selection media.

ShRNA Lentiviral Plasmid Sequences. Lentiviral shRNA vectors were obtained from the Dharmacon GIPZ Lentiviral shRNA library. The specific vectors used were obtained directly from a shared shRNA database provided to Fred Hutchinson Cancer Center researchers by the Fred Hutch Genomics Services core. Each vector consists of microRNA adapted shRNA that is contained within the 3′ UTR of a polycistronic transcript consisting of an internal ribosomal entry site flanked by a turboGFP reporter and puromycin resistance gene. The sequences of the shRNA shown in SEQ ID NO:4 (AREG KD1) and SEQ ID NO:5 (AREG KD2).

AREG overexpression plasmid vector and ORF stuffer control. Customer overexpression vectors were obtained from VectorBuilder. The plasmid is built on a pLV lentiviral plasmid backbone with a dual-promoter expression cassette. The dual-promoter expression cassette consists of a hCMV promoter driving expression of an eGFP promoter and a puromycin resistance gene, separated by a T2A cleavage sequence; and an EF1A promoter driving the expression of the overexpression transcript ORF.

The AREG overexpression vector coded for the ORF of transcript NM_001657.4, with the nucleotide sequence shown in SEQ ID NO:6. The ORF stuffer control was coded by the sequence shown in SEQ ID NO:7. All plasmid sequences were confirmed by Sanger sequencing.

### Example 3

The disclosed method reveals differences in the intercellular proteome between cancer types and is compatible with solid tumor cells of different origins. To establish the generalizability of the disclosed method the capabilities of the disclosed method were evaluated in 6 different models: (1) the triple-negative HCC70 breast cancer cell line, (2) the ER+ MCF7:WS8 cancer cell line, (3) the ER+ tamoxifen resistant TAMR cell line (which is derived from the MCF7:WS8 parental line), (4) the small-cell lung cancer DMS53 cancer cell line, (5) the small-cell lung cancer NCI-H1048 cell line, and (6) ex vivo organoid culture generated from a triple-negative breast cancer patient-derived xenograft tumor model (JAX PDX J000106527). Each of the cell lines were cultured in a 2% EHS matrix suspension as 3D organoid models, while the patient-derived xenograft organoids were cultured in an elastic EHS matrix in fully embedded 3D culture.

Each cell model in 3D culture was treated with 500 pM azidohomoalanine for 24 hours and then labeled with DBCO-PEG4-Desthiobiotin. The cells were lysed, and desthiobiotin-labeled proteins were immobilized on streptavidin magnetic beads. After washing to remove non-specifically bound proteins, the immobilized proteins were eluted in full-length form by mild elution with excess free biotin (3 mg/mL). The relative abundance of each protein in a 3072-plex panel was quantified using the proximity extension assay (PEA) according to the Olink® Explore protocol.

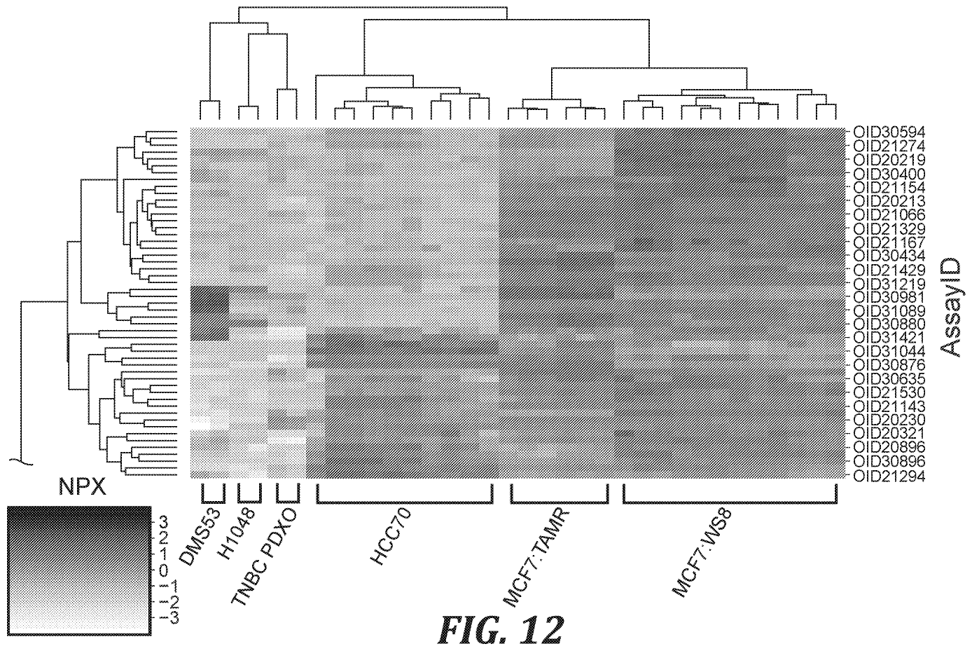

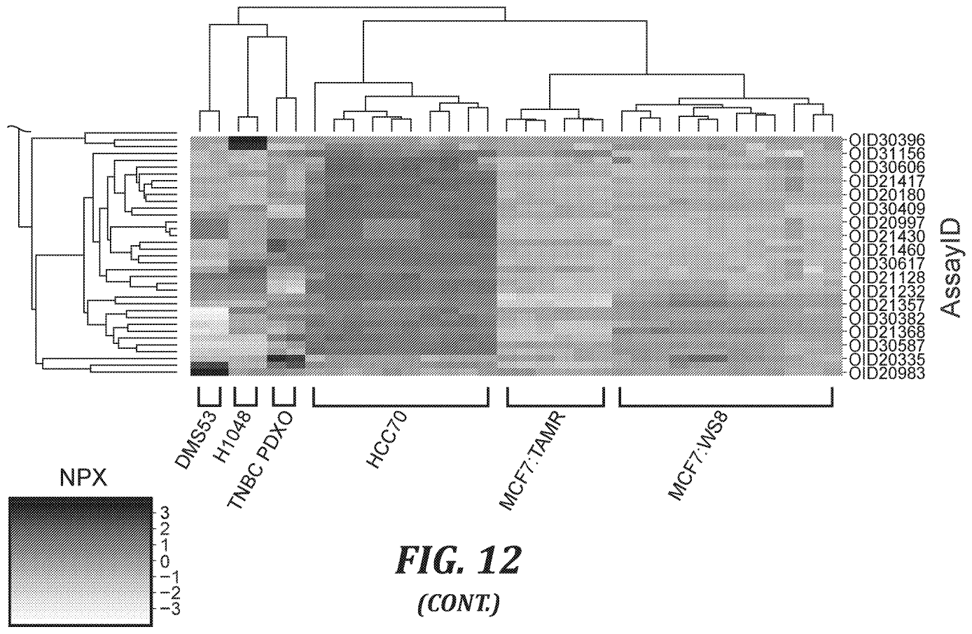

Microarray linear modeling analysis was performed to quantify and compare differential intercellular protein abundance across each cancer model. Comparing each cancer model, statistically significant differences in intercellular protein expression were identified. To evaluate the structure of these relationships on a more global scale, a hierarchical clustering analysis was performed (FIG. 12) on the 100 proteins that were significantly more abundant between models (as determined by an F-test from linear modeling analysis). This analysis revealed that breast cancer cell lines clustered together while all small-cell lung cancer models clustered together. At the next level of hierarchical clustering, all samples within each specific cancer model (e.g., HCC70 TNBC) clustered together, while distinct cancer models organized into separate clusters. Taken together, these results demonstrate that the method of the invention can identify and quantify hierarchical relationships among samples derived from distinct cancer models, suggesting that it can be used to discover tissue- or subtype-specific therapeutic targets.

### Example 3 Materials and Methods

Click chemistry and biotinylation reagents. L-Azidohomoalanine and DBCO-PEG4-Desthiobiotin were purchased from Click Chemistry Tools (cat. #1066-100, #1108-25, respectively). L-Azidohomoalanine was reconstituted in anhydrous DMSO at 500 mM. DBCO-PEG4-Desthiobiotin was reconstituted in anhydrous DMSO at 300 mM. All labeling reagents were stored at −20 degrees Celsius under desiccated conditions.

Cell lines. NCI-H1048 and DMS53 cells were originally purchased from the American Type Culture collection (ATCC) and were obtained as a gift from David MacPherson (Fred Hutchinson Cancer Center, Seattle, Washington). MCF7:WS8 cells were obtained as a gift from Donald McDonnell (Duke University, Durham, North Carolina) and their generation is described by Pink et al., supra. MCF7:TAMR cells were derived from the MCF7:WS8 cell line, and their generation is described by Cocce et al., supra. HCC70 cells were purchased from ATCC.

2D Monolayer culture of cell lines. Prior to generation of spheroids, all cell lines were maintained and propagated in 2D monolayer culture on tissue culture-treated flasks in a tissue-culture incubator at 37° C. and 5% CO₂. MCF7:WS8 cells were maintained in DMEM/F12 media supplemented with 8% fetal bovine serum, 1% penicillin-streptomycin, 1 mM sodium pyruvate, 2 mM GlutaMax, and MEM non-essential amino acid supplement (1:100 dilution). Media was changed every 2-3 days and cells were passaged upon reaching 70-80% confluence. NCI-H1048 cells and DMS53 cells were maintained in HITES media consisting of DMEM/F12, 1 mM sodium pyruvate, insulin-transferrin-selenium (10, 5.5, and 0.0067 g/ml) 30 nM hydrocortisone, 10 nM (3-estradiol, 1% pen/strep, 10% FBS.

Cells were passaged by dissociating with TrypLE expression at 37° C., followed by 5:1 dilution with complete cell culture media, centrifugation at 200×g for 3 minutes, and resuspension in complete cell culture media. Cells were propagated following dissociation at a split ratio in the range of 1:4 to 1:6.

Preparation of 2⁰% EHS matrix suspension media. 2% EHS matrix suspension media was obtained by combining ice-cold EHS matrix (Cultrex reduced growth factor basement membrane extract, R&D systems #3433-005-01) with ice-cold complete cell culture media (i.e., the culture media used for 2D cell propagation for the specific cell line of interest) at a ratio of 1:49 (for example, 1 mL of EHS matrix at the manufacturer's delivered concentration into 49 mL complete cell culture media). The mixture was immediately mixed well by inverting, and the EHS-supplemented media was then pre-warmed at 37° C. in a water bath to allow the EHS matrix to form crosslinks. A methionine-reduced variation of 2% EHS matrix media was prepared as above, except that DMEM/F12 media was replaced with methionine-free DMEM/F12.

Generation of cell line 3D spheroid cultures in 2% EHS matrix. The process for producing 3D spheroid cultures was the same for all cell lines, except that the complete culture media used for the suspension culture differed between the cell lines as indicated in the methods above for propagating in 2D monolayer culture.

Prior to generation of 3D spheroid cultures, all cells were passaged twice after revival from liquid nitrogen cryostorage to minimize effects associated with thawing. Cells were dissociated from 2D TC-treated culture flasks using TrypLE express by incubation for about 5 minutes at 37° C., or until cells fully detached from the surface. The cells were recovered from the flask and diluted 5:1 with complete cell culture media. Cells were pelleted by centrifugation at 200×g for 3 minutes and gently resuspended in phosphate buffered saline (PBS) without calcium or magnesium. Cells were counted with an automated cell counter hemocytometer (Countess). Next, the desired number of cells was pelleted by centrifugation at 200×g for 3 minutes and cells were resuspended in 2% EHS suspension media (prepared according to the recipe above) at a cell density of 150 thousand cells per mL of suspension media. The cell suspension was plated in 6-well ultra-low attachment plates (Corning #3471), and multicellular clusters were allowed to form. Spheroids were dissociated once after 4 days of initial establishment before being plated for experiments.

To dissociate spheroids, spheroids were pelleted by centrifugation at 200×g for 3 minutes and washed twice with PBS (without calcium, magnesium). Cells were then resuspended in Accumax cell dissociation solution (Innovative Cell Technologies #AM105) and incubated in a 37° C. water bath for 5 minutes. The spheroids were triturated with a 5 mL serological pipette ten times. The spheroids were then incubated at room temperature in the dissociation solution, and every 5 minutes the suspension was triturated with a 1 mL micropipette ten times. This process was repeated for up to 30 minutes until at least 80% of the cells were observed to be a single cell suspension when visualized under a phase contrast microscope.

Following dissociation, cells were pelleted at 200×g for 3 minutes and resuspended in PBS (without calcium, magnesium). Cells were counted on a hemocytometer using an automated cell counting system (Countess) and resuspended in 2% EHS matrix suspension media (recipe described above) at a cell density of 150 thousand cells per mL. The cell suspension was plated in 6-well ultra-low attachment plates (Corning #3471), and multicellular clusters were allowed to form.

Estrogen deprivation, azidohomoalanine, and drug treatment of MCF7: WS8 cells in 2% EHS matrix suspension culture. MCF7:WS8 cells were seeded, as described above, in estrogen-depleted 2% EHS matrix suspension culture (containing charcoal-stripped FBS and phenol red free) for 24 hours to create estrogen-starved spheroids. Spheroids were pelleted by centrifugation at 200×g for 3 minutes, then resuspended (at their original spheroid density) in estrogen-depleted 2% EHS matrix culture media containing 1 nM estradiol. Cells were cultured for an additional 24 hours, then pelleted by centrifugation and washed three times with PBS (containing calcium, magnesium) to remove excess methionine. The spheroids were then resuspended in methionine-reduced, estrogen-depleted 2% EHS matrix media containing azidohomoalanine (five hundred micromolar) and one of the above drug treatments. Spheroids were cultured for an additional 24 hours before proceeding to DBCO-PEG4-Desthiobiotin labeling.

DBCO-PEG4-Desthiobiotin and DBCO-sulfo-Biotin labeling in 2% EHS matrix suspension culture. After 24 hours of azidohomoalanine labeling, spheroids were washed three times with PBS (containing calcium and magnesium) to remove unincorporated azidohomoalanine by centrifugation at 200×g for 1 minute, aspiration of the supernatant, and resuspension in the PBS wash buffer. The spheroids were then resuspended in a PBS solution containing 1% bovine serum albumin and DBCO-PEG4-Desthiobiotin at a concentration of 300 pM. The cells were incubated at 37° C. in 6-well ultra-low-adhesion plates for 30 minutes in a tissue culture incubator maintained at 5% CO₂ to allow the SPAAC reaction to proceed. Then excess unreacted reagent was removed by washing the cells four times with PBS (containing calcium and magnesium) by repeated centrifugation at 4° C. (200×g, 1 minute) and resuspension. Samples were then pelleted by centrifugation at 200×g for 3 minutes, the supernatant was removed, and pellets were snap-frozen in liquid nitrogen by submerging the tube for 5 minutes. Snap-frozen pellets were stored at −80° C. until ready for use.

Preparation of DBCO-PEG4-Desthiobiotin labeled spheroid lysates. Prior to thawing spheroid samples, a lysis buffer consisting of 0.1% SDS, 1% Triton X-100, 150 mM NaCl, 20 mM Tris, 1 mM EDTA, pH 7.4 was freshly prepared and supplemented with protease inhibitors (Halt Protease inhibitor cocktail, 100× dilution, Thermo Fisher Scientific #87785) and phosphatase inhibitors (Halt Phosphatase inhibitor cocktail, 100× dilution, Thermo Scientific #78420). Spheroid pellets were thawed on ice in direct contact with ice-cold lysis buffer (one hundred microliters of lysis buffer per one million cells seeded for the spheroids). The mixture was periodically vortexed to facilitate thawing until the pellet was fully thawed. Once the pellets were fully thawed, samples were briefly vortexed to fully resuspend the cells, and samples were sonicated using a probe sonicator with six pulses, 0.5 seconds each. Samples were then incubated with gentle orbital agitation at 4° C. for 1 hour. After incubation, samples were again sonicated using a probe sonicator with six pulses, 0.5 seconds each. Next, samples were centrifuged in a pre-chilled microcentrifuge at 4° C. at 12,000×g for 10 minutes to remove insoluble material. The supernatant (representing the cleared spheroid lysate) was collected and used for the next step of the enrichment.

Patient-derived xenograft model establishment and passaging. The patient-derived xenograft model was originally purchased from Jackson Labs JAX PDX J000106527. For revival, cryopreserved PDX tumor fragments from Jackson Labs were encapsulated in a drop of Matrigel and orthotopically implanted into the mammary fat pads of immunocompromised mice. Mice were housed in sterile cages on a 12-hour light/dark cycle and tumors were measured by calipers at least weekly. Tumors were passaged by surgically removing the tumor, dissecting it into fragments, and re-embedding Matrigel-encapsulated fragments into the mammary fat pads of a new immunocompromised mouse host. Tumors were harvested for organoid generation once tumors reached a size of about 1.5 cm in diameter.

Organoid generation from patient-derived xenograft tumor models. Tumors were harvested when they were approximately 1.5 cm in diameter. The tumor was dissected from the mammary fat pad and mechanically dissected into fragments with a scalpel. A digestion solution was freshly prepared consisting of 2 mg/mL collagenase (Sigma C2139), 2 mg/mL trypsin (GIBCO 7250-018, 5% fetal bovine serum, five ug/mL insulin (Sigma-Aldrich I9278), and fifty ug/mL gentamicin (GIBCO 15750-060). Tumor fragments were incubated in digestion solution for 30 minutes to 1 hour at 37° C. while shaking at 150 RPM. Tumor fragments were collected by centrifugation at 453×g for 10 minutes. The remove genomic DNA, fragments were resuspended in 4 mL of DMEM/F12 containing 40 μL of a (2000 U/mL) DNA stock solution (Sigma #D4263) for 3 minutes. The suspension was centrifuged at 453×g for 4 seconds. To remove single cells and enrich for heavier multicellular fragments, the suspension was repeatedly centrifuged at 453×g for 4 seconds each, followed by resuspension in 10 mL DMEM/F12. This was repeated four times.

Organoids were collected by centrifugation at 453×g for 4 seconds, then resuspended in organoid media (Advanced DMEM/F12, 5% FBS, 10 mM HEPES, 1X GlutaMax, 1 μg/mL hydrocortisone, 50 μg/mL gentamicin, 10 ng/mL hEGF, 10 μM Y-27632) that was supplemented with 2% EHS matrix (1 mL Cultrex reduced growth factor basement membrane extract per 50 mL total media volume). Organoids were plated in ultra-low adhesion 6-well plates and cultured at 37° C., 5% CO₂ for 24 hours prior to embedding in elastic EHS matrix hydrogel.

Organoid 3d embedded culture. Following 24-hours of suspension culture (as described above), freshly prepared organoids were embedded in EHS matrix (Cultrex reduced growth factor basement membrane extract) elastic hydrogels. Organoids were pelleted by centrifugation at 200×g for 1 minute and the supernatant was removed. Organoids were washed once with PBS (without calcium, magnesium) by resuspension and 200×g for 1 minute, and a portion of the organoid suspension was aliquoted for cell counting. To count cells, an aliquot or organoid suspension was dissociated for 30 minutes in Accumax solution at room temperature. Cells were then counted using a hemocytometer and automated cell counter (Countess). Multicellular organoids (not subjected to dissociation solution) were then resuspended in ice-cold EHS matrix (Cultrex reduced growth factor basement membrane extract, protein concentration between 7-10 mg/mL) on ice at a cell density of 2.8 million cells per mL. The organoid suspension was pipetted up and down slowly until organoids were well-suspended. 30 μL EHS matrix domes containing organoids were then created by pipetting 30 μL EHS matrix directly into the center of a tissue-culture treated 24-well plate. The plate was inverted upside down to prevent organoid settling during gelation, and incubated at 37° C., 5% CO₂ for 1 hour to allow the matrix to solidify. After gelation, the matrix was submerged in organoid media (Advanced DMEM/F12, 5% FBS, 10 mM HEPES, 1X GlutaMax, 1 μg/mL hydrocortisone, 50 μg/mL gentamicin, 10 ng/mL hEGF, ten uM Y-27632), and the organoids were cultured at 37° C., 5% CO₂.

Azidohomoalanine and DBCO-PEG4-Desthiobiotin labeling of PDX organoids in 3D embedded culture. After 48 hours of culture, 3D embedded organoids were treated with azidohomoalanine. To remove excess methionine prior to labeling, the organoids were washed 4 times with methionine-free organoid media (Methionine-free DMEM/F12, 5% FBS, 10 mM HEPES, 1X GlutaMax, 1 μg/mL hydrocortisone, 50 μg/mL gentamicin, 10 ng/mL hEGF, 10 uM Y-27632), 10 minutes each at 37° C. with no agitation. Next, 1 mL of methionine-free media containing 500 pM azidohomoalanine was added to each well, and organoids were cultured at 37° C., 5% CO₂ for 24 hours.

After 24 hours of azidohomoalanine labeling, organoids were washed 4 times with methionine-free organoid media (Methionine-free DMEM/F12, 5% FBS, 10 mM HEPES, 1X GlutaMax, 1 μg/mL hydrocortisone, 50 μg/mL gentamicin, 10 ng/mL hEGF, 10 μM Y-27632), 30 minutes each at 37° C. to remove excess azidohomoalanine. During the washes, a DBCO labeling solution was freshly prepared consisting of methionine-free organoid media (Methionine-free DMEM/F12, 5% FBS, 10 mM HEPES, 1X GlutaMax, 1 μg/mL hydrocortisone, 50 μg/mL gentamicin, 10 ng/mL hEGF, 10 μM Y-27632), and 300 μM DBCO-PEG4-Desthiobiotin, and pre-warmed to 37° C. EHS matrix domes were submerged in 1 mL of the DBCO labeling solution per well and incubated for 30 minutes at 37° C. while agitating at 80 RPM on an orbital shaker. To remove excess DBCO labeling reagent, samples were washed once with ice-cold methionine-free organoid media, followed by three washes with ice-cold PBS (with calcium, magnesium), each for 10 minutes at 4° C. with gentle orbital agitation. After washing, organoid containing EHS matrix domes were immediately subjected to lysis.

Organoid containing EHS matrix gel cultures were lysed with a lysis buffer consisting of 0.1% SDS, 1% Triton X-100, 150 mM NaCl, 20 mM Tris, 1 mM EDTA, pH 7.4 was freshly prepared and supplemented with protease inhibitors (Halt Protease inhibitor cocktail, 100× dilution, Thermo Fisher Scientific #87785) and phosphatase inhibitors (Halt Phosphatase inhibitor cocktail, 100× dilution, Thermo Scientific #78420). For every million cells (representing twelve wells of thirty microliter EHS matrix domes), 500 μL of lysis buffer were used. Each dome was collected into the lysis buffer in a serial manner by pipetting the lysis buffer solution into the well and vigorously pipetting up and down. Samples were sonicated using a probe sonicator with six pulses, 0.5 seconds each. Samples were then incubated with gentle orbital agitation at 4° C. for 1 hour. After incubation, samples were again sonicated using a probe sonicator with six pulses, 0.5 seconds each. Next, samples were centrifuged in a pre-chilled microcentrifuge at 4° C. at 12,000×g for 10 minutes to remove insoluble material. The supernatant (representing the cleared spheroid lysate) was collected and used for the next step of the enrichment.

Streptavidin Magnetic Bead Enrichment. One micron-sized magnetic polystyrene beads covalently functionalized with streptavidin (binding potential: 2,500 pmoles free biotin/mg beads), bearing a hydrophilic, carboxylated surface (Streptavidin MyOne Cl, Thermo Fisher cat #65001), and a magnetic separation rack (DynaMag 2, Thermo Fisher Scientific cat #12321D) were used to enrich proteins that had been covalently labeled with either a biotin or desthiobiotin enrichment handle. For every million starting cells contained in the lysate, two hundred microliters of the 10 mg/mL magnetic bead suspension were used. Prior to use, the beads were washed three times with a tris-buffered saline wash buffer (TBST) consisting of 150 mM NaCl, 20 mM Tris, 0.2% Tween-20, pH 7.6 by separating the beads on the magnetic stand, removing the supernatant, and resuspending the beads in 500 μL of TBST. Next a magnetic bead pellet was prepared by separating the beads on a magnetic stand and removing the supernatant. The cleared spheroid lysate was added directly to the magnetic bead pellet, and the beads were resuspended in the lysate by dragging the tube along a microtube rack repeatedly and relying on the vibrations generated by the uneven microtube rack surface (This method of dragging the tube along the microtube rack was found to be superior to vortexing). The bead/lysate mixture was then incubated for 3 hours at room temperature with orbital agitation to prevent the beads from settling. Next, the magnetic beads were separated on the magnetic stand, and the supernatant was collected and kept as the “unbound fraction.” The beads were then washed three times with TBST by repeatedly resuspending the pellet (by running the tube along a microtube rack) and then separating the magnetic beads. No incubation time was applied during the washes.

To elute bound proteins from the beads, an elution buffer containing 0.1% SDS, 1% Triton X-100, 150 mM NaCl, 20 mM Tris, 1 mM EDTA, 3 mg/mL biotin was prepared. Biotin free acid is only soluble in water in its deprotonated form, so a two times concentrated form of the buffer was initially prepared and titrated with sodium hydroxide until all biotin fully dissolved and the pH reached 7.4-7.6. The buffer was then diluted with MilliQ water to reach the composition. 50 μL of elution buffer were used per 100 μL of starting lysate material. The pelleted beads, containing the immobilized proteins, were resuspended in the elution buffer by dragging the tube along a microtube holder. The samples were first incubated for 2 hours at 37° C. while shaking at 200 RPM, followed by a 16-hour incubation at 4° Celsius, with gentle orbital agitation. Next, the magnetic beads were separated on a magnetic stand, and the supernatant was collected as the final elution fraction. The eluted fraction was snap-frozen by submerging the tube in liquid nitrogen for 5 minutes, and the sample was stored at −80° C. until ready for analysis by the proximity extension assay (PEA).

Intercellular protein discovery by the proximity extension assay and NPX normalized protein abundance calculation. Proteins were measured using the Olink® Explore 3072 panel (Olink Proteomics AB, Uppsala, Sweden) according to the manufacturer's instructions. For each experimental replicate, 80 μL of elution solution (representing labeled proteins from eight hundred thousand cells), was used directly for the Olink Explore protocol. The Olink Explore 3072 panel uses a 3072-plex panel of oligonucleotide-conjugated antibody probe pairs. Each antibody probe pair is designed to bind to the target, resulting in hybridization of the complementary oligonucleotides provided by each member of the paper. DNA polymerase is then used to extend only oligonucleotides that have hybridized, producing a unique DNA barcode each time a protein is bound to both members of an antibody pair whose conjugated oligonucleotides were designed to hybridize. Library preparation was then performed to add sample identification indices and the necessary nucleotides for illumine sequencing. Libraries were subjected to a bead-based purification and quality was assessed using an Agilent 2100 bioanalyzer (Agilent Technologies, Palo Alto, CA). Samples were then subjected to next-generation sequencing using the Illumina NovaSeq 6000. The data was then quality controlled and converted into Normalized Protein eXpression (NPX) values, which represent Olink's unit of relative abundance. For quality control, three internal controls were spiked into each sample and used to evaluate the performance of 3 steps in the protocol: an incubation control with a non-human antigen and its matching antibody probes, an extension control consisting of an IgG antibody conjugated to a pair of hybridizing oligonucleotides, and an amplification control consisting of a complete double-stranded DNA amplicon. The Olink Explore protocol was also performed on a set of external controls: two different pooled human plasma samples, three negative controls, and three plate controls. Each plate control is a plasma sample that is used to normalize across plates.

The NPX value was calculated by first computing ExtNPX(assay I, sample J), according to ExtNPX(assay i, sample j)=log 2(counts(assay i, sample j)/(counts(Extension control, sample j)); which normalized the counts of each sample to the counts of a known standard. Next the NPX value was computed as NPX(assay i, sample j)=ExtNPX(assay i, sample j)−median (ExtNPX(PC, assay i)); which normalized across plates. The limit of detection for each sample was defined to be equal to an NPX value that is three standard deviations above the median of the negative control samples. All proteins below the LOD were excluded from the list of discovered proteins. All assay validation data (detection limits, intra- and inter-assay precision data, predefined values, etc.) are available on manufacturer's website.

Differential expression analysis of PEA quantification data. Differential expression analysis to identify statistical differences in protein expression between cell lines was performed using the limma (v. 3.50.3) and edgeR (v.3.36.0) packages in R (v. 4.1.1). Specifically, the matrix of NPX values was fitted to a linear model of the form y Cell Line using limma. The linear fit coefficients were subjected to empirical Bayes smoothing using the eBayes function in edgeR. Contrasts in the linear modeling coefficients were then fitted using limma and subjected to empirical Bayes smoothing to obtain the log 2-fold change, t-statistic, and unadjusted p-value. The p-values were then corrected for multiple comparisons using the Benjamini and Hochberg false-discovery rate correction procedure to obtain the final FDR.

Hierarchical clustering analysis of top differentially expressed genes between cell lines. The matrix of NPX values was fitted to a linear model of the form y~Cell Line using limma. From the linear modeling analysis, fit coefficients associated with an effect due to cell line, and their associated F-statistic values, were obtained. The reader should note that this analysis provides the statistical evidence for the fact that an effect on expression exists due to changing cell type but does not describe the statistical evidence for the contrast between two particular cell types, and instead provides a means of a global-level ranking of genes by those that are most likely to vary among at least some cell lines. Proteins were ranked by F-statistic to obtain a list of proteins ranked by strongest evidence for an effect due to type. The top one hundred proteins, ranked by F-statistic, were then subjected to hierarchical clustering analysis using the cluster map function in the Seaborn (v. 0.12.2) package with Python (v. 3.8.8). NPX normalized abundances were Z-scored on a per-gene basis and the Z-scored NPX values were hierarchically clustered based on the Euclidean distance metric with the Nearest Point Algorithm.

### Example 4

Mutant t-RNA synthetase can be used to enable pericellular click labeling only in cells that have been genetically engineered to express the mutant t-RNA synthetase. The disclosed method enables proteins that are newly synthesized within a culture system to be distinguished from exogenous protein supplements (such as EHS matrix). However, in a heterogenous culture system or living tissue, where multiple different cell types are actively synthesizing new proteins, the currently disclosed method alone does not enable differentiation between proteins that are newly synthesized by different subpopulations. This is because azidohomoalanine, an amino acid analog, is recognized by the wild-type t-RNA synthetase incorporated by all cells with active protein synthesis. However, t-RNA synthetase can be engineered to recognize only specific amino acid analogs, which are unrecognized by wild-type t-RNA synthetases. Consider, for example, methionyl-tRNA synthetase (MetRS), which covalently links methionine to its cognate tRNA in protein biosynthesis. Mutant versions of MetRS incorporate azidonorleucine, a noncanonical amino acid, rather than methionine into protein biosynthesis (see, e.g., Link et al., Proc. Natl. Acad. Sci. USA 103:27, 2006). Because azidonorleucine is not a substrate of endogenous aminoacyl-tRNA synthase, azidonorleucine-labeling is only localized to cells that express mutant MetRS. Thus, mutant t-RNA synthetases can be expressed in specific cells, e.g., under the control of Cre recombinase or cell-specific promoters, to enable cell-specific click labeling. However, prior attempts have not combined this capability with the ability to specifically target the intercellular proteome and recover full-length proteins.

Therefore, the aim was to demonstrate the ability of the disclosed method to specifically label the pericellular proteome in a mutant-specific manner using methods that would be compatible with full-length protein recovery. Using the MCF7:WS8 ER+ breast cancer cell line, a cell line that constitutively overexpressed the mutant methionine t-RNA synthetase, L274GMmMetRS, was generated by lentiviral transduction and puromycin selection. The L274GMmMetRS t-RNA synthetase is derived from the Mus musculus methionine t-RNA synthetase by introducing a single residue mutation at the L274 leucine reside, converting it to a glycine residue. This mutation enables it to selectively incorporate the azide-bearing amino acid analog, azidonorleucine in place of methionine residues.

Three-dimensional cultures of MCF7:WS8:metRS-wildtype and MCF7:WS8:metRS-mutant spheroids were generated in 2% EHS matrix suspension culture. After spheroids were established, the cells were cultured in the presence of 500 μM azidonorleucine in methionine-containing media for 24 hours. Spheroids were then labeled for 30 minutes with 300 μM DBCO-PEG4-Desthiobiotin and fixed using paraformaldehyde/glutaraldehyde-based fixation. To visualize the proteins that had been modified with the DBCO-PEG4-Desthiobiotin, the cells were permeabilized and labeled with streptavidin-alexafluor-488. Cells were then counterstained with DAPI to visualize the nucleus.

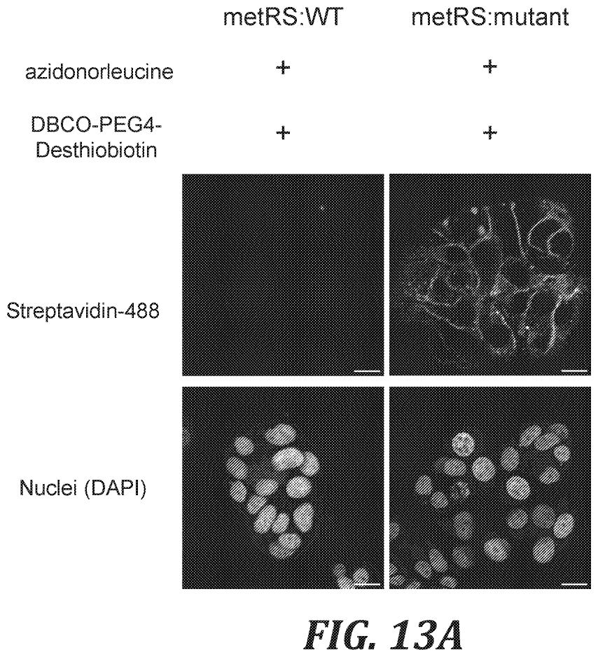

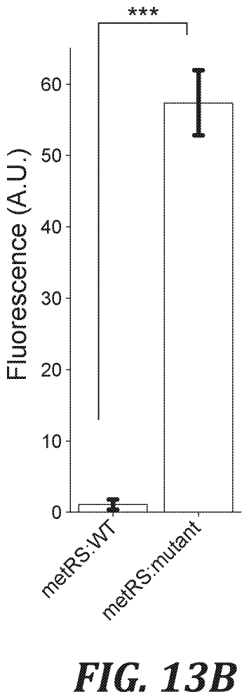

Streptavidin-alexafluor-488 labeling in both mutant and wildtype cells was assessed by confocal fluorescence microscopy (FIG. 13). The cell lines engineered with the L274GMmMetRS exhibited bright fluorescent staining that localized to intercellular zones. In contrast, staining in the untransduced metRS wildtype cells was virtually undistinguishable from background fluorescence. To more quantitatively assess the specificity of labeling, the fluorescence intensity in at least 10 different fields of view was quantified across 3 replicates of the experiment. In each field of view, the DAPI nuclear stain was captured. To normalize the fluorescence intensity against the number of cells, the total area of the threshold DAPI nuclear signal was computed and divided by the streptavidin-alexafluor-488 signal against the total nuclear area for each sample.

This analysis revealed that the cells engineered to express the L274GMmMetRS mutant exhibited over 50-fold higher normalized streptavidin-alexa-fluor-488 fluorescence intensity compared to the wildtype cells. These results suggest that in a heterogeneous culture system, proteins produced from cells bearing a L274GMmMetRS mutation could be distinguished from cells expressing the wildtype t-RNA synthetase at a high signal to background. Moreover, the qualitative localization analysis confirmed that this labeling is restricted to the pericellular space, and the disclosed method would also be compatible with streptavidin magnetic bead enrichment, full-length protein elution, and other downstream immunoaffinity-based detection methods.

### Example 4 Materials and Methods

Click chemistry and biotinylation reagents. L-Azidonorleucine hydrochloride was purchased from Tocris (cat. #6585) and resuspended in anhydrous DMSO at a concentration of 500 mM. DBCO-PEG4-Desthiobiotin was purchased from Click Chemistry Tools (cat. #1108-25). DBCO-PEG4-Desthiobiotin was reconstituted in anhydrous DMSO at 300 mM. All labeling reagents were stored at −20 degrees Celsius under desiccated conditions.

MCF7: WS8 2D monolayer culture. Prior to generation of spheroids, MCF7:WS8 cells and genetically modified versions thereof were maintained and propagated in 2D monolayer culture on tissue culture-treated flasks in a tissue-culture incubator at 37° C. and 5% CO₂. Cells were maintained in DMEM/F12 media supplemented with 8% fetal bovine serum, 1% penicillin-streptomycin, 1 mM sodium pyruvate, 2 mM GlutaMax, and MEM non-essential amino acid supplement (1:100 dilution). Media was changed every 2-3 days and cells were passaged upon reaching 70-80% confluence. Cells were passaged by dissociating with TrypLE expression at 37° C., followed by 5:1 dilution with complete cell culture media, centrifugation at 200×g for 3 minutes, and resuspension in complete cell culture media. Cells were propagated following dissociation at a split ratio in the range of 1:4 to 1:6.

Preparation of 2% EHS matrix suspension media. 2% EHS matrix suspension media was obtained by combining ice-cold EHS matrix (Cultrex reduced growth factor basement membrane extract, R&D systems #3433-005-01) with ice-cold complete cell culture media (DMEM-fl2 media containing 8% fetal bovine serum, 1% penicillin-streptomycin, 2 mM GlutaMax, 1:100 MEM non-essential amino acids, and 1 mM sodium pyruvate) at a ratio of 1:49 (for example, 1 mL of EHS matrix at the manufacturer's delivered concentration into 49 mL complete cell culture media). The mixture was immediately mixed well by inverting, and the EHS-supplemented media was then pre-warmed at 37° C. in a water bath to allow the EHS matrix to form crosslinks. For azidonorleucine labeling, methionine-containing media was used since methionine does not compete for the mutant t-RNA synthetase with azidonorleucine.

Generation of MCF7: WS8 3D spheroid cultures in 2% EHS matrix. Prior to generation of 3D spheroid cultures, MCF7 cells were passaged twice after revival from liquid nitrogen cryostorage to minimize effects associated with thawing. MCF7 cells were dissociated from 2D TC-treated culture flasks using TrypLE express by incubation for about 5 minutes at 37° C., or until cells fully detached from the surface. The cells were recovered from the flask and diluted 5:1 with complete cell culture media. Cells were pelleted by centrifugation at 200×g for 3 minutes and gently resuspended in phosphate buffered saline (PBS) without calcium or magnesium. Cells were counted with an automated cell counter hemocytometer (Countess). Next, the desired number of cells was pelleted by centrifugation at 200×g for 3 minutes and cells were resuspended in 2% EHS suspension media (prepared according to the recipe above) at a cell density of 150 thousand cells per mL of suspension media. The cell suspension was plated in 6-well ultra-low attachment plates (Corning #3471), and multicellular clusters were allowed to form. Spheroids were dissociated once after 4 days of initial establishment before being plated for experiments.

To dissociate spheroids, spheroids were pelleted by centrifugation at 200×g for 3 minutes and washed twice with PBS (without calcium, magnesium). Cells were then resuspended in Accumax cell dissociation solution (Innovative Cell Technologies #AM105) and incubated in a 37° C. water bath for 5 minutes. The spheroids were triturated with a 5 mL serological pipette ten times. The spheroids were then incubated at room temperature in the dissociation solution, and every 5 minutes the suspension was triturated with a 1 mL micropipette ten times. This process was repeated for up to 30 minutes until at least 80% of the cells were observed to be a single cell suspension when visualized under a phase contrast microscope.

Following dissociation, cells were pelleted at 200×g for 3 minutes and resuspended in PBS (without calcium, magnesium). Cells were counted on a hemocytometer using an automated cell counting system (Countess) and resuspended in 2% EHS matrix suspension media (recipe described above) at a cell density of 150 thousand cells per mL. The cell suspension was plated in 6-well ultra-low attachment plates (Corning #3471), and multicellular clusters were allowed to form.

Azidonorleucine labeling ofMCF7: WS8 cells in 2% EHS matrix suspension culture. MCF7 cells were seeded, as described above, in 2% EHS matrix suspension culture for 48 hours. Spheroids were pelleted by centrifugation at 200×g for 3 minutes, then resuspended (at their original spheroid density) in 2% EHS matrix culture media containing azidonorleucine (500 μM). Methionine-reduced media was not used. Spheroids were cultured for an additional 24 hours before proceeding to DBCO-PEG4-Desthiobiotin labeling.

DBCO-PEG4-Desthiobiotin labeling in 2% EHS matrix suspension culture. After 24 hours of azidohomoalanine labeling, spheroids were washed three times with PBS (containing calcium and magnesium) to remove unincorporated azidohomoalanine by centrifugation at 200×g for 1 minute, aspiration of the supernatant, and resuspension in the PBS wash buffer. The spheroids were then resuspended in a PBS solution containing 1% bovine serum albumin and DBCO-PEG4-Desthiobiotin at a concentration of 300 μM. The cells were incubated at 37° C. in 6-well ultra-low-adhesion plates for 30 minutes in a tissue culture incubator maintained at 5% CO₂ to allow the SPAAC reaction to proceed. Excess unreacted reagent was then removed by washing the cells four times with PBS (containing calcium and magnesium) by repeated centrifugation at 4° C. (200×g, 1 minute) and resuspension.

Spheroid fixation and streptavidinfluorescence detection ofDBCO-PEG4-Desthiobiotin labeled samples. Immediately following labeling with DBCO-PEG4-Desthiobiotin probes, the spheroids were pelleted by centrifugation (200×g, 1 minute) and resuspended the spheroids in a fixation solution consisting of PBS, 4% paraformaldehyde, and 0.1% glutaraldehyde. The spheroids were incubated in fixation solution for 10 minutes at room temperature with gentle agitation on an orbital shaker. After 10 minutes of incubation, the spheroids were allowed to settle by gravity, removed excess fixation solution, and washed the spheroids once with a quenching buffer consisting of PBS and 200 mM glycine. After removing excess quenching solution, the spheroids were resuspended a second time in the quenching buffer and incubated the spheroids for 20 minutes at room temperature to quench unreacted aldehydes.

To prepare the spheroids for secondary detection desthiobiotin, the cell membrane was permeabilized for 1 hour at room temperature with a solution of PBS containing 0.1% Tween-20. Next, non-specific binding was blocked using a solution of PBS containing 0.1% Tween-20 and 10% FBS for 1 hour at room temperature. Proteins were detected that had undergone a labeling reaction using Alexafluor 568-conjugated streptavidin (streptaviding-568). Samples were incubated with a solution of PBS containing streptavidin-488 at a concentration of 0.2 mg/mL (1,000× dilution of a 2 mg/mL stock) and 0.1% (v/v) tween-20 for 16-24 hours at 4° C. Samples were then washed for 10 minutes with PBS containing 0.1% tween-20, 4 times. Spheroids were counterstained to label nuclei with DAPI and in PBS with 0.1% tween-20 for 1 hour at room temperature. Samples were then washed for 10 minutes with PBS containing 0.1% tween-20, 4 times. Finally, samples were allowed to settle by gravity, the wash buffer was aspirated, and spheroids were suspended in PBS (containing calcium, magnesium) and stored at 4° C. for up to 4 days prior to imaging.

Confocal fluorescence microscopy analysis of DBCO-PEG4-Desthiotiobin Streptavidin-488 labeling in t-RNA synthetase mutant and wild type MCF7 cells. Confocal mages were acquired using an Andor CSU-W confocal spinning disk on a Leica DMi8 inverted microscope using a 40X water immersion objective. In three separate experimental replicates for each cell line, fifteen fields of view were collected from each replicate to capture the Streptavidin-568, DAPI, and GFP, and a 50-micron z-stack was collected in each field of view, centered on the middle of organoids in the field of view. Care was taken to ensure that fluorescence intensity values were within the dynamic range of the detector and no pixels were saturated.

Analysis was performed using custom analysis script written in Python with the scikit-image package. The cumulative streptavidin-568 fluorescence intensity was computed across all fields of view for each experimental replicate. To normalize by the number cells, the maximum-z-projection of the DAPI signal was thresholded using an Otsu threshold, and the average threshold area was measured. The integrated streptavidin-568 fluorescence intensity was normalized to the DAPI threshold area for each replicate.

Plasmidpreparationfor lentiviral vectors. All plasmids were purchased in the form of bacterial glycerol stocks. Glycerol stocks were streaked onto agar plates containing 100 μg/mL ampicillin and incubated for 16 hours. An isolated colony was selected from the plate and cultured in a 5 mL Luria-Bertani (LB) starter culture containing 100 μg/mL ampicillin. After 8 hours, a portion of the starter culture was diluted 1:500 into 100 mL LB media 100 μg/mL ampicillin for inoculation. Culture was performed while shaking at 250 RPM at 37° C. for 16 hours. After 16 hours, bacterial pellets were harvested by centrifugation at 4,000×g for 10 minutes at 4° C., and plasmids were purified using a Qiagen Highspeed Plasmid Midi Kit (Qiagen #12643) according to the manufacturer's protocol. Plasmids were resuspended in ultra-pure nuclease-free water and their concentration and purity was determined spectrophotometrically using a NanoDrop.

Lentiviral particle generation. Lentiviral particles were generated by transfection of HEK-293FT cells. HEK-293FT cells were cultured in high glucose DMEM with 10% FBS, GlutaMax 1% penicillin-streptomycin, and 500 μg/mL Genetecin. HEK-293FT cells were passaged twice prior to transfection and allowed to reach 80% confluence. Upon reaching 80% confluence, cells were co-transfected with lentiviral vectors and packaging plasmids (3 μg MD2.G, 8 μg PsPax, 12 μg lentiviral vector containing the plasmid of interest). Lipoplexes were formed containing the three plasmids by combining with Lipofectamine 3000 (Thermo Fisher Scientific #L3000015) according to the manufacturer's instructions. The media on HEK-293FT cells was replaced with serum-free Opti-MEM media (Gibco #31985062), and the lipoplex solution was added dropwise. Cells were incubated in contact with the lipoplex solution for 16 hours at 37° Celsius, 5% CO₂.

After 16 hours, the transfection media was removed and replaced with complete cell culture media (minus the Genetecin). 24 hours after replacing the media, the supernatant (containing lentiviral particles) was collected. The supernatant was cleared of cellular debris by centrifugation at 2,000×g for 5 minutes, and the supernatant was collected and filtered through a 0.45-micron PVDF filter membrane. The lentiviral particles were concentrated 100-fold using the Lenti-X concentrator system according to the manufacturer's protocol (Takara Bio #631232). Concentrated lentiviral particles were then flash frozen by submerging the tube in liquid nitrogen and stored at −80 degrees Celsius until ready for use.

Lentiviral transduction. Prior to lentiviral transduction, a functional titer was performed (using the transduction protocol below) by transducing MCF7 cells with serial dilutions of lentiviral particles. Prior to transduction, MCF7 cells were plated on tissue-culture treated flasks at a cell density of 25,000 cells/cm². 24 hours after plating, the media was replaced with transduction media consisting of serum-free DMEM/F-12 with GlutaMax (1:100) and sodium pyruvate (1:100) supplemented with eight ug/mL protamine sulfate (1:500 dilution of 4 mg/mL stock). An appropriate volume of lentiviral particles representing a multiplicity of infection (MOI) of one was diluted into one hundred pL of PBS. The lentiviral particle dilution was then added dropwise to the media in the MCF7:WS8 cell culture, and the flask was mixed by gently swirling by hand. Cells were then incubated in contact with lentiviral particles for 24 hours at 37° C., 5% Co₂.

Twenty-four hours after adding lentiviral particles, the media was exchanged with complete cell culture media. Cells were cultured for an additional 48 hours before adding selection antibiotic. After 48 hours of culture in complete culture media, the media was exchanged with selection media, consisting of complete cell culture media containing 2 μg/mL puromycin. MCF7 cells were maintained in selection media and passaged upon reaching 80% confluence for 2 weeks prior to use in experiments. An untransduced control was subjected to the exact same culture conditions, and it was visually confirmed by phase contrast microscopy that all cells in the untransduced control had died in selection media.

Mutant L274GMmMetRS overexpression plasmid. The mutant L274GMmMetRS overexpression construct is based on a previously published ORF sequence. See Mahdavi et al., J. Am. Chem. Soc. 138:4278-4281, 2016. The plasmid was built on a pLV lentiviral plasmid backbone with a dual promoter cassette consisting of a pCMV-GFP-T2A-Puro expression cassette and an EF1A-metRS expression cassette. The L274GMmMetRS was coded by nucleotide sequence shown in SEQ ID NO:8.

### Example 5

Synthesis of DBCO-sulfo-Desthiobiotin. The structure of the click probe, DBCO-sulfo-Desthiobiotin, is as follows:

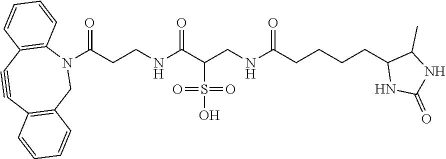 [SMILES: CC1NC(=O)NC1CCCCC(=O)NCC(C(=O)NCCC(=O)N1Cc2ccccc2C#Cc2ccccc21)S(=O)(=O)O]

DBCO-sulfo-Desthiobiotin is synthesized when N-Hydroxysuccinimide esters and primary amines react to form stable amide linkages in polar aprotic solvents. The reaction is catalyzed by the presence of a sterically hindered basic catalyst.

### Example 5 Materials and Methods

DBCO-sulfo-Desthiobiotin synthesis. All starting reagents are obtained commercially, including DBCO-sulfo-amine (Click Chemistry Tools cat. #1227-25), NHS-Biotin (Thermo Scientific #20217), and N,N-Diisopropylethylamine (Sigma-Aldrich #D125806).

DBCO-sulfo-PEG4-amine (0.02 mmol) is added to a round bottom, flame-dried flask and dissolved in 1 mL anhydrous DMF (N,N-Dimethylformamide) at room temperature. NHS-Desthiobiotin (0.02 mmol) is then added to the flask, followed by N,N-diisopropylethylamine (0.03 mmol). The reaction mixture is stirred continuously at room temperature for 16 hours. The reaction mixture is then diluted with brine (8:1) and extracted three times with ethyl acetate. The combined organic layers are then dried over anhydrous magnesium sulfate, filtered through a glass frit, and concentrated by rotary evaporation. The concentrated product is then purified by flash chromatography through a silica gel stationary phase using a mobile phase consisting of 0-10% methanol in methylene chloride. The fractions containing the desired product are identified by thin layer chromatography. The pooled fractions containing the desired product are concentrated by rotary evaporation to yield a clear to slightly yellow colored oil (expected yield~50%). The identity of the final product is confirmed by 1H NMR.

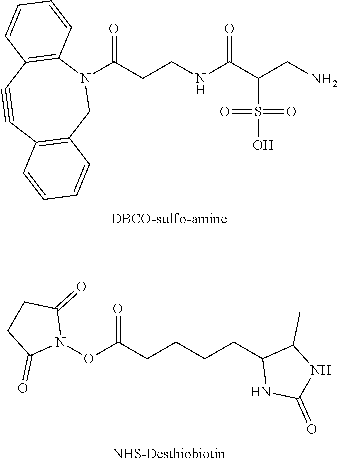 [SMILES: NCC(C(=O)NCCC(=O)N1Cc2ccccc2C#Cc2ccccc21)S(=O)(=O)O ; CC1NC(=O)NC1CCCCC(=O)ON1C(=O)CCC1=O]

### Example 6

Synthesis of DBCO-sulfo-PEG4-Desthiobiotin. The structure of the click probe, DBCO-sulfo-PEG4-Desthiobiotin, is as follows:

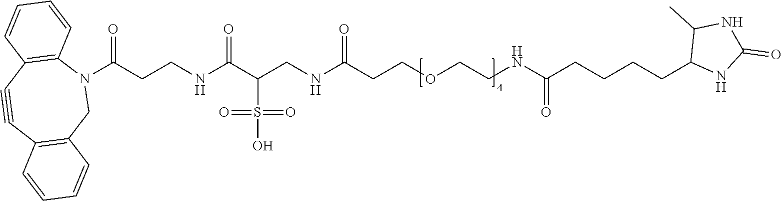

### Example 6 Materials and Methods

DBCO-sulfo-PEG4-Desthiobiotin synthesis. All starting reagents are obtained commercially, including DBCO-sulfo-PEG4-amine (Click Chemistry Tools cat. #1228-25), NHS-Biotin (Thermo Scientific #20217), and N,N-Diisopropylethylamine (Sigma-Aldrich #D125806).

DBCO-sulfo-PEG4-amine (0.02 mmol) is added to a round bottom, flame-dried flask, and dissolved in 1 mL anhydrous DMF (N,N,-Dimethylformamide) at room temperature. NHS-Desthiobiotin (0.02 mmol) is then added to the flask, followed by diisopropylethylamine (0.03 mmol). The reaction mixture is stirred continuously at room temperature for 16 hours. The reaction mixture is then diluted with brine (8:1) and extracted three times with ethyl acetate. The combined organic layers are then dried over anhydrous magnesium sulfate, filtered through a glass frit, and concentrated by rotary evaporation. The concentrated product is then purified by flash chromatography through a silica gel stationary phase using a mobile phase consisting of 0-10% methanol in methylene chloride. The fractions containing the desired product are identified by thin layer chromatography. The pooled fractions containing the desired product are concentrated by rotary evaporation to yield a clear to slightly yellow colored oil (expected yield~50%). The identity of the final product is confirmed by 1H NMR.

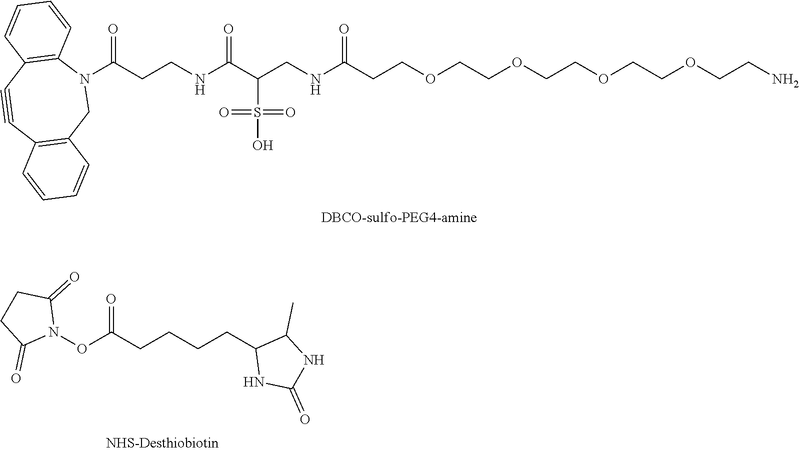 [SMILES: NCCOCCOCCOCCOCCC(=O)NCC(C(=O)NCCC(=O)N1Cc2ccccc2C#Cc2ccccc21)S(=O)(=O)O ; CC1NC(=O)NC1CCCCC(=O)ON1C(=O)CCC1=O]

While illustrative embodiments have been illustrated and described, it will be appreciated that various changes can be made therein without departing from the spirit and scope of the invention. All publications, patents, and patent applications cited herein are hereby incorporated by reference in their entireties for all purposes.

1. A method for isolating proteins that are pericellularly localized in a multicellular system, the method comprising:
(a) culturing a plurality of cells in a cell culture system, wherein the plurality of cells are cultured in the presence of an amino acid analog functionalized with a first chemical group that is reactive in a bioorthogonal chemical reaction, and wherein the amino acid analog is incorporated into newly synthesized proteins;
(b) contacting the cell culture system with a cell-membrane-impermeable probe having the formula A-L-D,
wherein
A is a molecule comprising a second chemical group that is reactive in a bioorthogonal chemical reaction, wherein the first chemical group is reactive with second chemical group,
L is a hydrophilic linker, and
D is a molecule comprising a biotin analog moiety capable of specifically binding to streptavidin with a dissociation constant greater than about 1 pM and an off-rate kinetic constant greater than about 1×10⁻³ l/s, and
wherein newly synthesized proteins that are pericellularly localized are labeled with the probe by a bioorthogonal chemical reaction between the first and second chemical groups;
(c) lysing the cultured cells to generate a lysate containing the labeled proteins and other cellular and extracellular components;
(d) contacting the lysate with a capture reagent comprising a biotin-binding protein immobilized on a solid support, wherein the biotin-binding protein binds to the biotin analog moiety on the labeled proteins to yield a [labeled protein]:probe complex; and
(e) separating the solid support from the lysate to separate the labeled proteins from the other cellular and extracellular components, thereby isolating the proteins that are pericellularly localized in the multicellular system.

2. The method of claim 1, wherein the biotin analog moiety is selected from the group consisting of a desthiobiotin moiety, an N3′-ethyl biotin moiety, and a 2-iminobiotin moiety.

3. The method of claim 2, wherein the biotin analog moiety has the formula D1, D2, or D3 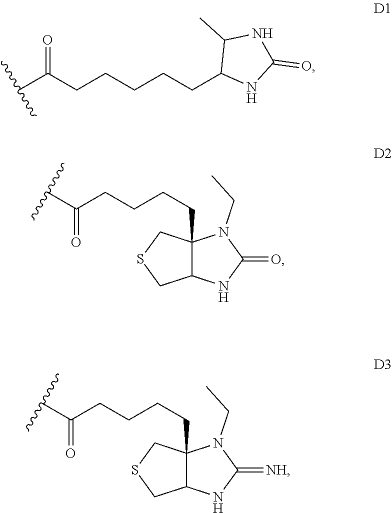 [SMILES: *C(=O)CCCCCC1NC(=O)NC1C ; *C(=O)CCCC[C@]12CSCC1NC(=O)N2CC ; *C(=O)CCCC[C@]12CSCC1NC(=N)N2CC]
wherein [symbol] designates a point of attachment.

4. The method of claim 1, further comprising eluting the labeled proteins from the immobilized biotin-binding protein, wherein the labeled proteins remain intact as full-length proteins following elution.

5. The method of claim 4, wherein eluting the labeled proteins from the immobilized biotin-binding protein comprises competitive elution with free biotin.

6. (canceled)

7. (canceled)

8. (canceled)

9. (canceled)

10. (canceled)

11. (canceled)

12. The method of claim 1, wherein the first chemical group that is reactive in a bioorthogonal reaction is an azide.

13. The method of claim 12, wherein the second chemical group that is reactive in a bioorthogonal chemical reaction is a strained alkyne.

14. (canceled)

15. (canceled)

16. The method of claim 13, wherein A comprises dibenzocyclooctyne (DIBO/DBCO).

17. The method of claim 1, wherein the hydrophilic linker is non-charged or negatively charged.

18. (canceled)

19. (canceled)

20. The method of claim 17, wherein the hydrophilic linker comprises a plurality of non-charged monomeric residues, at least one negatively charged residue, or a combination thereof.

21. (canceled)

22. (canceled)

23. (canceled)

24. (canceled)

25. The method of claim 20, wherein the hydrophilic linker has the following formula L1, L2, L3, or L4: 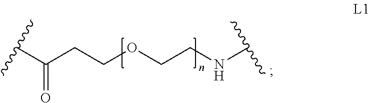 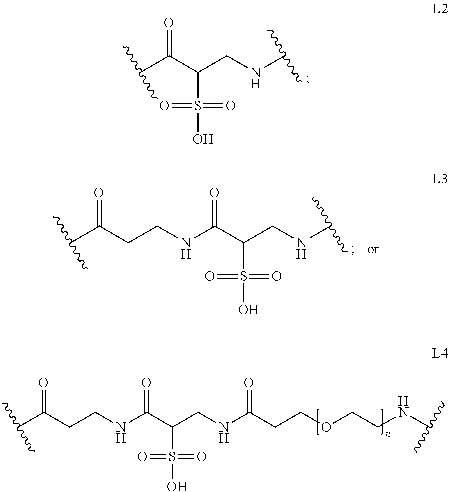
wherein n is an integer from four to 16 in the formulae L1 and L4, and [symbol] designates a point of attachment in the formulae L1, L2, L3, and L4.

26. (canceled)

27. The method of claim 20, wherein the at least one negatively charged residue comprises an anionic group selected from the group consisting of a sulfonate, a phosphate, a phosphonate, and a carboxylic acid.

28. (canceled)

29. (canceled)

30. (canceled)

31. (canceled)

32. (canceled)

33. (canceled)

34. (canceled)

35. The method of claim 12, wherein the cell-membrane-impermeable probe has the formula P1, P2, or P3 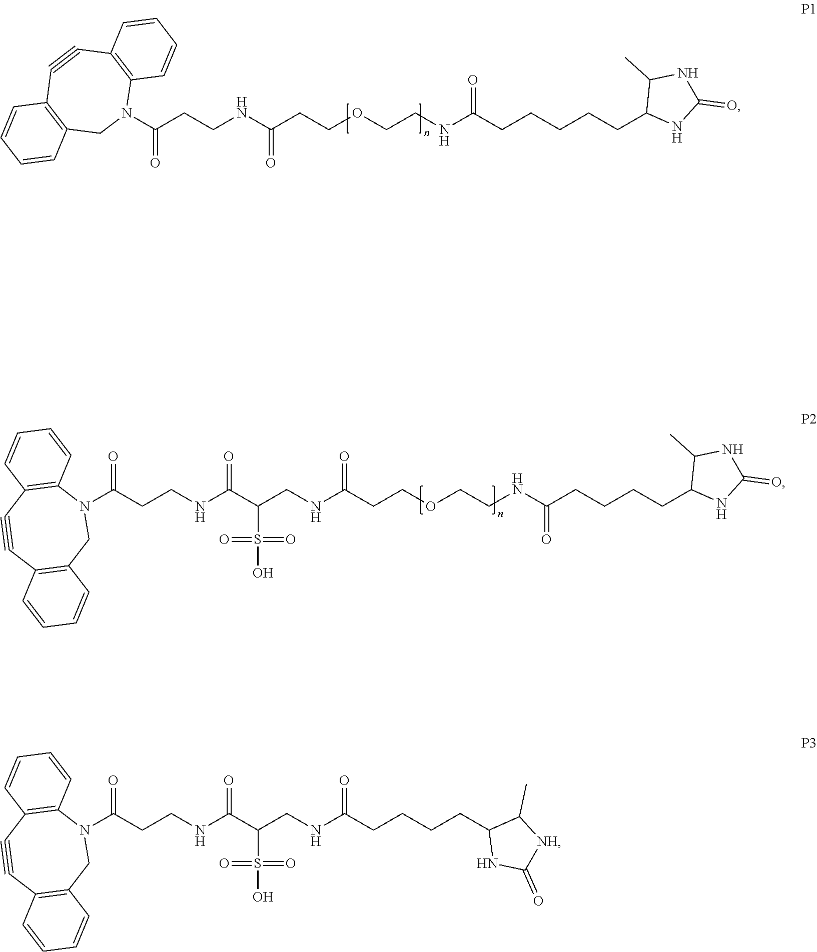
wherein n is an integer from 4 to 16.

36. (canceled)

37. (canceled)

38. The method of claim 1, wherein the cell culture system is a two-dimensional (2D) cell culture system or a three-dimensional (3D) cell culture system.

39. (canceled)

40. (canceled)

41. (canceled)

42. The method of claim 1, wherein the plurality of cells are from a cell line.

43. The method of claim 1, wherein the plurality of cells are from a primary tissue.

44. The method of claim 1, wherein the plurality of cells comprises tumor cells.

45. (canceled)

46. (canceled)

47. (canceled)

48. (canceled)

49. (canceled)

50. (canceled)

51. (canceled)

52. (canceled)

53. (canceled)

54. (canceled)

55. The method of claim 1, wherein the plurality of cells comprises a cell selected from the group consisting of fibroblasts, epithelial cells, endothelial cells, neuronal cells, glial cells, lymphocytes, macrophages, dendritic cells, hepatocytes, and stem cells.

56. The method of claim 1, wherein the plurality of cells comprises at least two different cell types.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. A method for isolating proteins that are pericellularly localized in a multicellular system, the method comprising:
(a) culturing a plurality of cells in a cell culture system, wherein the plurality of cells are cultured in the presence of an amino acid analog functionalized with a first chemical group that is reactive in a bioorthogonal chemical reaction, and wherein the amino acid analog is incorporated into newly synthesized proteins;
(b) contacting the cell culture system with a cell-membrane-impermeable probe having the formula A-L-D,
wherein
A is a molecule comprising a second chemical group that is reactive in a bioorthogonal chemical reaction, wherein the first chemical group is reactive with second chemical group,
L is a hydrophilic linker, and
D is a means for specifically binding to streptavidin with a dissociation constant greater than about 1 μM and an off-rate kinetic constant greater than about 1×10⁻³ l/s, and
wherein newly synthesized proteins that are pericellularly localized are labeled with the probe by a bioorthogonal chemical reaction between the first and second chemical groups;
(c) lysing the cultured cells to generate a lysate containing the labeled proteins and other cellular and extracellular components;
(d) contacting the lysate with a capture reagent comprising streptavidin immobilized on a solid support, wherein the streptavidin binds to the means for specifically binding to streptavidin on the labeled proteins to yield a [labeled protein]:probe complex; and
(e) separating the solid support from the lysate to separate the labeled proteins from the other cellular and extracellular components, thereby isolating the proteins that are pericellularly localized in the multicellular system.

147. (canceled)

148. (canceled)

149. (canceled)

150. (canceled)
